(19) United States
(12) Patent Application Publication
(10) Pub. No.: US 2009/0131436 A1
(43) Pub. Date: May 21, 2009

(54) Pyrimidine Derivatives

(76) Inventors: Patricia Imbach, Kaiseraugst (CH); Eiji Kawahara, Ibaraki (JP); Kazuhide Konishi, Ibaraki (JP); Naoko Matsuura, Ibaraki (JP); Takahiro Miyake, Ibaraki (JP); Osamu Ohmori, Ibaraki (JP); Johannes Roesel, Riehen (CH); Naoki Teno, Ibaraki (JP); Ichiro Umemura, Ibaraki (JP)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/574,019

(22) PCT Filed: Aug. 26, 2005
(86) PCT No.: PCT/EP2005/009251

§ 371 Date: Jan. 12, 2009

(30) Foreign Application Priority Data
Aug. 27, 2004 (GB) 0419161.5

(51) Int. Cl. A61K 31/5377; C07D 413/14; A61K 31/506; C07D 403/12; A61P 35/00

(52) U.S. Cl. 514/235.8; 544/121; 514/252.14; 544/295

(57) ABSTRACT

Novel pyrimidine derivatives of formula (I) to processes for their production, their use as pharmaceuticals and to pharmaceutical compositions comprising them.

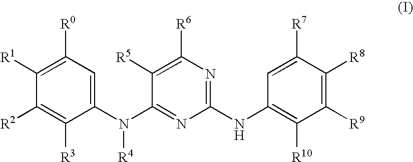

The present invention relates to novel pyrimidine derivatives, to processes for their production, their use as pharmaceuticals and to pharmaceutical compositions comprising them.

More particularly the present invention provides in a first aspect, a compound of formula I

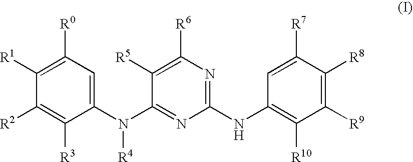

wherein
R₀ is hydrogen
R₁ is hydrogen or a 5 or 6 member heterocycl comprising 1 or 2 N atoms substituted by C₁-C₇alkyl, hydroxy, dialkylamino, or by a 6 member heterocycl comprising 1 N atom;
R₂ is hydrogen
R₃ is sulfonyl substituted once or twice by C₁-C₇alkyl; carbamoyl substituted once or twice by C₁-C₇alkyl; 5 or 6 member heterocycl comprising 1, 2, 3 or 4 N atoms; SO₂N(R₁₂)R₁₃ wherein R₁₂ is hydrogen or loweralkyl and R₁₃ is hydrogen, C₁-C₇alkyl, C₁-C₇alkoxy-C₁-C₇alkyl, di-C₁-C₇alkylamino-C₁-C₇alkyl, hydroxy-C₁-C₇alkyl or R₁₂ and R₁₃ together with the N to which they are attached form a heterocycl comprising 2 N atoms which is unsubstituted or substituted C₁-C₇alkyl;
R₂ and R₃ together with the N to which they are attached form a heterocycl comprising 2 hetero atoms independently selected from N or S which is unsubstituted or substituted once or twice by a substituent independently selected from loweralkyl and oxo;
R₄ is hydrogen
R₅ is halogen
R₆ is hydrogen
R₇ is hydrogen; C₁-C₇alkoxy; carbamoyl unsubstituted or substituted by loweralkyl; 5 or 6 member heterocycl comprising 1, 2 or 3 N or O atoms unsubstituted or substituted by di-C₁-C₇alkyl-amino, C₁-C₇alkyl, hydroxy, 5 or 6 member heterocycl comprising 1, 2 or 3 N or O atoms unsubstituted or substituted by C₁-C₇alkyl; 5 or 6 member heterocycloxy comprising 1, 2 or 3 N or O ring atoms unsubstituted or substituted by C₁-C₇alkyl; heterocycl-C₁-C₇alkyloxy wherein heterocycl is a 5 or 6 member heterocycl comprising 1, 2 or 3 N or O ring atoms unsubstituted or substituted by hydroxy or C₁-C₇alkyl;
R₈ is hydrogen; halogen; C1-C7alkoxy; carbamoyl unsubstituted or substituted by C₁-C₇alkyl; heterocycl-C₁-C₇alkyloxy wherein heterocycl is a 5 or 6 member heterocycl comprising 1, 2 or 3 N or O ring atoms unsubstituted or substituted by C₁-C₇alkyl, hydroxy; 5 or 6 member heterocycl comprising 1, 2 or 3 N or O atoms unsubstituted or substituted once or twice by a substituent independently selected from hydroxy, C₁-C₇alkoxy-C₁-C₇alkyl, C₁-C₇alkyl, aminocarbonyl and C₁-C₇alkylamino; 5 or 6 member heterocycloxy comprising 1 or 2 N ring atoms unsubstituted or substituted 1 to 5 times by C₁-C₇alkyl or di-C₁-C₇alkylamino; 10 member bi-cyclic-heterocycle comprising 1 to 3 heteroatoms selected from N or O;
R₇ and R₈ together with the atoms to which they are attached form a 6 member heterocycl comprising 1, 2 or 3 N or O atoms unsubstituted or substituted once or twice by C₁-C₇alkyl or oxo;
R₉ is hydrogen, 5 or 6 member heterocycl comprising 1, 2 or 3 N or O atoms unsubstituted or substituted by di-C₁-C₇alkyl-amino;
R₁₀ is hydrogen or C₁-C₇alkoxy, preferably C₁-C₇alkoxy;

Preferably a diphenyl-pyrimidine-diamine derivative selected from
2-{5-Bromo-2-[5-(3-dimethylamino-pyrrolidin-1-yl)-2-methoxy-phenylamino]-pyrimidin-4-ylamino}-N-methyl-benzenesulfonamide,
2-{5-Bromo-2-[2-methoxy-5-(2-morpholin-4-yl-ethoxy)-phenylamino]-pyrimidin-4-ylamino}-N-isopropyl-benzenesulfonamide,
7-[2-(4-[1,4′]Bipiperidinyl-1′-yl-2-methoxy-phenylamino)-5-chloro-pyrimidin-4-ylamino]-2-methyl-2,3-dihydro-isoindol-1-one,
2-[5-Chloro-2-(2-methoxy-4-morpholin-4-yl-phenylamino)-pyrimidin-4-ylamino]-5-(4-hydroxy-piperidin-1-yl)-N-methyl-benzamide,
5-[1,4′]Bipiperidinyl-1′-yl-2-[5-chloro-2-(2-methoxy-5-morpholin-4-yl-phenylamino)-pyrimidin-4-ylamino]-N-methyl-benzamide,
2-[2-(4-[1,4′]Bipiperidinyl-1′-yl-2-methoxy-phenylamino)-5-chloro-pyrimidin-4-ylamino]-N-isobutyl-benzenesulfonamide,
2-{5-Chloro-2-[2-methoxy-4-(4-methyl-piperazin-1-yl)-phenylamino]-pyrimidin-4-ylamino}-N-isobutyl-benzenesulfonamide,
2-[5-Chloro-2-(2-methoxy-5-morpholin-4-yl-phenylamino)-pyrimidin-4-ylamino]-5-(4-hydroxy-piperidin-1-yl)-N-methyl-benzamide,
2-[2-(5-[1,4′]Bipiperidinyl-1′-yl-2-methoxy-phenylamino)-5-bromo-pyrimidin-4-ylamino]-N-isopropyl-benzenesulfonamide,
1-{4-[5-Chloro-4-(2-isobutylsulfamoyl-phenylamino)-pyrimidin-2-ylamino]-3-methoxy-phenyl}-piperidine-4-carboxylic acid amide,
4-[5-Chloro-4-(2-isobutylsulfamoyl-phenylamino)-pyrimidin-2-ylamino]-3-methoxy-N-methyl-benzamide,
2-{5-Chloro-2-[4-(4-hydroxy-piperidin-1-yl)-2-methoxy-phenylamino]-pyrimidin-4-ylamino}-N-isobutyl-benzenesulfonamide,
3-[5-Chloro-4-(2-isobutylsulfamoyl-phenylamino)-pyrimidin-2-ylamino]-4-methoxy-N-methyl-benzamide,
5-Chloro-N²-(2-methoxy-4-yl-phenyl)-N⁴-[2-(2H-tetrazol-5-yl)-phenyl]-pyrimidine-2,4-diamine,
2-{5-Chloro-2-[4-((S)-3-dimethylamino-pyrrolidin-1-yl)-2-methoxy-phenylamino]-pyrimidin-4-ylamino}-N-isobutyl-benzenesulfonamide,
2-{5-Chloro-2-[2-methoxy-4-(1-methyl-piperidin-4-yloxy)-phenylamino]-pyrimidin-4-ylamino}-N-isobutyl-benzenesulfonamide,
7-{5-Chloro-2-[4-((S)-3-dimethylamino-pyrrolidin-1-yl)-2-methoxy-phenylamino]-pyrimidin-4-ylamino}-2-methyl-2,3-dihydro-isoindol-1-one,
2-{5-Chloro-2-[4-((S)-3-dimethylamino-pyrrolidin-1-yl)-2-methoxy-phenylamino]-pyrimidin-4-ylamino}-N-methyl-5-(4-methyl-piperazin-1-yl)-benzamide,
1-{4-[5-Chloro-4-(2-methylcarbamoyl-phenylamino)-pyrimidin-2-ylamino]-3-methoxy-phenyl}-3-methyl-piperidine-3-carboxylic acid amide,
1-[4-[5-Chloro-4-(2-methyl-3-oxo-2,3-dihydro-1H-isoindol-4-ylamino)-pyrimidin-2-ylamino]-3-methoxy-phenyl)-3-methyl-piperidine-3-carboxylic acid amide,
1-{4-[5-Chloro-4-(2-isobutylsulfamoyl-phenylamino)-pyrimidin-2-ylamino]-3-methoxy-phenyl}-3-methyl-piperidine-3-carboxylic acid amide,
2-{5-Chloro-2-[5-(3-dimethylamino-pyrrolidin-1-yl)-2-methoxy-phenylamino]-pyrimidin-4-ylamino}-N-methyl-5-(4-methyl-piperazin-1-yl)-benzamide,
7-{5-Chloro-2-[2-methoxy-4-(1-methyl-piperidin-4-yloxy)-phenylamino]-pyrimidin-4-ylamino}-2-methyl-2,3-dihydro-isoindol-1-one,
2-[5-Bromo-2-(2,5-dimethoxy-4-morpholin-4-yl-phenylamino)-pyrimidin-4-ylamino]-N-methyl-benzenesulfonamide,
2-{5-Bromo-2-[5-(4-hydroxy-piperidin-1-yl)-2-methoxy-phenylamino]-pyrimidin-4-ylamino}-N-methyl-benzenesulfonamide,
2-[5-Chloro-2-(2-methoxy-5-morpholin-4-yl-phenylamino)-pyrimidin-4-ylamino]-N-isobutyl-benzenesulfonamide,
2-{5-Chloro-2-[2-methoxy-5-(4-methyl-piperazin-1-yl)-phenylamino]-pyrimidin-4-ylamino}-N-isobutyl-benzenesulfonamide,
2-[2-(5-[1,4′]Bipiperidinyl-1′-yl-2-methoxy-phenylamino)-5-chloro-pyrimidin-4-ylamino]-N-isobutyl-benzenesulfonamide,
2-{5-Chloro-2-[5-((S)-3-dimethylamino-pyrrolidin-1-yl)-2-methoxy-phenylamino]-pyrimidin-4-ylamino}-N-isobutyl-benzenesulfonamide,
1-{4-[5-Chloro-4-(2-methyl-3-oxo-2,3-dihydro-1H-isoindol-4-ylamino)-pyrimidin-2-ylamino]-3-methoxy-phenyl}-piperidine-4-carboxylic acid amide,
2-[5-Chloro-2-(2-methoxy-4-morpholin-4-yl-phenylamino)-pyrimidin-4-ylamino]-5-((S)-3-dimethylamino-pyrrolidin-1-yl)-N-methyl-benzamide,
7-{5-Chloro-2-[4-(4-isopropyl-piperazin-1-yl)-2-methoxy-phenylamino]-pyrimidin-4-ylamino}-2-methyl-2,3-dihydro-isoindol-1-one,
2-[5-Chloro-2-(2-methoxy-4-morpholin-4-yl-phenylamino)-pyrimidin-4-ylamino]-N-(2,2-dimethyl-propyl)-benzenesulfonamide,
2-{5-Chloro-2-[2-methoxy-4-(4-methyl-piperazin-1-yl)-phenylamino]-pyrimidin-4-ylamino}-N-(2,2-dimethyl-propyl)-benzenesulfonamide,
3-[5-Chloro-4-(2-isobutylsulfamoyl-phenylamino)-pyrimidin-2-ylamino]-4-methoxy-benzamide,
2-[5-Bromo-2-(2,4-dimethoxy-5-morpholin-4-yl-phenylamino)-pyrimidin-4-ylamino]-N-methyl-benzenesulfonamide,
2-{5-Bromo-2-[5-(1-isopropyl-piperidin-4-yloxy)-2-methoxy-phenylamino]-pyrimidin-4-ylamino}-N-methyl-benzenesulfonamide,
7-(5-Chloro-2-{2-methoxy-4-[2-(4-methyl-piperazin-1-yl)-ethoxy]-phenylamino}-pyrimidin-4-ylamino)-2-methyl-2,3-dihydro-isoindol-1-one,
2-(5-Chloro-2-{2-methoxy-4-[4-(4-methyl-piperazin-1-yl)-piperidin-1-yl]-phenylamino}-pyrimidin-4-ylamino)-N-isobutyl-benzenesulfonamide,
(S)-1-{4-[5-Chloro-4-(2-methyl-3-oxo-2,3-dihydro-1H-isoindol-4-ylamino)-pyrimidin-2-ylamino]-3-methoxy-phenyl}-3-methyl-piperidine-3-carboxylic acid amide,
(S)-1-{4-[5-Chloro-4-(2-methylcarbamoyl-phenylamino)-pyrimidin-2-ylamino]-3-methoxy-phenyl}-3-methyl-piperidine-3-carboxylic acid amide,
7-[5-Chloro-2-(2,4-dimethoxy-phenylamino)-pyrimidin-4-ylamino]-2-methyl-2,3-dihydro-isoindol-1-one,
2-(5-Bromo-2-{2-methoxy-5-[4-(4-methyl-piperazin-1-yl)-piperidin-1-yl]-phenylamino}-pyrimidin-4-ylamino)-N-methyl-benzenesulfonamide,
2-{5-Bromo-2-[5-(4-hydroxy-piperidin-1-yl)-2-methoxy-phenylamino]-pyrimidin-4-ylamino}-N-isopropyl-benzenesulfonamide,
2-{5-Bromo-2-[2-methoxy-5-(1-methyl-piperidin-4-yloxy)-phenylamino]-pyrimidin-4-ylamino}-N-isopropyl-benzenesulfonamide,
2-{5-Bromo-2-[5-(1-isopropyl-piperidin-4-yloxy)-2-methoxy-phenylamino]-pyrimidin-4-ylamino}-N-isopropyl-benzenesulfonamide,
7-{5-Chloro-2-[2-methoxy-4-(2-morpholin-4-yl-ethoxy)-phenylamino]-pyrimidin-4-ylamino}-2-methyl-2,3-dihydro-isoindol-1-one,
7-{5-Chloro-2-[2-methoxy-5-(2-morpholin-4-yl-ethoxy)-phenylamino]-pyrimidin-4-ylamino}-2-methyl-2,3-dihydro-isoindol-1-one,
7-{5-Chloro-2-[4-(1-isopropyl-piperidin-4-yloxy)-2-methoxy-phenylamino]-pyrimidin-4-ylamino}-2-methyl-2,3-dihydro-isoindol-1-one,
2-{5-Bromo-2-[5-(3-dimethylamino-pyrrolidin-1-yl)-2-methoxy-phenylamino]-pyrimidin-4-ylamino}-N-isopropyl-benzenesulfonamide,
2-{5-Bromo-2-[2-methoxy-5-(4-methyl-piperazin-1-yl)-phenylamino]-pyrimidin-4-ylamino}-N-isopropyl-benzenesulfonamide,
2-(5-Bromo-2-{2-methoxy-5-[4-(4-methyl-piperazin-1-yl)-piperidin-1-yl]-phenylamino}-pyrimidin-4-ylamino)-N-isopropyl-benzenesulfonamide,
7-{5-Chloro-2-[2-methoxy-4-(1,2,2,6,6-pentamethyl-piperidin-4-yloxy)-phenylamino]-pyrimidin-4-ylamino}-2-methyl-2,3-dihydro-isoindol-1-one,
1-{4-[5-Chloro-4-(2-methyl-3-oxo-2,3-dihydro-1H-isoindol-4-ylamino)-pyrimidin-2-ylamino]-3-methoxy-phenyl}-piperidine-3-carboxylic acid amide,
2-{5-Chloro-2-[2-methoxy-4-(1,2,2,6,6-pentamethyl-piperidin-4-yloxy)phenylamino]-pyrimidin-4-ylamino}-N-isobutyl-benzenesulfonamide,
(R)-1-{4-[5-Chloro-4-(2-methylcarbamoyl-phenylamino)-pyrimidin-2-ylamino]-3-methoxy-phenyl}-3-methyl-piperidine-3-carboxylic acid amide,
(R)-1-{4-[5-Chloro-4-(2-methyl-3-oxo-2,3-dihydro-1H-isoindol-4-ylamino)-pyrimidin-2-ylamino]-3-methoxy-phenyl}-3-methyl-piperidine-3-carboxylic acid amide,
2-{5-Chloro-2-[2-methoxy-4-((R)-1-methyl-pyrrolidin-2-ylmethoxy)-phenylamino]-pyrimidin-4-ylamino}-N-isobutyl-benzenesulfonamide,
2-{5-Chloro-2-[2-methoxy-4-((S)-1-methyl-pyrrolidin-2-ylmethoxy)-phenylamino]-pyrimidin-4-ylamino}-N-isobutyl-benzenesulfonamide,
2-{5-Bromo-2-[2-methoxy-5-(2-piperidin-1-yl-ethoxy)-phenylamino]-pyrimidin-4-ylamino}-N-methyl-benzenesulfonamide,
2-(5-Bromo-2-{5-[2-(4-hydroxy-piperidin-1-yl)-ethoxy]-2-methoxy-phenylamino}-pyrimidin-4-ylamino)-N-methyl-benzenesulfonamide,
5-Chloro-N⁴-(1,1-dioxo-1λ⁶-thiochroman-8-yl)-N²-(2-methoxy-4-morpholin-4-yl-phenyl)-pyrimidine-2,4-diamine,
2-[5-Chloro-2-(2-methoxy-4-morpholin-4-yl-phenylamino)-pyrimidin-4-ylamino]-N-(2-hydroxy-ethyl)-benzenesulfonamide,
2-[5-Chloro-2-(2-methoxy-4-morpholin-4-yl-phenylamino)-pyrimidin-4-ylamino]-N-(2-methoxy-ethyl)-benzenesulfonamide,
7-{5-Chloro-2-[2-methoxy-4-(2-piperidin-1-yl-ethoxy)-phenylamino]-pyrimidin-4-ylamino}-2-methyl-2,3-dihydro-isoindol-1-one,
2-[5-Chloro-2-(2-methoxy-4-morpholin-4-yl-phenylamino)-pyrimidin-4-ylamino]-N-((R)-2-hydroxy-propyl)-benzenesulfonamide,
2-[5-Chloro-2-(2-methoxy-4-morpholin-4-yl-phenylamino)-pyrimidin-4-ylamino]-N-(3-hydroxy-propyl)-benzenesulfonamide,
2-[5-Chloro-2-(2-methoxy-4-morpholin-4-yl-phenylamino)-pyrimidin-4-ylamino]-N-((S)-2-hydroxy-propyl)-benzenesulfonamide,
2-{5-Bromo-2-[2-methoxy-5-(4-morpholin-4-yl-piperidin-1-yl)-phenylamino]-pyrimidin-4-ylamino}-N-isopropyl-benzenesulfonamide,
7-(5-Chloro-2-{2-methoxy-4-[(S)-4-(2-methoxy-ethyl)-3-methyl-piperazin-1-yl]-phenylamino}-pyrimidin-4-ylamino)-2-methyl-2,3-dihydro-isoindol-1-one,
7-(5-Chloro-2-{2-methoxy-4-[(R)-4-(2-methoxy-ethyl)-3-methyl-piperazin-1-yl]-phenylamino}-pyrimidin-4-ylamino)-2-methyl-2,3-dihydro-isoindol-1-one,
5-Chloro-N²-[4-((S)-3-dimethylamino-pyrrolidin-1-yl)-2-methoxy-phenyl]-N⁴-(1,1-dioxo-1λ⁶-thiochroman-8-yl)-pyrimidine-2,4-diamine,
5-Chloro-N⁴-(1,1-dioxo-1λ⁶-thiochroman-8-yl)-N²-{2-methoxy-4-[4-(4-methyl-piperazin-1-yl)-piperidin-1-yl]-phenyl}-pyrimidine-2,4-diamine,
2-{5-Bromo-2-[2-methoxy-5-(4-morpholin-4-yl-piperidin-1-yl)-phenylamino]-pyrimidin-4-ylamino}-N-methyl-benzenesulfonamide,
2-[5-Bromo-2-(4-fluoro-2-methoxy-5-morpholin-4-yl-phenylamino)-pyrimidin-4-ylamino]-N-methyl-benzenesulfonamide,
4-[5-Chloro-4-(1,1-dioxo-1λ⁶-thiochroman-8-ylamino)-pyrimidin-2-ylamino]-3-methoxy-N-methyl-benzamide,
2-(5-Bromo-2-[2-methoxy-5-((S)-1-methyl-pyrrolidin-2-ylmethoxy)-phenylamino]-pyrimidin-4-ylamino)-N-methyl-benzenesulfonamide,
2-{5-Bromo-2-[2-methoxy-5-((R)-1-methyl-pyrrolidin-2-ylmethoxy)phenylamino]-pyrimidin-4-ylamino}-N-methyl-benzenesulfonamide,
2-{5-Bromo-2-[2,4-dimethoxy-5-(2-morpholin-4-yl-ethoxy)-phenylamino]-pyrimidin-4-ylamino}-N-methyl-benzenesulfonamide,
2-[5-Chloro-2-(2-methoxy-4-morpholin-4-yl-phenylamino)-pyrimidin-4-ylamino]-N-isopropyl-N-methyl-benzenesulfonamide,
2-[5-Chloro-2-(2-methoxy-4-morpholin-4-yl-phenylamino)-pyrimidin-4-ylamino]-N-methyl-N-propyl-benzenesulfonamide,
7-(5-Chloro-2-{4-[2-(4-isopropyl-piperazin-1-yl)-ethoxy]-2-methoxy-phenylamino}-pyrimidin-4-ylamino)-2-methyl-2,3-dihydro-isoindol-1-one,
2-{5-Bromo-2-[2-methoxy-5-(2-morpholin-4-yl-ethoxy)-phenylamino]-pyrimidin-4-ylamino}-N,N-dimethyl-benzenesulfonamide,
2-[5-Bromo-2-(2,4-dim ethoxy-5-morpholin-4-yl-phenylamino)-pyrimidin-4-ylamino]-N-isopropyl-benzenesulfonamide,
2-[5-Chloro-2-(2-methoxy-4-morpholin-4-yl-phenylamino)-pyrimidin-4-ylamino]-N-(2-dimethylaminoethyl)-benzenesulfonamide,
5-Chloro-N²-(2-methoxy-4-morpholin-4-yl-phenyl)-N⁴-[2-(4-methyl-piperazine-1-sulfonyl)-phenyl]-pyrimidine-2,4-diamine,
2-[5-Chloro-2-(2-methoxy-4-morpholin-4-yl-phenylamino)-pyrimidin-4-ylamino]-N-(2-ethoxy-ethyl)-benzenesulfonamide,
2-[5-Bromo-2-(7-methoxy-4-methyl-3-oxo-3,4-dihydro-2H-benzo[1,4]oxazin-6-ylamino)-pyrimidin-4-ylamino]-N-methyl-benzenesulfonamide,
2-[5-Bromo-2-(2-methoxy-5-morpholin-4-yl-phenylamino)-pyrimidin-4-ylamino]-N,N dimethyl-benzenesulfonamide,
2-[5-Bromo-2-(2-methoxy-5-morpholin-4-yl-phenylamino)-pyrimidin-4-ylamino]-N,N-dimethyl-benzenesulfonamide,
2-{5-Chloro-2-[2-methoxy-4-(4-methyl-piperazin-1-yl)-phenylamino]-pyrimidin-4-ylamino}-N-methyl-N-propyl-benzenesulfonamide,
2-[5-Bromo-2-(2-methoxy-5-piperidin-1-yl-phenylamino)-pyrimidin-4-ylamino]-N-methyl-benzenesulfonamide,
2-[5-Bromo-2-(2-methoxy-5-piperidin-1-yl-phenylamino)-pyrimidin-4-ylamino]-N-isopropyl-benzenesulfonamide,
7-{5-Chloro-2-[4-((R)-3-dimethylamino-pyrrolidin-1-yl)-2-methoxy-phenylamino]-pyrimidin-4-ylamino}-2-methyl-2,3-dihydro-isoindol-1-one,
5-Chloro-N²-(2-methoxy-4-morpholin-4-yl-phenyl)-N⁴-[2-(piperazine-1-sulfonyl)-phenyl]-pyrimidine-2,4-diamine,
2-[5-Chloro-2-(2-methoxy-4-morpholin-4-yl-phenylamino)-pyrimidin-4-ylamino]-N-isobutyl-N-methyl-benzenesulfonamide,
2-[5-Chloro-2-(2-methoxy-4-morpholin-4-yl-phenylamino)-pyrimidin-4-ylamino]-N-ethyl-N-methyl-benzenesulfonamide,
2-{5-Chloro-2-[2-methoxy-4-(4-methyl-piperazin-1-yl)-phenylamino]-pyrimidin-4-ylamino}-N-isobutyl-N-methyl-benzenesulfonamide,
2-{5-Chloro-2-[2-methoxy-4-(4-methyl-piperazin-1-yl)-phenylamino]-pyrimidin-4-ylamino}-N-ethyl-N-methyl-benzenesulfonamide,
7-(5-Chloro-2-{4-[2-(4-hydroxy-piperidin-1-yl)-ethoxy]-2-methoxy-phenylamino}pyrimidin-4-ylamino)-2-methyl-2,3-dihydro-isoindol-1-one,
2-(5-Bromo-2-{2-methoxy-5-[4-(4-methyl-piperazin-1-yl)-piperidin-1-yl]-phenylamino}-pyrimidin-4-ylamino)-N,N-dimethyl-benzenesulfonamide,
8-{5-Chloro-2-[2-methoxy-4-(4-methyl-piperazin-1-yl)-phenylamino]-pyrimidin-4-ylamino}-2-methyl-3,4-dihydro-2H-isoquinolin-1-one,
8-[5-Chloro-2-(2-methoxy-4-morpholin-4-yl-phenylamino)-pyrimidin-4-ylamino]-2-methyl-3,4-dihydro-2H-isoquinolin-1-one,
8-(5-Chloro-2-{2-methoxy-4-[4-(4-methyl-piperazin-1-yl)-piperidin-1-yl]-phenylamino}-pyrimidin-4-ylamino)-2-methyl-3,4-dihydro-2H-isoquinolin-1-one,
8-[2-(4-[1,4′]Bipiperidinyl-1′-yl-2-methoxy-phenylamino)-5-chloro-pyrimidin-4-ylamino]-2-methyl-3,4-dihydro-2H-isoquinolin-1-one,
8-{5-Chloro-2-[4-(4-hydroxy-piperidin-1-yl)-2-methoxy-phenylamino]-pyrimidin-4-ylamino}-2-methyl-3,4-dihydro-2H-isoquinolin-1-one,
8-{5-Chloro-2-[4-(4-isopropyl-piperazin-1-yl)-2-methoxy-phenylamino]-pyrimidin-4-ylamino}-2-methyl-3,4-dihydro-2H-isoquinolin-1-one,
7-(5-Chloro-2-(2-methoxy-4-[3-(4-methyl-piperazin-1-yl)-propoxy]-phenylamino)pyrimidin-4-ylamino)-2-methyl-2,3-dihydro-isoindol-1-one,
8-{5-Chloro-2-[4-((S)-3-dimethylamino-pyrrolidin-1-yl)-2-methoxy-phenylamino]-pyrimidin-4-ylamino}-2-methyl-3,4-dihydro-2H-isoquinolin-1-one,
8-{5-Chloro-2-[4-((R)-3-dimethylamino-pyrrolidin-1-yl)-2-methoxy-phenylamino]-pyrimidin-4-ylamino}-2-methyl-3,4-dihydro-2H-isoquinolin-1-one,
8-[5-Chloro-2-((S)-4-hexahydro-pyrazino[2,1-c][1,4]oxazin-8-yl-2-methoxy-phenylamino)-pyrimidin-4-ylamino]-2-methyl-3,4-dihydro-2H-isoquinolin-1-one,
8-[5-Chloro-2-((R)-4-hexahydro-pyrazino[2,1-c][1,4]oxazin-8-yl-2-methoxy-phenylamino)-pyrimidin-4-ylamino]-2-methyl-3,4-dihydro-2H-isoquinolin-1-one,
8-[5-Chloro-2-(2-methoxy-4-morpholin-4-yl-phenylamino)-pyrimidin-4-ylamino]-2-ethyl-3,4-dihydro-2H-isoquinolin-1-one,
8-(5-Chloro-2-{2-methoxy-4-[4-(4-methyl-piperazin-1-yl)-piperidin-1-yl]-phenylamino}-pyrimidin-4-ylamino)-2-ethyl-3,4-dihydro-2H-isoquinolin-1-one,
2-[5-Chloro-2-(2-methoxy-5-morpholin-4-yl-phenylamino)-pyrimidin-4-ylamino]-N-methyl-5-(4-methyl-piperazin-1-yl)-benzamide,
5-[1,4′]Bipiperidinyl-1′-yl-2-[5-chloro-2-(2-methoxy-4-morpholin-4-yl-phenylamino)-pyrimidin-4-ylamino]-N-methyl-benzamide,
2-[5-Bromo-2-(2-methoxy-5-morpholin-4-yl-phenylamino)-pyrimidin-1-ylamino]-N-isopropyl-benzenesulfonamide,
5-Chloro-N²-{4-[4-(4-ethyl-piperazin-1-yl)-piperidin-1-yl]-2-methoxy-phenyl}-N⁴-[2-(propane-2-sulfonyl)-phenyl]-pyrimidine-2,4-diamine,
2-{5-Chloro-2-[4-((S)-3-ethylamino-pyrrolidin-1-yl)-2-methoxy-phenylamino]-pyrimidin-4-ylamino}-N-isopropyl-benzenesulfonamide,
2-{5-Chloro-2-[4-((R)-3-ethylamino-pyrrolidin-1-yl)-2-methoxy-phenylamino]-pyrimidin-4-ylamino}-N-isopropyl-benzenesulfonamide,
2-{5-Chloro-2-[2-methoxy-4-((S)-3-methylamino-pyrrolidin-1-yl)-phenylamino]-pyrimidin-4-ylamino}-N-isopropyl-benzenesulfonamide,
2-{5-Chloro-2-[2-methoxy-4-((R)-3-methylamino-pyrrolidin-1-yl)-phenylamino]-pyrimidin-4-ylamino}-N-isopropyl-benzenesulfonamide,
2-{5-Chloro-2-[4-((R)-3-dimethylamino-pyrrolidin-1-yl)-2-methoxy-phenylamino]-pyrimidin-4-ylamino}-N-isopropyl-benzenesulfonamide,
2-{5-Chloro-2-[4-((S)-3-dimethylamino-pyrrolidin-1-yl)-2-methoxy-phenylamino]-pyrimidin-4-ylamino}-N-isopropyl-benzenesulfonamide,
2-{5-Chloro-2-[2-ethoxy-4-(4-methyl-piperazin-1-yl)-phenylamino]-pyrimidin-4-ylamino}-N-isopropyl-benzenesulfonamide,
2-{5-Chloro-2-[2-isopropoxy-4-(4-methyl-piperazin-1-yl)-phenylamino]-pyrimidin-4-ylamino}-N-isopropyl-benzenesulfonamide,
2-{5-Chloro-2-[2-cyclopropylmethoxy-4-(4-methyl-piperazin-1-yl)-phenylamino]-pyrimidin-4-ylamino}-N-isopropyl-benzenesulfonamide
   and salts thereof.

The general terms used hereinbefore and hereinafter preferably have within the context of this disclosure the following meanings, unless otherwise indicated:

Where the plural form is used for compounds, salts, and the like, this is taken to mean also a single compound, salt, or the like.

Any asymmetric carbon atoms may be present in the (R)-, (S)- or (R,S)-configuration, preferably In the (R)- or (S)-configuration. The compounds may thus be present as mixtures of isomers or as pure isomers, preferably as enantiomer-pure diastereomers.

The invention relates also to possible tautomers of the compounds of formula I.

C₁-C₈alkyl denotes a an alkyl radical having from 1 up to 8, especially up to 4 carbon atoms, the radicals in question being either linear or branched with single or multiple branching; preferably, C₁-C₈alkyl is butyl, such as n-butyl, sec-butyl, isobutyl, tert-butyl, propyl, such as n-propyl or isopropyl, ethyl or methyl; especially methyl, propyl or tert-butyl.

C₂-C₈alkenyl denotes a an alkenyl radical having from 2 up to 8, especially up to 5 carbon atoms, the radicals in question being either linear or branched with single or multiple branching; preferably, C₂-C₈alkenyl is pentenyl, such as 3-methyl-2-buten-2-yl, butenyl, such as 1 or 2-butenyl or 2-buten-2-yl, propenyl, such as 1-propenyl or allyl, or vinyl.

C₂-C₈alkinyl denotes a an alkinyl radical having from 2 up to 8, especially up to 5 carbon atoms, the radicals in question being either linear or branched; preferably, C₂-C₈alkinyl is propinyl, such as 1-propinyl or propargyl, or acetylenyl.

C₃-C₈cycloalkyl denotes a cycloalkyl radical having from 3 up to 8 carbon atoms, such as cyclopropyl, cyclobutyl, cyclopentyl, cyclohexyl, cycloheptyl or cyclooctyl, preferably cyclopropyl, cyclopentyl or cyclohexyl.

C₁-C₈alkoxy is especially methoxy, ethoxy, isopropyloxy, or tert-butoxy.

HydroxyC₁-C₈alkyl is especially hydroxymethyl, 2-hydroxyethyl or 2-hydroxy-2-propyl.

HydroxyC₁-C₈alkoxy is especially 2-hydroxyethoxy or 3-hydroxypropoxy.

C₁-C₈alkoxyC₁-C₈alkoxy is especially 2-methoxyethoxy.

C₁-C₈alkoxyC₁-C₈alkyl is especially methoxymethyl, 2-methoxyethyl or 2-ethoxyethyl.

Halogen is preferably fluorine, chlorine, bromine, or iodine, especially fluorine, chlorine, or bromine.

HaloC₁-C₈alkyl is preferably chloroC₁-C₈alkyl or fluoroC₁-C₈alkyl, especially trifluoromethyl or pentafluoroethyl.

HaloC₁-C₈alkoxy is preferably chloroC₁-C₈alkoxy or fluoroC₁-C₈alkoxy, especially trifluoromethoxy.

C₁-C₈alkoxycarbonyl is especially tert-butoxycarbonyl, iso-propoxycarbonyl, methoxycarbonyl or ethoxycarbonyl.

Unsubstitued or substituted carbamoyl is carbamoyl substituted by one or two substituents selected from hydrogen, C₁-C₈alkyl, C₂-C₈alkenyl, C₂-C₈alkinyl, C₃-C₈cycloalkyl, C₃-C₈cycloalkylC₁-C₈alkyl, C₅-C₁₀arylC₁-C₈alkyl, hydroxyC₁-C₈alkyl, C₁-C₈alkoxyC₁-C₈alkyl, haloC₁-C₈alkyl, unsubstituted or substituted C₅-C₁₀aryl, or aminoC₁-C₈alkyl, or carbamoyl wherein the substituents and the nitrogen atom of the carbamoyl group represent a 5 or 6 membered heterocyclyl further comprising 0, 1 or 2 hetero atoms selected from N, O and S; and is preferably carbamoyl, methylcarbamoyl, dimethylcarbamoyl, propylcarbamoyl, hydroxyethyl-methyl-carbamoyl, di(hydroxyethyl)carbamoyl, dimethylaminoethylcarbamoyl, or pyrrolidinocarbonyl, piperidinocarbonyl, N-methylpiperazinocarbonyl or morpholinocarbonyl, especially carbamoyl or dimethylcarbamoyl.

Unsubstitued or substituted sulfamoyl is sulfamoyl substituted by one or two substituents selected from hydrogen, C₁-C₈alkyl, C₂-C₈alkenyl, C2-C₈alkinyl, C₃-C₈cycloalkyl, C₃-C₈cycloalkylC₁-C₈alkyl, C₅-C₁₀arylC₁-C₈alkyl, hydroxyC₁-C₈alkyl, C₁-C₈alkoxyC₁-C₈alkyl, haloC₁-C₈alkyl, unsubstituted or substituted C₅-C₁₀aryl, or aminoC₁-C₈alkyl, or sulfamoyl wherein the substituents and the nitrogen atom of the sulfamoyl group represent a 5 or 6 membered heterocyclyl further comprising 0, 1 or 2 hetero atoms selected from N, O and S; and is preferably sulfamoyl, methylsulfamoyl, propylsulfamoyl, cyclopropylmethyl-sulfamoyl, 2,2,2-trifluoroethylsulfamoyl, dimethylaminoethylsulfamoyl, dimethylsulfamoyl, hydroxyethyl-methyl-sulfamoyl, di(hydroxyethyl)sulfamoyl, or pyrrolidinosulfonyl, piperidinosulfonyl, N-methylpiperazinosulfonyl or morpholinosulfonyl, especially sulfamoyl or methylsulfamoyl.

Unsubstitued or substituted amino is amino substituted by one or two substituents selected from hydrogen, C₁-C₈alkyl, C₂-C₈alkenyl, C₂-C₈alkinyl, C₃-C₈cycloalkyl, C₃-C₈cycloalkylC₁-C₈alkyl, C₅-C₁₀arylC₁-C₈alkyl, hydroxyC₁-C₈alkyl, C₁-C₈alkoxyC₁-C₈alkyl, haloC₁-C₈alkyl, unsubstituted or substituted C₅-C₁₀aryl, aminoC₁-C₈alkyl, acyl, e.g. formyl, C₁-C₈alkylcarbonyl, C₅-C₁₀arylcarbonyl, C₁-C₈alkylsulfonyl or C₅-C₁₀arylsulfonyl, and is preferably amino, methylamino, dimethylamino, propylamino, benzylamino, hydroxyethyl-methyl-amino, di(hydroxyethyl)amino, dimethylaminoethylamino, acetylamino, acetyl-methyl-amino, benzoylamino, methylsulfonylamino or phenylsulfonylamino, especially amino or dimethylamino.

AminoC₁-C₈alkyl is especially aminoethyl, methylaminoethyl, dimethylaminoethyl or dimethylaminopropyl.

Unsubstitued or substituted C₅-C₁₀aryl is, for example, phenyl, indenyl, indanyl, naphthyl, or 1,2,3,4-tetrahydronaphthalenyl, optionally substituted by C₁-C₈alkyl, C₁-C₈alkoxyC₁-C₈alkyl, haloC₁-C₈alkyl, hydroxy, C₁-C₈alkoxy, methylenedioxy, amino, substituted amino, halogen, carboxy, C₁-C₈alkoxycarbonyl, carbamoyl, sulfamoyl, cyano or nitro; preferably phenyl, tolyl, trifluoromethylphenyl, methoxyphenyl, dimethoxyphenyl, methylenedioxyphenyl, chlorophenyl or bromophenyl, whereby the substituents may be in ortho, meta or para position, preferably meta or para.

C₅-C₁₀aryloxy is especially phenoxy or methoxyphenoxy, e.g. p-methoxyphenoxy.

C₅-C₁₀arylC₁-C₈alkyl is especially benzyl or 2-phenylethyl.

C₅-C₁₀arylC₁-C₈alkoxy is especially benzyloxy or 2-phenylethoxy.

Unsubstitued or substituted 5 or 6 membered heterocyclyl comprising 1, 2 or 3 hetero atoms selected from N, O and S may be unsaturated, partially unsaturated or saturated, and further condensed to a benzo group or a 5 or 6 membered heterocyclyl group, and may be bound through a hetero or a carbon atom, and is, for example, pyrrolyl, indolyl, pyrrolidinyl, imidazolyl, benzimidazolyl, pyrazolyl, triazolyl, benzotriazolyl, tetrazolyl, pyridyl, quinolinyl, isoquinolinyl, 1,2,3,4-tetrahydroquinolinyl, piperidyl, pyrimidinyl, pyrazinyl, piperazinyl, purinyl, tetrazinyl, oxazolyl, isoxalyl, morpholinyl, thiazolyl, benzothiazolyl, oxadiazolyl, and benzoxadiazolyl. Substituents considered are C₁-C₈alkyl, hydroxyC₁-C₈alkyl, C₁-C₈alkoxyC₁-C₈alkyl, C₁-C₈alkoxyC₁-C₈alkoxy, haloC₁-C₈alkyl, hydroxy, amino, substituted amino, C₁-C₈alkoxy, halogen, carboxy, C₁-C₈alkylcarbonyl, C₁-C₈alkoxycarbonyl, carbamoyl, C₁-C₈alkylcarbamoyl, cyano, oxo, or unsubstituted or substituted 5 or 6 membered heterocyclyl as defined in this paragraph. 5 or 6 membered heterocyclyl preferably comprises 1 or 2 hetero atoms selected from N, O and S, and is especially indolyl, pyrrolidinyl, pyrrolidonyl, imidazolyl, N-methylimidazolyl, benzimidazolyl, S,S-dioxoisothiazolidinyl, piperidyl, 4-acetylaminopiperidyl, 4-methylcarbamoylpiperidyl, 4-piperidinopiperidyl, 4-cyanopiperidyl, piperazinyl, N-methylpiperazinyl, N-(2-hydroxyethyl)piperazinyl, morpholinyl, 1-aza-2,2-dioxo-2-thiacyclohexyl, or sulfolanyl.

In unsubstituted or substituted heterocyclyloxy, heterocyclyl has the meaning as defined above, and is especially N-methyl-4-piperidyloxy. In unsubstituted or substituted heterocyclylC₁-C₈alkoxy, heterocyclyl has the meaning as defined above, and is especially 2-pyrrolidinoethoxy, 2-morpholinoethoxy, 3-morpholinopropoxy, 1-methyl-piperidin-3-ylmethoxy, 3-(N-methylpiperazino)propoxy or 2-(1-imidazolyl)ethoxy.

In a 5 or 6 membered carbocyclic or heterocyclic ring comprising 0, 1, 2 or 3 heteroatoms selected from N, O and S, and formed by two adjacent substituents together with the benzene ring, the ring may be further substituted, e.g. by C₁-C₈alkyl, C₁-C₈alkoxy, haloC₁-C₈alkyl, hydroxy, amino, substituted amino, C₁-C₈alkoxy, halogen, carboxy, C₁-C₈alkoxycarbonyl, carbamoyl, cyano, or oxo. The two adjacent substituents forming such a ring are preferably propylene, butylene, 1-aza-2-propylidene, 3-aza-1-propylidene, 1,2-diaza-2-propylidene, 2,3-diaza-1-propylidene, 1-oxapropylene, 1-oxapropylidene, methylenedioxy, difluoromethylene-dioxy, 2-aza-1-oxopropylene, 2-aza-2-methyl-1-oxopropylene, 1-aza-2-oxopropylene, 2-aza-1,1-dioxo-1-thiapropylene or the corresponding butylene derivatives forming a 6 membered ring.

Salts are especially the pharmaceutically acceptable salts of compounds of formula I.

Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient.

The compounds of formula I have valuable pharmacological properties, as described hereinbefore and hereinafter.

In formula I the following significances are preferred independently, collectively or in any combination or sub-combination.

### A)

R₀ is hydrogen
R₁ is hydrogen or
R₂ is hydrogen
R₃ is SO₂N(R₁₂)R₁₃ wherein R₁₂ is hydrogen or C₁-C₇alkyl and R₁₃ is hydrogen, C₁-C₇alkyl, C₁-C₇alkoxy-C₁-C₇alkyl, di-C₁-C₇alkylamino-C₁-C₇alkyl, hydroxy-C₁-C₇alkyl;
R₄ is hydrogen

### R₅ is Br or Cl

R₆ is hydrogen
R₇ is hydrogen; C₁-C₇alkoxy; carbamoyl unsubstituted or substituted by loweralkyl; 5 or 6 member heterocycl comprising 1, 2 or 3 N or O atoms unsubstituted or substituted by di-C₁-C₇alkyl-amino, C₁-C₇alkyl, hydroxy, 5 or 6 member heterocycl comprising 1, 2 or 3 N or O atoms unsubstituted or substituted by C₁-C₇alkyl; 5 or 6 member heterocycloxy comprising 1, 2 or 3 N or O ring atoms unsubstituted or substituted by C₁-C₇alkyl; heterocycl-C₁-C₇alkyloxy wherein heterocycl is a 5 or 6 member heterocycl comprising 1, 2 or 3 N or O ring atoms unsubstituted or substituted by hydroxy or C₁-C₇alkyl;
R₈ is hydrogen; halogen; C1-C7alkoxy; carbamoyl unsubstituted or substituted by C₁-C₇alkyl; heterocycl-C₁-C₇alkyloxy wherein heterocycl is a 5 or 6 member heterocycl comprising 1, 2 or 3 N or O ring atoms unsubstituted or substituted by C₁-C₇alkyl, hydroxy; 5 or 6 member heterocycl comprising 1, 2 or 3 N or O atoms unsubstituted or substituted once or twice by a substituent independently selected from hydroxy, C₁-C₇alkoxy-C₁-C₇alkyl, C₁-C₇alkyl, aminocarbonyl and C₁-C₇alkylamino; 5 or 6 member heterocycloxy comprising 1 or 2 N ring atoms unsubstituted or substituted 1 to 5 times by C₁-C₇alkyl or di-C₁-C₇alkylamino; 10 member bi-cyclic-heterocycle comprising 1 to 3 heteroatoms selected from N or O;
R₉ is hydrogen;
R₁₀ is C₁-C₇alkoxy;

### B)

R₀ is hydrogen
R₁ is hydrogen or
R₂ is hydrogen
R₃ is SO₂N(R₁₂)R₁₃ wherein R₁₂ is hydrogen or C₁-C₇alkyl and R₁₃ is hydrogen, C₁-C₇alkyl, C₁-C₇alkoxy-C₁-C₇alkyl, di-C₁-C₇alkylamino-C₁-C₇alkyl, hydroxy-C₁-C₇alkyl;
R₄ is hydrogen

### R₅ is Br or Cl

R₆ is hydrogen
R₇ is hydrogen;
R₈ is hydrogen; halogen; C1-C7alkoxy; carbamoyl unsubstituted or substituted by C₁-C₇alkyl; heterocycl-C₁-C₇alkyloxy wherein heterocycl is a 5 or 6 member heterocycl comprising 1, 2 or 3 N or O ring atoms unsubstituted or substituted by C₁-C₇alkyl, hydroxy; 5 or 6 member heterocycl comprising 1, 2 or 3 N or O atoms unsubstituted or substituted once or twice by a substituent independently selected from hydroxy, C₁-C₇alkoxy-C₁-C₇alkyl, C₁-C₇alkyl, aminocarbonyl and C₁-C₇alkylamino; 5 or 6 member heterocycloxy comprising 1 or 2 N ring atoms unsubstituted or substituted 1 to 5 times by C₁-C₇alkyl or di-C₁-C₇alkylamino; 10 member bi-cyclic-heterocycle comprising 1 to 3 heteroatoms selected from N or O;
R₉ is hydrogen;
R₁₀ is C₁-C₇alkoxy;

More preferred are the following meanings, independently, collectively or in any combination or sub-combination:

| | | | | |
|---|---|---|---|---|
| R0 | 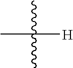 | | | |
| | H | | | |
| R1 | 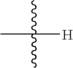 | 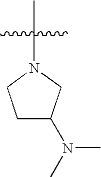 | 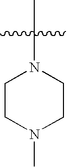 | 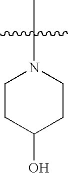 |
| | H | C6H13N2 | C5H11N2 | C5H10NO |
| R2 | 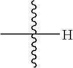 | | | |
| | H | | | |
| R3 | 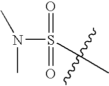 | 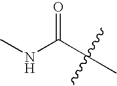 | 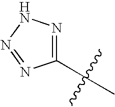 | 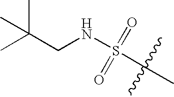 |
| | SO2NMe2 | CONHMe | [symbol] | [symbol] |
| | 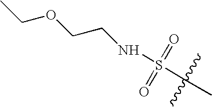 | 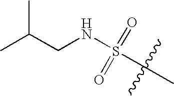 | 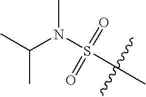 | 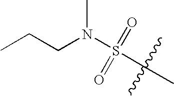 |
| | [symbol] | [symbol] | [symbol] | [symbol] |
| | 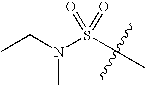 | 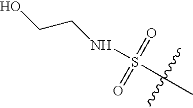 | 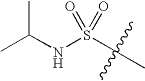 | 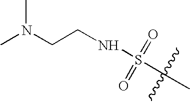 |
| | [symbol] | [symbol] | [symbol] | [symbol] |
| R2 & R3 | 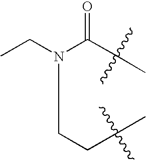 | 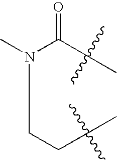 | 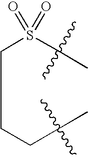 | 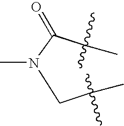 |
| | C5H9NO | [symbol] | [symbol] | [symbol] |
| R4 | 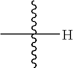 | | | |
| | H | | | |
| R5 | 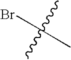 | 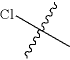 | | |
| | Br | Cl | | |
| R6 | 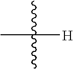 | | | |
| | H | | | |
| R7 | 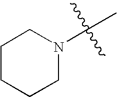 | 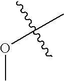 | 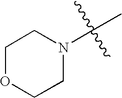 | 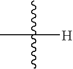 |
| | Piperidin-1-yl | OMe | N-Morpholino | H |
| | 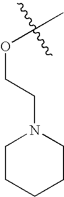 | 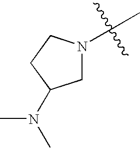 | 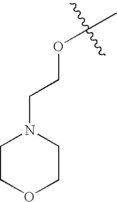 | 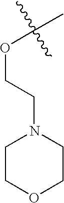 |
| | C7H14NO | C6H13N2 | C6H12NO2 | C6H12NO2 |
| | 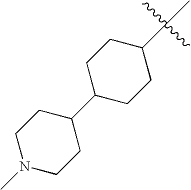 | 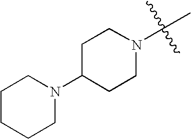 | 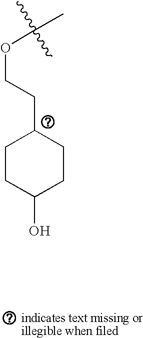 | 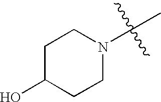 |
| | [symbol] | [symbol] | [symbol] | C5H10NO |
| R8 | 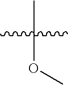 | 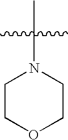 | 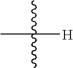 | 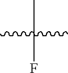 |
| | OMe | N-Morpholino | H | F |
| | 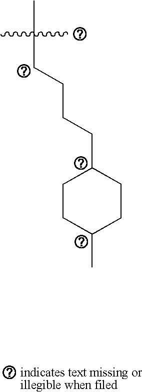 | 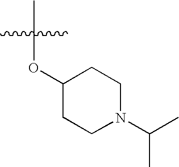 | 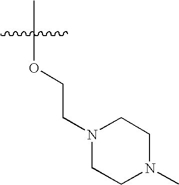 | 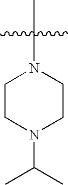 |
| | C8H11H2O | [symbol] | [symbol] | [symbol] |
| | 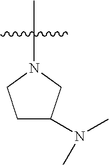 | 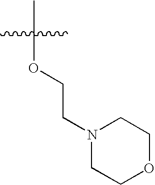 | 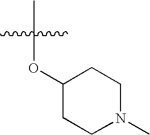 | 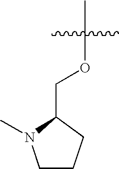 |
| | C6H13N2 | C6H12NO2 | C6H12NO | C6H12NO |
| | 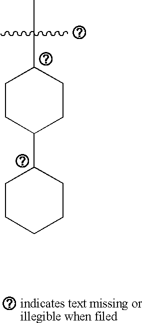 | 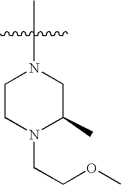 | 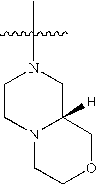 | 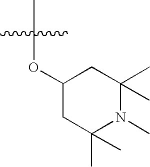 |
| | C6H13N2 | [symbol] | [symbol] | [symbol] |
| | C6H13N2 | | | |
| R7 & R8 | 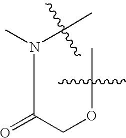 | | | |
| | C8H5NO2 | | | |
| R9 | 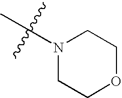 | 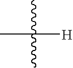 | 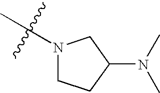 | |
| | N-Morpholino | H | C6H12N2 | |
| R10 | 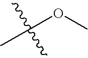 | 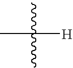 | | |
| | OMe | H | | |
| R11 | 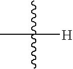 | 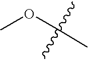 | | |
| | H | OMe | | |

| | | | |
|---|---|---|---|
| R0 | | | |
| R1 | 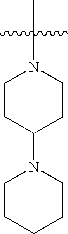 | | |
| | [symbol] | | |
| R2 | | | |
| R3 | 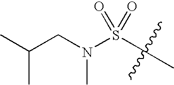 | 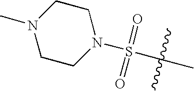 | 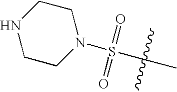 |
| | C5H12NO2S | C5H11N2O2S | C4H9N2O2S |
| | 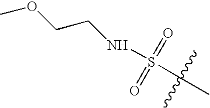 | 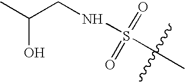 | 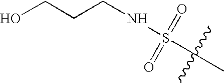 |
| | [symbol] | [symbol] | [symbol] |
| | 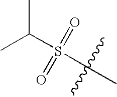 | | |
| | C3H7O2S | | |
| R2 & R3 | | | |
| R4 | | | |
| R5 | | | |
| R6 | | | |
| R7 | 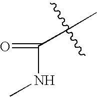 | 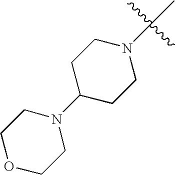 | 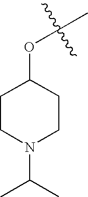 |
| | CONHMe | [symbol] | [symbol] |
| | 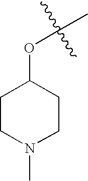 | 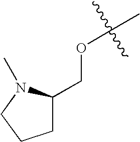 | 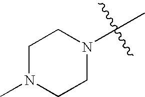 |
| | C6H12NO | C6H12NO | C5H11N2 |
| R8 | 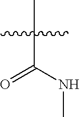 | 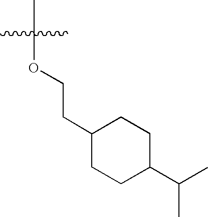 | 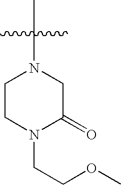 |
| | CONHMe | [symbol] | [symbol] |
| | 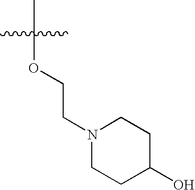 | 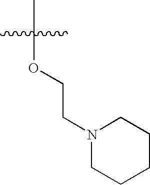 | 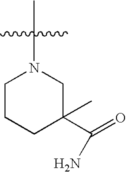 |
| | C7H14NO2 | C7H14NO | C7H13N2O |
| | 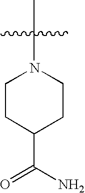 | 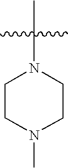 | 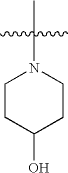 |
| | [symbol] | C5H11N2 | C5H10NO |
| | 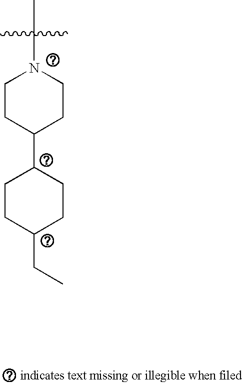 | 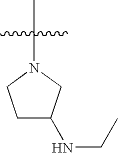 | 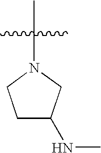 |
| | C11H22N2 | [symbol] | C5H11N2 |
| R7 & R8 | | | |
| R9 | | | |
| R10 | | | |
| R11 | | | |

[symbol]

indicates data missing or illegible when filed

Most preferred as compounds of the formula I are those wherein the substituents have the meaning given in the Examples.

The present invention also provides a process for the production of a compound of formula I, comprising reacting a compound of formula II

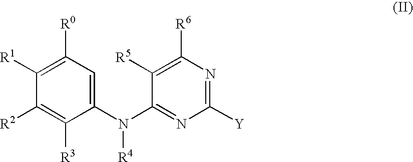

wherein R⁰, R¹, R², R³, R⁴, R⁵, and R⁶ are as defined above, and Y is a leaving group, preferably halogen such as bromide, iodine, or in particular chloride;
with a compound of formula III

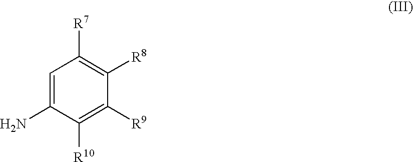

wherein R⁷, R⁸, R⁹ and R¹⁰ are as defined above;
and, if desired, converting a compound of formula I, wherein the substituents have the meaning as defined above, into another compound of formula I as defined;
and recovering the resulting compound of formula I in free from or as a salt, and, when required, converting the compound of formula I obtained in free form into the desired salt, or an obtained salt into the free form.

The reaction can be carried out in a manner known per se, the reaction conditions being dependent especially on the reactivity of the leaving group Y and the reactivity of the amino group in the aniline of formula III, usually in the presence of a suitable solvent or diluent or of a mixture thereof and, if necessary, in the presence of an acid or a base, with cooling or, preferably, with heating, for example in a temperature range from approximately −30° C. to approximately +150° C., especially approximately from 0° C. to +100° C., preferably from room temperature (approx. +20° C.) to +80° C., in an open or closed reaction vessel and/or in the atmosphere of an inert gas, for example nitrogen.

If one or more other functional groups, for example carboxy, hydroxy or amino, are or need to be protected in a compound of formula II or III, because they should not take part in the reaction, these are such groups as are usually used in the synthesis of peptide compounds, cephalosporins and penicillins, as well as nucleic acid derivatives and sugars.

The protecting groups may already be present in precursors and should protect the functional groups concerned against unwanted secondary reactions, such as substitution reaction or solvolysis. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove.

Salts of a compound of formula I with a salt-forming group may be prepared in a manner known per se. Acid addition salts of compounds of formula I may thus be obtained by treatment with an acid or with a suitable anion exchange reagent.

Salts can usually be converted to compounds in free form, e.g. by treating with suitable basic, agents, for example with alkali metal carbonates, alkali metal hydrogencarbonates, or alkali metal hydroxides, typically potassium carbonate or sodium hydroxide.

Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

It should be emphasized that reactions analogous to the conversions mentioned in this chapter may also take place at the level of appropriate intermediates.

The compounds of formula I, including their salts, are also obtainable in the form of hydrates, or their crystals can include for example the solvent used for crystallization (present as solvates).

The compound of formula II used as starting materials may be obtained by reacting a compound of formula IV

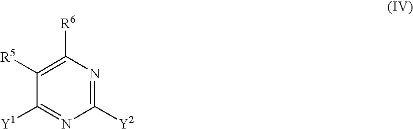

with a compound of formula V

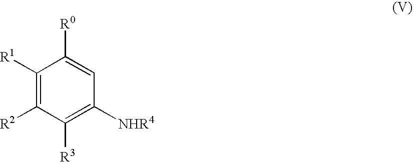

wherein R¹, R², R³, R⁴, R⁵ and R⁶ are as defined above, and Y¹ and Y² are identical or different leaving groups as defined above for Y. The reaction conditions are those mentioned above for the reaction of a compound of formula II with a compound of formula III.

The compounds of formula IV and V are known or may be produced in accordance with known procedures.

The compounds of formula I and their pharmaceutically acceptable salts exhibit valuable pharmacological properties when tested in vitro in cell-free kinase assays and in cellular assays, and are therefore useful as pharmaceuticals. In particular, the compounds of the invention are inhibitors of Focal Adhesion kinase, and are useful as pharmaceuticals to treat conditions caused by a malfunction of signal cascades connected with Focal Adhesion Kinase, in particular tumors as described hereinbelow.

Focal Adhesion Kinase (FAK) is a key enzyme in the Integrin-mediated outside-in signal cascade (D. Schlaepfer et al., Prog Biophys Mol Biol 1999, 71, 435-478). Interaction between cells and extracellular matrix (ECM) proteins is transduced as intracellular signals important for growth, survival and migration through cell surface receptors, integrins. FAK plays an essential role in these integrin-mediated outside-in signal cascades. The trigger in the signal transduction cascade is the autophosphorylation of Y397. Phosphorylated Y397 is a SH2 docking site for Src family tyrosine kinases. The bound c-Src kinase phosphorylates other tyrosine residues in FAK. Among them, phosphorylated Y925 becomes a binding site for the SH2 site of Grb2 small adaptor protein. This direct binding of Grb2 to FAK is one of the key steps for the activation of down stream targets such as the Ras-ERK2/MAP kinase cascade.

The inhibition of endogenous FAK signalling results in reduced motility and in some cases induces cell death. On the other hand, enhancing FAK signalling by exogenous expression increases cell motility and transmitting a cell survival signal from ECM. In addition FAK is overexpressed in invasive and metastatic epithelial, mesenchymal, thyroid and prostate cancers. Consequently, an inhibitor of FAK is likely to be a drug for anti-tumor growth and metastasis. The compounds of the invention are thus indicated, for example, to prevent and/or treat a vertebrate and more particularly a mammal, affected by a neoplastic disease, in particular breast tumor, cancer of the bowel (colon and rectum), stomach cancer and cancer of the ovary and prostate, non-small cell lung cancer, small cell lung cancer, cancer of liver, melanoma, bladder tumor and cancer of head and neck.

The relation between FAK inhibition and immuno-system is described e.g. in G. A. van Seventer et al., Eur. J. Immunol. 2001, 31, 1417-1427. Therefore, the compounds of the invention are, for example, useful to prevent and/or treat a vertebrate and more particularly a mammal, affected by immune system disorders, diseases or disorders mediated by T lymphocytes, B lymphocytes, mast cells and/or eosinophils e.g. acute or chronic rejection of organ or tissue allo- or xenografts, atherosclerosis, vascular occlusion due to vascular injury such as angioplasty, restenosis, hypertension, heart failure, chronic obstructive pulmonary disease, CNS disease such as Alzheimer disease or amyotrophic lateral sclerosis, cancer, infectious disease such as AIDS, septic shock or adult respiratory distress syndrome, ischemia/reperfusion injury e.g. myocardial infarction, stroke, gut ischemia, renal failure or hemorrhage shock, or traumatic shock. The agent of the invention are also useful in the treatment and/or prevention of acute or chronic inflammatory diseases or disorders or autoimmune diseases e.g. rheumatoid arthritis, osteoarthritis, systemic lupus erythematosus, Hashimoto's thyroiditis, multiple sclerosis, myasthenia gravis, diabetes (type I and II) and the disorders associated with therewith, respiratory diseases such as asthma or inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis.

Compounds of the invention are active in a FAK assay system as described in the Examples, and show an inhibition IC₅₀ in the range of 1 nM to 100 nM.

Some of the compounds of the invention exhibit also ZAP-70 (zeta chain-associated protein of 70 kD) protein tyrosine kinase inhibiting activity. ZAP-70 protein tyrosine kinase interaction of the agents of the invention may be demonstrated by their ability to prevent phosphorylation of e.g. LAT-11 (linker for activation of T cell) by human ZAP-70 protein tyrosine kinase in aqueous solution, as described in the Examples. The compounds of the invention are thus also indicated for the prevention or treatment of disorders or diseases where ZAP-70 inhibition play a role.

Compounds of the invention are active in a ZAP-70 assay system as described in the Examples, and show an inhibition IC₅₀ in the range of 1 μM to 10 μM.

Compounds of the present invention are also good inhibitors of the IGF-IR (insulin like growth factor receptor 1) and are therefore useful in the treatment of IGF-1R mediated diseases for example such diseases include proliferative diseases, such as tumours, like for example breast, renal, prostate, colorectal, thyroid, ovarian, pancreas, neuronal, lung, uterine and gastro-intestinal tumours as well as osteosarcomas and melanomas. The efficacy of the compounds of the invention as inhibitors of IGF-IR tyrosine kinase activity can be demonstrated using a cellular “Capture ELISA”. In this assay the activity of the compounds of the invention against Insulin-like growth factor I (IGF-I) induced autophosphorylation of the IGF-IR is determined.

The compounds of the present invention also exhibit powerful inhibition of the tyrosine kinase activity of anaplastic lymphoma kinase (ALK) and the fusion protein of NPM-ALK. This protein tyrosine kinase results from a gene fusion of nucleophosmin (NPM) and the anaplastic lymphoma kinase (ALK), rendering the protein tyrosine kinase activity of ALK ligand-independent. NPM-ALK plays a key role in signal transmission in a number of hematopoetic and other human cells leading to hematological and neoplastic diseases, for example in anaplastic large-cell lymphoma (ALCL) and non-Hodgkin's lymphomas (NHL), specifically in ALK+NHL or Alkomas, in inflammatory myofibroblastic tumors (IMT) and neuroblastomas. (Duyster J et al. 2001 Oncogene 20, 5623-5637). In addition to NPM-ALK, other gene fusions have been identified in human hematological and neoplastic diseases; mainly TPM3-ALK (a fusion of nonmuscle tropomyosin with ALK).

The inhibition of ALK tyrosine kinase activity can be demonstrated using known methods, for example using the recombinant kinase domain of the ALK in analogy to the VEGF-R kinase assay described in J. Wood et al. Cancer Res. 60, 2178-2189 (2000). In vitro enzyme assays using GST-ALK protein tyrosine kinase are performed in 96-well plates as a filter binding assay in 20 mM Tris.HCl, pH=7.5, 3 mM MgCl₂, 10 mM MnCl₂, 1 mM DTT, 0.1 μCi/assay (=30 μl) [γ-³³P]-ATP, 2 μM ATP, 3 μg/ml poly (Glu, Tyr 4:1) Poly-EY (Sigma P-0275), 1% DMSO, 25 ng ALK enzyme. Assays are incubated for 10 min at ambient temperature. Reactions are terminated by adding 50 μl of 125 mM EDTA, and the reaction mixture is transferred onto a MAIP Multiscreen plate (Millipore, Bedford, Mass., USA), previously wet with methanol, and rehydrated for 5 min with H₂O. Following washing (0.5% H₃PO₄), plates are counted in a liquid scintillation counter. IC₅₀ values are calculated by linear regression analysis of the percentage inhibition. Compared with the control without inhibitor, the compounds of formula I inhibit the enzyme activity by 50% (IC₅₀), for example in a concentration of from 0.001 to 0.5 μM, especially from 0.01 to 0.1 μM.

The compounds of formula I potently inhibit the growth of human NPM-ALK overexpressing murine BaF3 cells (DSMZ Deutsche Sammlung von Mikroorganismen und Zellkulturen GmbH, Braunschweig, Germany). The expression of NPM-ALK is achieved by transfecting the BaF3 cell line with an expression vector PClneo™ (Promega Corp., Madison Wis., USA) coding for NPM-ALK and subsequent selection of G418 resistant cells. Non-transfected BaF3 cells depend on IL-3 for cell survival. In contrast NPM-ALK expressing BaF3 cells (named BaF3-NPM-ALK hereinafter) can proliferate in the absence of IL-3 because they obtain proliferative signal through NPM-ALK kinase. Putative inhibitors of the NPM-ALK kinase therefore abolish the growth signal and result in antiproliferative activity. The antiproliferative activity of putative inhibitors of the NPM-ALK kinase can however be overcome by addition of IL-3 which provides growth signals through an NPM-ALK independent mechanism. [For an analogous cell system using FLT3 kinase see E Weisberg et al. Cancer Cell; 1, 433-443 (2002)]. The inhibitory activity of the compounds of formula I is determined, briefly, as follows: BaF3-NPM-ALK cells (15,000/microtitre plate well) are transferred to 96-well microtitre plates. The test compounds [dissolved in dimethyl sulfoxide (DMSO)] are added in a series of concentrations (dilution series) in such a manner that the final concentration of DMSO is not greater than 1% (v/v). After the addition, the plates are incubated for two days during which the control cultures without test compound are able to undergo two cell-division cycles. The growth of the BaF3-NPM-ALK cells is measured by means of Yopro™ staining [T Idziorek et al. J. Immunol. Methods; 185: 249-258 (1995)]: 25 μl of lysis buffer consisting of 20 mM sodium citrate, pH 4.0, 26.8 mM sodium chloride, 0.4% NP40, 20 mM EDTA and 20 mM is added to each well. Cell lysis is completed within 60 min at room temperature and total amount of Yopro bound to DNA is determined by measurement using the Cytofluor II 96-well reader (PerSeptive Biosystems) with the following settings: Excitation (nm) 485/20 and Emission (nm) 530/25.

IC₅₀ values are determined by a computer-aided system using the formula:

IC₅₀=[(ABSₜₑₛₜ−ABSₛₜₐᵣₜ)/(ABS_{control}−ABSₛₜₐᵣₜ)]×100. (ABS=absorption)

The IC₅₀ value in those experiments is given as that concentration of the test compound in question that results in a cell count that is 50% lower than that obtained using the control without inhibitor. The compounds of formula I exhibit inhibitory activity with an IC₅₀ in the range from approximately 0.01 to 1 μM.

The antiproliferative action of the compounds of formula I can also be determined in the human KARPAS-299 lymphoma cell line (DSMZ Deutsche Sammlung von Mikroorganismen und Zellkulturen GmbH, Braunschweig, Germany) [described in W G Dirks et al. Int. J. Cancer 100, 49-56 (2002)] using the same methodology described above for the BaF3-NPM-ALK cell line. The compounds of formula I exhibit inhibitory activity with an IC₅₀ in the range from approximately 0.01 to 1 μM.

The action of the compounds of formula I on autophosphorylation of the ALK can be determined in the human KARPAS-299 lymphoma cell line by means of an immunoblot as described in W G Dirks et al. Int. J. Cancer 100, 49-56 (2002). In that test the compounds of formulas exhibit an IC₅₀ of approximately from 0.001 to 1 μM.

For the above uses in the treatment of neoplastic diseases and immune system disorders the required dosage will of course vary depending on the mode of administration, the particular condition to be treated and the effect desired. In general, satisfactory results are indicated to be obtained systemically at daily dosages of from about 0.1 to about 100 mg/kg body weight. An indicated daily dosage in the larger mammal, e.g. humans, is in the range from about 0.5 mg to about 2000 mg, conveniently administered, for example, in divided doses up to four times a day or in retard form.

The compounds of the invention may be administered by any conventional route, in particular parenterally, for example in the form of injectable solutions or suspensions, enterally, preferably orally, for example in the form of tablets or capsules, topically, e.g. in the form of lotions, gels, ointments or creams, or in a nasal or a suppository form. Pharmaceutical compositions comprising a compound of the invention in association with at least one pharmaceutical acceptable carrier or diluent may be manufactured in conventional manner by mixing with a pharmaceutically acceptable carrier or diluent. Unit dosage forms for oral administration contain, for example, from about 0.1 mg to about 500 mg of active substance. Topical administration is e.g. to the skin. A further form of topical administration is to the eye.

The pharmaceutical compositions of the present invention are prepared in a manner known per se, for example by means of conventional mixing, granulating, coating, dissolving or lyophilizing processes.

Preference is given to the use of solutions of the active ingredient, and also suspensions or dispersions, especially isotonic aqueous solutions, dispersions or suspensions which, for example in the case of lyophilized compositions comprising the active ingredient alone or together with a carrier, for example mannitol, can be made up before use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving and lyophilizing processes. The said solutions or suspensions may comprise viscosity-increasing agents, typically sodium carboxymethylcellulose, carboxymethylcellulose, dextran, polyvinylpyrrolidone, or gelatins, or also solubilizers, e.g. Tween 80® (polyoxyethylene(20)sorbitan mono-oleate).

Suspensions in oil comprise as the oil component the vegetable, synthetic, or semi-synthetic oils customary for injection purposes. In respect of such, special mention may be made of liquid fatty acid esters that contain as the acid component a long-chained fatty acid having from 8 to 22, especially from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brassidic acid or linoleic acid, if desired with the addition of antioxidants, for example vitamin E, β-carotene or 3,5-di-tert-butyl-4-hydroxytoluene. The alcohol component of these fatty acid esters has a maximum of 6 carbon atoms and is a monovalent or polyvalent, for example a mono-, di- or trivalent, alcohol, for example methanol, ethanol, propanol, butanol or pentanol or the isomers thereof, but especially glycol and glycerol. As fatty acid esters, therefore, the following are mentioned: ethyl-oleate, isopropyl myristate, isopropyl palmitate, “Labrafil M 2375” (polyoxyethylene glycerol), “Labrafil M 1944 CS” (unsaturated polyglycolized glycerides prepared by alcoholysis of apricot kernel oil and consisting of glycerides and polyethylene glycol ester), “Labrasol” (saturated polyglycolized glycerides prepared by alcoholysis of TCM and consisting of glycerides and polyethylene glycol ester; all available from Gattefossé, France), and/or “Miglyol 812” (triglyceride of saturated fatty acids of chain length C₈ to C₁₂ from Hüls AG, Germany), but especially vegetable oils such as cottonseed oil, almond oil, olive oil, castor oil, sesame oil, soybean oil and more especially groundnut oil.

The manufacture of injectable preparations is usually carried out under sterile conditions, as is the filling, for example, into ampoules or vials, and the sealing of the containers.

Pharmaceutical compositions for oral administration can be obtained, for example, by combining the active ingredient with one or more solid carriers, if desired granulating a resulting mixture, and processing the mixture or granules, if desired or necessary, by the inclusion of additional excipients, to form tablets or tablet cores.

Suitable carriers are especially fillers, such as sugars, for example lactose, saccharose, mannitol or sorbitol, cellulose preparations, and/or calcium phosphates, for example tricalcium phosphate or calcium hydrogen phosphate, and also binders, such as starches, for example corn, wheat, rice or potato starch, methylcellulose, hydroxypropyl methylcellulose, sodium carboxymethylcellulose, and/or polyvinylpyrrolidone, and/or, if desired, disintegrators, such as the above-mentioned starches, also carboxymethyl starch, crosslinked polyvinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Tablet cores can be provided with suitable, optionally enteric, coatings through the use of, inter alia, concentrated sugar solutions which may comprise gum arabic, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes or pigments may be added to the tablets or tablet coatings, for example for identification purposes or to indicate different-doses of active ingredient.

Pharmaceutical compositions for oral administration also include hard capsules consisting of gelatin, and also soft, sealed capsules consisting of gelatin and a plasticizer, such as glycerol or sorbitol. The hard capsules may contain the active ingredient in the form of granules, for example in admixture with fillers, such as corn starch, binders, and/or glidants, such as talc or magnesium stearate, and optionally stabilizers. In soft capsules, the active ingredient is preferably dissolved or suspended in suitable liquid excipients, such as fatty oils, paraffin oil or liquid polyethylene glycols or fatty acid esters of ethylene or propylene glycol, to which stabilizers and detergents, for example of the polyoxyethylene sorbitan fatty acid ester type, may also be added.

Pharmaceutical compositions suitable for rectal administration are, for example, suppositories that consist of a combination of the active ingredient and a suppository base. Suitable suppository bases are, for example, natural or synthetic triglycerides, paraffin hydrocarbons, polyethylene glycols or higher alkanols.

For parenteral administration, aqueous solutions of an active ingredient in water-soluble form, for example of a water-soluble salt, or aqueous injection suspensions that contain viscosity-increasing substances, for example sodium carboxymethylcellulose, sorbitol and/or dextran, and, if desired, stabilizers, are especially suitable. The active ingredient, optionally together with excipients, can also be in the form of a lyophilizate and can be made into a solution before parenteral administration by the addition of suitable solvents.

Solutions such as are used, for example, for parenteral administration can also be employed as infusion solutions.

Preferred preservatives are, for example, antioxidants, such as ascorbic acid, or microbicides, such as sorbic acid or benzoic acid.

The compounds of the invention may be administered as the sole active ingredient or together with other drugs useful against neoplastic diseases or useful in immunomodulating regimens. For example, the agents of the invention may be used in accordance with the invention in combination with pharmaceutical compositions effective in various diseases as described above, e.g. with cyclophosphamide, 5-fluorouracil, fludarabine, gemcitabine, cisplatinum, carboplatin, vincristine, vinblastine, etoposide, irinotecan, paclitaxel, docetaxel, rituxan, doxorubicine, gefitinib, or imatinib; or also with cyclosporins, rapamycins, ascomycins or their immunosuppressive analogs, e.g. cyclosporin A, cyclosporin G, FK-506, sirolimus or everolimus, corticosteroids, e.g. prednisone, cyclophosphamide, azathioprene, methotrexate, gold salts, sulfasalazine, antimalarials, brequinar, leflunomide, mizoribine, mycophenolic acid, mycophenolate, mofetil, 15-deoxyspergualine, immuno-suppressive monoclonal antibodies, e.g. monoclonal antibodies to leukocyte receptors, e.g. MHC, CD2, CD3, CD4, CD7, CD25, CD28, CD40, CD45, CD58, CD80, CD86, CD152, CD137, CD154, ICOS, LFA-1, VLA-4 or their ligands, or other immunomodulatory compounds, e.g. CTLA41g.

In accordance with the foregoing, the present invention also provides:

(1) A compound of the invention for use as a pharmaceutical;
(2) a compound of the invention for use as a FAK inhibitor, an ALK inhibitor and/or ZAP-70 inhibitor, for example for use in any of the particular indications hereinbefore set forth;
(3) a pharmaceutical composition, e.g. for use in any of the indications herein before set forth, comprising a compound of the Invention as active ingredient together with one or more pharmaceutically acceptable diluents or carriers;
(4) a method for the treatment of any particular indication set forth hereinbefore in a subject in need thereof which comprises administering an effective amount of a compound of the invention or a pharmaceutical composition comprising same;
(5) the use of a compound of the invention for the manufacture of a medicament for the treatment or prevention of a disease or condition in which FAK, ALK and/or ZAP-70 activation plays a role or is implicated;
(6) the method as defined above under (4) comprising co-administration, e.g. concomitantly or in sequence, of a therapeutically effective amount of a compound of the invention and one or more further drug substances, said further drug substance being useful in any of the particular indications set forth hereinbefore;
(7) a combination comprising a therapeutically effective amount of a compound of the invention and one or more further drug substances, said further drug substance being useful in any of the particular indications set forth hereinbefore;
(8) use of a compound of the invention for the manufacture of a medicament for the treatment or prevention of a disease which responds to inhibition of the anaplastic lymphoma kinase;
(9) the use according to (8), wherein the disease to be treated is selected from anaplastic large-cell lymphoma, non-Hodgkin's lymphomas, inflammatory myofibroblastic tumors and neuroblastomas;
(10) the use according to (8) or (9), wherein the compound is or a pharmaceutically acceptable salt of any one of the examples;
(11) a method for the treatment of a disease which responds to inhibition of the anaplastic lymphoma kinase, especially a disease selected from anaplastic large-cell lymphoma, non-Hodgkin'S lymphomas, inflammatory myofibroblastic tumors and neuroblastomas, comprising administering an effective amount of a compound of the invention or a pharmaceutically acceptable salt thereof.

Additionally preferred a compound according to the present invention that is useful as herein before described is a compound specifically mentioned in the examples.

Additional specifically preferred compounds according to the present invention that are useful either as FAK inhibitor, as ALK inhibitor or for inhibition of both and which may be prepared essentially according to the methods described hereinbefore are the following:
2-{5-Bromo-2-[5-(3-dimethylamino-pyrrolidin-1-yl-2-methoxy-phenylamino]-pyrimidin-4-ylamino}-N-methyl-benzenesulfonamide,
2-{5-Bromo-2-[2-methoxy-5-(2-morpholin-4-yl-ethoxy)-phenylamino]-pyrimidin-4-ylamino}-N-isopropyl-benzenesulfonamide,
7-[2-(4-[1,4′]Bipiperidinyl-1′-yl-2-methoxy-phenylamino)-5-chloro-pyrimidin-4-ylamino]-2-methyl-2,3-dihydro-isoindol-1-one,
2-[5-Chloro-2-(2-methoxy-4-morpholin-4-yl-phenylamino)-pyrimidin-4-ylamino]-5-(4-hydroxy-piperidin-1-yl)-N-methyl-benzamide,
5-[1,4′]Bipiperidinyl-1′-yl-2-[5-chloro-2-(2-methoxy-5-morpholin-4-yl-phenylamino)-pyrimidin-4-ylamino]-N-methyl-benzamide,
2-[2-(4-[1,4′]Bipiperidinyl-1′-yl-2-methoxy-phenylamino)-5-chloro-pyrimidin-4-ylamino]-N-isobutyl-benzenesulfonamide,
2-{5-Chloro-2-[2-methoxy-4-(4-methyl-piperazin-1-yl)-phenylamino]-pyrimidin-4-ylamino}-N-isobutyl-benzenesulfonamide,
2-[5-Chloro-2-(2-methoxy-5-morpholin-4-yl-phenylamino)-pyrimidin-4-ylamino]-5-(4-hydroxy-piperidin-1-yl)-N-methyl-benzamide,
2-[2-(5-[1,4′]Bipiperidinyl-1′-yl-2-methoxy-phenylamino)-5-bromo-pyrimidin-4-ylamino]-N-isopropyl-benzenesulfonamide,
1-{4-[5-Chloro-4-(2-isobutylsulfamoyl-phenylamino)-pyrimidin-2-ylamino]-3-methoxy-phenyl}-piperidine-4-carboxylic acid amide,
4-[5-Chloro-4-(2-isobutylsulfamoyl-phenylamino)-pyrimidin-2-ylamino]-3-methoxy-N-methyl-benzamide,
2-{5-Chloro-2-[4-(4-hydroxy-piperidin-1-yl)-2-methoxy-phenylamino]-pyrimidin-4-ylamino}-N-isobutyl-benzenesulfonamide,
3-[5-Chloro-4-(2 isobutylsulfamoyl-phenylamino)-pyrimidin-2-ylamino]-4-methoxy-N-methyl-benzamide,
5-Chloro N²-(2-methoxy-4-morpholin-4-yl-phenyl)-N⁴-[2-(2H-tetrazol-5-yl)-phenyl]-pyrimidine-2,4-diamine,
2-{5-Chloro-2-[4-((S)-3-dimethylamino-pyrrolidin-1-yl)-2-methoxy-phenylamino]-pyrimidin-4-ylamino}-N-isobutyl-benzenesulfonamide,
2-{5-Chloro-2-[2-methoxy-4-(1-methyl-piperidin-4-yloxy)-phenylamino]-pyrimidin-4-ylamino}-N-isobutyl-benzenesulfonamide,
7-{5-Chloro-2-[4-((S)-3-dimethylamino-pyrrolidin-1-yl)-2-methoxy-phenylamino]-pyrimidin-4-ylamino}-2-methyl-2,3-dihydro-isoindol-1-one,
2-{5-Chloro-2-[4-((S)-3-dimethylamino-pyrrolidin-1-yl)-2-methoxy-phenylamino]-pyrimidin-4-ylamino}-N-methyl-5-(4-methyl-piperazin-1-yl)-benzamide,
1-{4-[5-Chloro-4-(2-methylcarbamoyl-phenylamino)-pyrimidin-2-ylamino]-3-methoxy-phenyl}-3-methyl-piperidine-3-carboxylic acid amide,
1-{4-[5-Chloro-4-(2-methyl-3-oxo-2,3-dihydro-1H-isoindol-4-ylamino)-pyrimidin-2-ylamino]-3-methoxy-phenyl}-3-methyl-piperidine-3-carboxylic acid amide,
1-{4-[5-Chloro-4-(2-isobutylsulfamoyl-phenylamino)-pyrimidin-2-ylamino]-3-methoxy-phenyl}-3-methyl-piperidine-3-carboxylic acid amide,
2-{5-Chloro-2-[5-(3-dimethylamino-pyrrolidin-1-yl)-2-methoxy-phenylamino]-pyrimidin-4-ylamino}-N-methyl-5-(4-methyl-piperazin-1-yl)-benzamide,
7-{5-Chloro-2-[2-methoxy-4-(1-methyl-piperidin-4-yloxy)-phenylamino]-pyrimidin-4-ylamino}-2-methyl-2,3-dihydro-isoindol-1-one,
2-[5-Bromo-2-(2,5-dimethoxy-4-morpholin-4-yl-phenylamino)-pyrimidin-4-ylamino]-N-methyl-benzenesulfonamide,
2-{5-Bromo-2-[5-(4-hydroxy-piperidin-1-yl)-2-methoxy-phenylamino]-pyrimidin-4-ylamino}-N-methyl-benzenesulfonamide,
2-[5-Chloro-2-(2-methoxy-5-morpholin-4-yl-phenylamino)-pyrimidin-4-ylamino]-N-isobutyl-benzenesulfonamide,
2-{5-Chloro-2-[2-methoxy-5-(4-methyl-piperazin-1-yl)-phenylamino]-pyrimidin-4-ylamino}-N-isobutyl-benzenesulfonamide,
2-[2-(5-[1,4′]Bipiperidinyl-1′-yl-2-methoxy-phenylamino)-5-chloro-pyrimidin-4-ylamino]-N-isobutyl-benzenesulfonamide,
2-{5-Chloro-2-[5-((S)-3-dimethylamino-pyrrolidin-1-yl)-2-methoxy-phenylamino]-pyrimidin-4-ylamino}-N-isobutyl-benzenesulfonamide,
1-{4-[5-Chloro-4-(2-methyl-3-oxo-2,3-dihydro-1H-isoindol-4-ylamino)-pyrimidin-2-ylamino]-3-methoxy-phenyl}-piperidine-4-carboxylic acid amide,
2-[5-Chloro-2-(2-methoxy-4-morpholin-4-yl-phenylamino)-pyrimidin-4-ylamino]-5-((S)-3-dimethylamino-pyrrolidin-1-yl)-N-methyl-benzamide,
7-{5-Chloro-2-[4-(4-isopropyl-piperazin-1-yl)-2-methoxy-phenylamino]-pyrimidin-4-ylamino}-2-methyl-2,3-dihydro-isoindol-1-one,
2-[5-Chloro-2-(2-methoxy-4-morpholin-4-yl-phenylamino)-pyrimidin-4-ylamino]-N-(2,2-dimethyl-propyl)-benzenesulfonamide,
2-{5-Chloro-2-[2-methoxy-4-(4-methyl-piperazin-1-yl)-phenylamino]-pyrimidin-4-ylamino}-N-(2,2-dimethyl-propyl)-benzenesulfonamide,
3-[5-Chloro-4-(2-isobutylsulfamoyl-phenylamino)-pyrimidin-2-ylamino]-4-methoxy-benzamide,
2-[5-Bromo-2-(2,4-dimethoxy-5-morpholin-4-yl-phenylamino)-pyrimidin-4-ylamino]-N-methyl-benzenesulfonamide,
2-{5-Bromo-2-[5-(1-isopropyl-piperidin-4-yloxy)-2-methoxy-phenylamino]-pyrimidin-4-ylamino}-N-methyl-benzenesulfonamide,
7-(5-Chloro-2-{2-methoxy-4-[2-(4-methyl-piperazin-1-yl)-ethoxy]-phenylamino}-pyrimidin-4-ylamino)-2-methyl-2,3-dihydro-isoindol-1-one,
2-(5-Chloro-2-{2-methoxy-4-[4-(4-methyl-piperazin-1-yl)-piperidin-1-yl]-phenylamino}-pyrimidin-4-ylamino)-N-isobutyl-benzenesulfonamide,
(S)-1-{4-[5-Chloro-4-(2-methyl-3-oxo-2,3-dihydro-1H-isoindol-4-ylamino)-pyrimidin-2-ylamino]-3-methoxy-phenyl}-3-methyl-piperidine-3-carboxylic acid amide,
(S)-1-{4-[5-Chloro-4-(2-methylcarbamoyl-phenylamino)-pyrimidin-2-ylamino]-3-methoxy-phenyl}-3-methyl-piperidine-3-carboxylic acid amide,
7-[5-Chloro-2-(2,4-dimethoxy-phenylamino)-pyrimidin-4-ylamino]-2-methyl-2,3-dihydro-isoindol-1-one,
2-(5-Bromo-2-{2-methoxy-5-[4-(4-methyl-piperazin-1-yl)-piperidin-1-yl]-phenylamino}-pyrimidin-4-ylamino)-N-methyl-benzenesulfonamide,
2-{5-Bromo-2-[5-(4-hydroxy-piperidin-1-yl)-2-methoxy-phenylamino]-pyrimidin-4-ylamino}-N-isopropyl-benzenesulfonamide,
2-{5-Bromo-2-[2-methoxy-5-(1-methyl-piperidin-4-yloxy)-phenylamino]-pyrimidin-4-ylamino}-N-isopropyl-benzenesulfonamide,
2-{5-Bromo-2-[5-(1-isopropyl-piperidin-4-yloxy)-2-methoxy-phenylamino]-pyrimidin-4-ylamino}-N-isopropyl-benzenesulfonamide,
7-{5-Chloro-2-[2-methoxy-4-(2-morpholin-4-yl-ethoxy)-phenylamino]-pyrimidin-4-ylamino}-2-methyl-2,3-dihydro-isoindol-1-one,
7-{5-Chloro-2-[2-methoxy-5-(2-morpholin-4-yl-ethoxy)-phenylamino]-pyrimidin-4-ylamino}-2-methyl-2,3-dihydro-isoindol-1-one,
7-{5-Chloro-2-[4-(1-isopropyl-piperidin-4-yloxy)-2-methoxy-phenylamino]-pyrimidin-4-ylamino}-2-methyl-2,3-dihydro-isoindol-1-one,
2-{5-Bromo-2-[5-(3-dimethylamino-pyrrolidin-1-yl)-2-methoxy-phenylamino]-pyrimidin-4-ylamino}-N-isopropyl-benzenesulfonamide,
2-{5-Bromo-2-[2-methoxy-5-(4-methyl-piperazin-1-yl)-phenylamino]-pyrimidin-4-ylamino}-N-isopropyl-benzenesulfonamide,
2-(5-Bromo-2-{2-methoxy-5-[4-(4-methyl-piperazin-1-yl)-piperidin-1-yl]-phenylamino}-pyrimidin-4-ylamino)-N-isopropyl-benzenesulfonamide,
7-{5-Chloro-2-[2-methoxy-4-(1,2,2,6,6-pentamethyl-piperidin-4-yloxy)-phenylamino]-pyrimidin-4-ylamino}-2-methyl-2,3-dihydro-isoindol-1-one,
1-{4-[5-Chloro-4-(2-methyl-3-oxo-2,3-dihydro-1H-isoindol-4-ylamino)-pyrimidin-2-ylamino]-3-methoxy-phenyl}-piperidine-3-carboxylic acid amide,
2-{5-Chloro-2-[2-methoxy-4-(1,2,2,6,6-pentamethyl-piperidin-4-yloxy)-phenylamino]-pyrimidin-4-ylamino}-N-isobutyl-benzenesulfonamide,
(R)-1-{4-[5-Chloro-4-(2-methylcarbamoyl-phenylamino)-pyrimidin-2-ylamino]-3-methoxy-phenyl}-3-methyl-piperidine-3-carboxylic acid amide,
(R)-1-{4-[5-Chloro-4-(2-methyl-3-oxo-2,3-dihydro-1H-isoindol-4-ylamino)-pyrimidin-2-ylamino]-3-methoxy-phenyl}-3-methyl-piperidine-3-carboxylic acid amide,
2-{5-Chloro-2-[2-methoxy-4-((R)-1-methyl-pyrrolidin-2-ylmethoxy)-phenylamino]-pyrimidin-4-ylamino}-N-isobutyl-benzenesulfonamide,
2-{5-Chloro-2-[2-methoxy-4-((S)-1-methyl-pyrrolidin-2-ylmethoxy)-phenylamino]-pyrimidin-4-ylamino}-N-isobutyl-benzenesulfonamide,
2-{5-Bromo-2-[2-methoxy-5-(2-piperidin-1-yl-ethoxy)-phenylamino]-pyrimidin-4-ylamino}-N-methyl-benzenesulfonamide,
2-(5-Bromo-2-{5-[2-(4-hydroxy-piperidin-1-yl)-ethoxy]-2-methoxy-phenylamino}-pyrimidin-4-ylamino)-N-methyl-benzenesulfonamide,
5-Chloro-N⁴-(1,1-dioxo-1λ⁶-thiochroman-8-yl)-N²-(2-methoxy-4-morpholin-4-yl-phenyl)-pyrimidine-2,4-diamine,
2-[5-Chloro-2-(2-methoxy-4-morpholin-4-yl-phenylamino)-pyrimidin-4-ylamino]-N-(2-hydroxy-ethyl)-benzenesulfonamide,
2-[5-Chloro-2-(2-methoxy-4-morpholin-4-yl-phenylamino)-pyrimidin-4-ylamino]-N-(2-methoxy-ethyl)-benzenesulfonamide,
7-{5-Chloro-2-[2-methoxy-4-(2-piperidin-1-yl-ethoxy)-phenylamino]-pyrimidin-4-ylamino}-2-methyl-2,3-dihydro-isoindol-1-one,
2-[5-Chloro-2-(2-methoxy-4-morpholin-4-yl-phenylamino)-pyrimidin-4-ylamino]-N-((R)-2-hydroxy-propyl)-benzenesulfonamide,
2-[5-Chloro-2-(2-methoxy-4-morpholin-4-yl-phenylamino)-pyrimidin-4-ylamino]-N-(3-hydroxy-propyl)-benzenesulfonamide,
2-[5-Chloro-2-(2-methoxy-4-morpholin-4-yl-phenylamino)-pyrimidin-4-ylamino]-N-((S)-2-hydroxy-propyl)-benzenesulfonamide,
2-{5-Bromo-2-[2-methoxy-5-(4-morpholin-4-yl-piperidin-1-yl)-phenylamino]-pyrimidin-4-ylamino}-N-isopropyl-benzenesulfonamide,
7-(5-Chloro-2-{2-methoxy-4-[(S)-4-(2-methoxy-ethyl)-3-methyl-piperazin-1-yl]-phenylamino}-pyrimidin-4-ylamino)-2-methyl-2,3-dihydro-isoindol-1-one,
7-(5-Chloro-2-{2-methoxy-4-[(R)-4-(2-methoxy-ethyl)-3-methyl-piperazin-1-yl]-phenylamino}-pyrimidin-4-ylamino)-2-methyl-2,3-dihydro-isoindol-1-one,
5-Chloro-N²-[4-((S)-3-dimethylamino-pyrrolidin-1-yl)-2-methoxy-phenyl]-N⁴-(1,1-dioxo-1λ⁶-thiochroman-8-yl)-pyrimidine-2,4-diamine,
5-Chloro-N⁴-(1,1-dioxo-1λ⁶-thiochroman-8-yl)-N²-{2-methoxy-4-[4-(4-methyl-piperazin-1-yl)-piperidin-1-yl]-phenyl}-pyrimidine-2,4-diamine,
2-{5-Bromo-2-[2-methoxy-5-(4-morpholin-4-yl-piperidin-1-yl)-phenylamino]-pyrimidin-4-ylamino}-N-methyl-benzenesulfonamide,
2-[5-Bromo-2-(4-fluoro-2-methoxy-5-morpholin-4-yl-phenylamino)-pyrimidin-4-ylamino]-N-methyl-benzenesulfonamide,
4-[5-Chloro-4-(1,1-dioxo-1λ⁶-thiochroman-8-ylamino)-pyrimidin-2-ylamino]-3-methoxy-N-methyl-benzamide,
2-{5-Bromo-2-[2-methoxy-5-((S)-1-methyl-pyrrolidin-2-ylmethoxy)-phenylamino]-pyrimidin-4-ylamino}-N-methyl-benzenesulfonamide,
2-{5-Bromo-2-[2-methoxy-5-((R)-1-Methyl-pyrrolidin-2-ylmethoxy)-phenylamino]-pyrimidin-4-ylamino}-N-methyl-benzenesulfonamide,
2-{5-Bromo-2-[2,4-dimethoxy-5-(2-morpholin-4-yl-ethoxy)-phenylamino]-pyrimidin-4-ylamino}-N-methyl-benzenesulfonamide,
2-[5-Chloro-2-(2-methoxy-4-morpholin-4-yl-phenylamino)-pyrimidin-4-ylamino]-N-isopropyl-N-methyl-benzenesulfonamide,
2-[5-Chloro-2-(2-methoxy-4-morpholin-4-yl-phenylamino)-pyrimidin-4-ylamino]-N-methyl-N-propyl-benzenesulfonamide,
7-(5-Chloro-2-{4-[2-(4 isopropyl-piperazin-1-yl)-ethoxy]-2-methoxy-phenylamino}-pyrimidin-4-ylamino)-2-methyl-2,3-dihydro-isoindol-1-one,
2-{5-Bromo-2-[2-methoxy-5-(2-morpholin-4-yl-ethoxy)-phenylamino]-pyrimidin-4-ylamino}-N,N-dimethyl-benzenesulfonamide,
2-[5-Bromo-2-(2,4-dimethoxy-5-morpholin-4-yl-phenylamino)-pyrimidin-4-ylamino]-N-isopropyl-benzenesulfonamide,
2-[5-Chloro-2-(2-methoxy-4-morpholin-4-yl-phenylamino)-pyrimidin-4-ylamino]-N-(2-dimethylamino-ethyl)-benzenesulfonamide,
5-Chloro-N²-(2-methoxy-4-morpholin-4-yl-phenyl)-N⁴-[2-(4-methyl-piperazine-1-sulfonyl)-phenyl]-pyrimidine-2,4-diamine,
2-[5-Chloro-2-(2-methoxy-4-morpholin-4-yl-phenylamino)-pyrimidin-4-ylamino]-N-(2-ethoxy-ethyl)-benzenesulfonamide,
2-[5-Bromo-2-(7-methoxy-4-methyl-3-oxo-3,4-dihydro-2H-benzo[1,4]oxazin-6-ylamino)-pyrimidin-4-ylamino]-N-methyl-benzenesulfonamide,
2-[5-Bromo-2-(2-methoxy-5-morpholin-4-yl-phenylamino)-pyrimidin-4-ylamino]-N,N-dimethyl-benzenesulfonamide,
2-[5Bromo-2-(2-methoxy-5-morpholin-4-yl-phenylamino)-pyrimidin-4-ylamino]-N,N-dimethyl-benzenesulfonamide,
2-{5-Chloro-2-[2-methoxy-4-(4-methyl-piperazin-1-yl)-phenylamino]-pyrimidin-4-ylamino}-N-methyl-N-propyl-benzenesulfonamide,
2-[5-Bromo-2-(2-methoxy-5-piperidin-1-yl-phenylamino)-pyrimidin-4-ylamino]-N-methyl-benzenesulfonamide,
2-[5-Bromo-2-(2-methoxy-5-piperidin-1-yl-phenylamino)-pyrimidin-4-ylamino]-N-isopropyl-benzenesulfonamide,
7-{5-Chloro-2-[4-((R)-3-dimethylamino-pyrrolidin-1-yl)-2-methoxy-phenylamino]-pyrimidin-4-ylamino}-2-methyl-2,3-dihydro-isoindol-1-one,
5-Chloro-N²-(2-methoxy-4-morpholin-4-yl-phenyl)-N⁴-[2-(piperazine-1-sulfonyl)-phenyl]-pyrimidine-2,4-diamine,
2-[5-Chloro-2-(2-methoxy-4-morpholin-4-yl-phenylamino)-pyrimidin-4-ylamino]-N-isobutyl-N-methyl-benzenesulfonamide,
2-[5-Chloro-2-(2-methoxy-4-morpholin-4-yl-phenylamino)-pyrimidin-4-ylamino]-N-ethyl-N-methyl-benzenesulfonamide,
2-{5-Chloro-2-[2-methoxy-4-(4-methyl-piperazin-1-yl)-phenylamino]-pyrimidin-4-ylamino}-N-isobutyl-N-methyl-benzenesulfonamide,
2-{5-Chloro-2-[2-methoxy-4-(4-methyl-piperazin-1-yl)-phenylamino]-pyrimidin-4-ylamino}-N-ethyl-N-methyl-benzenesulfonamide,
7-(5-Chloro-2-{4-[2-(4-hydroxy-piperidin-1-yl)-ethoxy]-2-methoxy-phenylamino}-pyrimidin-4-ylamino)-2-methyl-2,3-dihydro-isoindol-1-one,
2-(5-Bromo-2-{2-methoxy-5-[4-(4-methyl-piperazin-1-yl)-piperidin-1-yl]-phenylamino}pyrimidin-4-ylamino)-N,N-dimethyl-benzenesulfonamide,
8-{5-Chloro-2-[2-methoxy-4-(4-methyl-piperazin-1-yl)-phenylamino]-pyrimidin-4-ylamino}-2-methyl-3,4-dihydro-2H-isoquinolin-1-one,
8-[5-Chloro-2-(2-methoxy-4-morpholin-4-yl-phenylamino)-pyrimidin-4-ylamino]-2-methyl-3,4-dihydro-2H-isoquinolin-1-one,
8-(5-Chloro-2-{2-methoxy-4-[4-(4-methyl-piperazin-1-yl)-piperidin-1-yl]-phenylamino}-pyrimidin-4-ylamino)-2-methyl-3,4-dihydro-2H-isoquinolin-1-one,
8-[2-(4-[1,4′]Bipiperidinyl-1′-yl-2-methoxy-phenylamino)-5-chloro-pyrimidin-4-ylamino]-2-methyl-3,4-dihydro-2H-isoquinolin-1-one,
8-{5-Chloro-2-[4-(4-hydroxy-piperidin-1-yl)-2-methoxy-phenylamino]-pyrimidin-4-ylamino}-2-methyl-3,4-dihydro-2H-isoquinolin-1-one,
8-{5-Chloro-2-[4-(4-isopropyl-piperazin-1-yl)-2-methoxy-phenylamino]-pyrimidin-4-ylamino}-2-methyl-3,4-dihydro-2H-isoquinolin-1-one,
7-(5-Chloro-2-{2-methoxy-4-[3-(4-methyl-piperazin-1-yl)-propoxy]-phenylamino}-pyrimidin-4-ylamino)-2-methyl-2,3-dihydro-isoindol-1-one,
8-{5-Chloro-2-[4-((S)-3-dimethylamino-pyrrolidin-1-yl)-2-methoxy-phenylamino]-pyrimidin-4-ylamino}-2-methyl-3,4-dihydro-2H-isoquinolin-1-one,
8-{5-Chloro-2-[4-((R)-3-dimethylamino-pyrrolidin-1-yl)-2-methoxy-phenylamino]-pyrimidin-4-ylamino}-2-methyl-3,4-dihydro-2H-isoquinolin-1-one,
8-[5-Chloro-2-((S)-4-hexahydro-pyrazino[2,1-c][1,4]oxazin-8-yl-2-methoxy-phenylamino)-pyrimidin-4-ylamino]-2-methyl-3,4-dihydro-2H-isoquinolin-1-one,
8-[5-Chloro-2-((R)-4-hexahydro-pyrazino[2,1-c][1,4]oxazin-8-yl-2-methoxy-phenylamino)-pyrimidin-4-ylamino]-2-methyl-3,4-dihydro-2H-isoquinolin-1-one,
8-[5-Chloro-2-(2-methoxy-4-morpholin-4-yl-phenylamino)-pyrimidin-4-ylamino]-2-ethyl-3,4-dihydro-2H-isoquinolin-1-one,
8-(5-Chloro-2-{2-methoxy-4-[4-(4-methyl-piperazin-1-yl)-piperidin-1-yl]-phenylamino}-pyrimidin-4-ylamino)-2-ethyl-3,4-dihydro-2H-isoquinolin-1-one,
2-[5-Chloro-2-(2-methoxy-5-morpholin-4-yl-phenylamino)-pyrimidin-4-ylamino]-N-methyl-5-(4-methyl-piperazin-1-yl)-benzamide,
5-[1,4′]Bipiperidinyl-1′-yl-2-[5-chloro-2-(2-methoxy-4-morpholin-4-yl-phenylamino)-pyrimidin-4-ylamino]-N-methyl-benzamide,
2-[5-Bromo-2-(2-methoxy-5-morpholin-4-yl-phenylamino)-pyrimidin-4-ylamino]-N-isopropyl-benzenesulfonamide,
5-Chloro-N²-{4-[4-(4-ethyl-piperazin-1-yl)-piperidin-1-yl]-2-methoxy-phenyl}-N⁴-[2-(propane-2-sulfonyl)-phenyl]-pyrimidine-2,4-diamine,
2-{5-Chloro-2-[4-((S)-3-ethylamino-pyrrolidin-1-yl)-2-methoxy-phenylamino]-pyrimidin-4-ylamino}-N-isopropyl-benzenesulfonamide,
2-{5-Chloro-2-[4-((R)-3-ethylamino-pyrrolidin-1-yl)-2-methoxy-phenylamino]-pyrimidin-4-ylamino}-N-isopropyl-benzenesulfonamide,
2-{5-Chloro-2-[2-methoxy-4-((S)-3-methylamino-pyrrolidin-1-yl)-phenylamino]-pyrimidin-4-ylamino}-N-isopropyl-benzenesulfonamide,
2-{5-Chloro-2-[2-methoxy-4-((R)-3-methylamino-pyrrolidin-1-yl)-phenylamino]-pyrimidin-4-ylamino}-N-isopropyl-benzenesulfonamide,
2-{5-Chloro-2-[4-((R)-3-dimethylamino-pyrrolidin-1-yl)-2-methoxy-phenylamino]-pyrimidin-4-ylamino}-N-isopropyl-benzenesulfonamide,
2-{5-Chloro-2-[4-((S)-3-dimethylamino-pyrrolidin-1-yl)-2-methoxy-phenylamino]-pyrimidin-4-ylamino}-N-isopropyl-benzenesulfonamide,
2-{5-Chloro-2-[2-ethoxy-4-(4-methyl-piperazin-1-yl)-phenylamino]-pyrimidin-4-ylamino}-N-isopropyl-benzenesulfonamide,
2-{5-Chloro-2-[2-isopropoxy-4-(4-methyl-piperazin-1-yl)-phenylamino]-pyrimidin-4-ylamino}-N-isopropyl-benzenesulfonamide,
2-{5-Chloro-2-[2-cyclopropylmethoxy-4-(4-methyl-piperazin-1-yl)-phenylamino]-pyrimidin-4-ylamino}-N-isopropyl-benzenesulfonamide

### EXAMPLES

### Abbreviations

AcOH=acetic acid, ALK=anaplastic lymphoma kinase, ATP=adenosine 5′-triphosphate, brine=saturated sodium chloride solution, BSA=bovine serum albumin, DIAD=diisopropyl azodicarboxylate, DIPCDI=N,N′-diisopropylcarbodiimid, DMAP=4-dimethylaminopyridine, DMF=N,N-dimethylformamide, DTT=1,4-dithio-D,L-threitol, EDTA=ethylene diamine tetraacetic acid, Et=ethyl, EtOAc=ethyl acetate, EtOH=ethanol, Eu-PT66=LANCE™ europium-W1024-labelled anti-phosphotyrosine antibody (Perkin Elmer), FAK=Focal Adhesion Kinase, FRET=fluorescence resonance energy transfer, HEPES=N-2-hydroxyethyl-piperazine-N′-2-ethanesulfonic acid, HOAt=1-hydroxy-7-azabenzotriazole, Me=methyl, RT-PCR=reverse transcription polymerase chain reaction, SA-(SL)APC=Streptavidin conjugated to SuperLight™ allophycocyanin (Perkin Elmer), subst.=substituted, TBTU=O-(benzotriazol-1-yl)-N,N,N′,N′-tetramethylammonium tetrafluoroborate, THF=tetrahydrofuran.

### HPLC Conditions

Column: YMC CombiScreen ODS-A (5 um, 12 nm), 50×4.6 mm I.D.
Flow rate: 2.0 ml/min

### Eluent: A) TFA/water (0.1/100), B) TFA/acetonitrile (0.1/100)

Gradient: 5-100% B (0-5 min)

### Detection: UV at 215 nm

### Example 1

### Preparation of 4-[5-Chloro-4-(2-isobutylsulfamoyl-phenylamino)-pyrimidin-2-ylamino]-3-methoxy-N-methyl-benzamide

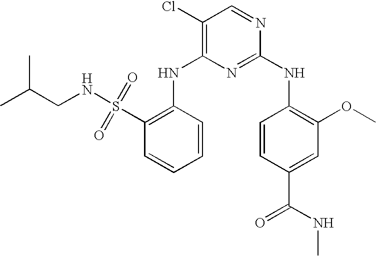

To a solution of 2-(2,5-dichloro-pyrimidin-4-ylamino)-N-isobutyl-benzenesulfonamide (200 mg, 0.56 mmol) and 4-amino-3-methoxy-N-methyl-benzamide (121 mg, 0.672 mmol) in AcOH (4 mL), 1N HCl/EtOH (1 ml) is added at room temperature. The mixture is heated at 100° C. for 15 h. The solvent is evaporated, and the residue is purified by reverse phase HPLC to give the title product. MS (ESI) m/z 519, HPLC retention time 3.18 min.

### Example 2

The following 2-[5-chloro-2-(subst. phenylamino)-pyrimidin-4-ylamino]-N-isobutyl-benzene-sulfonamides are prepared from 2-(2,5-dichloro-pyrimidin-4-ylamino)N-isobutyl-benzenesulfonamide and the corresponding aniline following the procedure of Example 1:

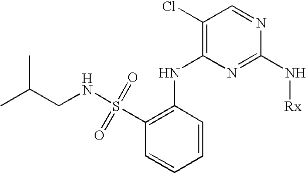

| Expl | | Mass (ESI) | NMR (400 MHz) δ (ppm) or |
|---|---|---|---|
| No. | Rx | or Rf (solvent) | HPLC Retention time (min) |
| 3 | 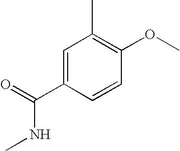 | 519 | 2.93 |
| 4 | 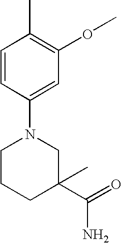 | MS 602, 604 | CDCl3: 0.75 (s, 3 H), 0.76 (s, 3 H), 1.83-1.72 (m, 1 H), 1.99-1.85 (m, 1 H), 2.17-2.09 (m, 1 H), 2.71-2.59 (m, 2 H), 2.59 (d, 1 H), 2.65 (dd, 1 H), 3.56 (d, 1 H), 3.90 (s, 3 H), 4.50 (t, 1 H), 6.52 (dd, 1 H), 6.61 (d, 1 H), 7.31 (s, 1 H), 7.62-7.57 (m, 1 H), 7.86-7.74 (m, 1 H), 7.97 (dd, 1 H), 8.09 (d, 1 H), 8.15 (s, 1 H), 8.46 (d, 1 H), 9.00 (s, 1 H). |
| 5 | 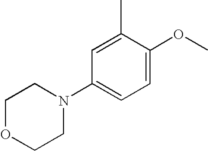 | 547 [M + 1]⁺ | DMSO-D6: 0.70 (d, 6 H), 1.55-1.62 (m, 1 H), 2.55-2.58 (m, 2 H), 2.79-2.82 (m, 4 H), 3.61-3.63 (m, 4 H), 3.76 (s, 3 H), 6.61 (dd, 1 H), 6.93 (d, 1 H), 7.25 (t, 1 H), 7.47-7.52 (m, 2 H), 7.80 (d, 1 H), 7.99 (brs, 1 H), 8.13 (s, 1 H), 8.34 (d, 1 H), 9.22 (s, 1 H) |
| 6 | 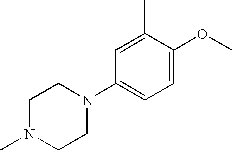 | 560 [M + 1]⁺ | DMSO-D6: 0.74 (d, 6 H), 1.55-1.62 (m, 1 H), 2.19 (s, 3 H), 2.34 (brs, 4 H), 2.55-2.59 (m, 2 H), 2.85 (brs, 4 H), 3.75 (s, 3 H), 6.60 (dd, 1 H), 6.91 (d, 1 H), 7.23 (t, 1 H), 7.44 (s, 1 H), 7.50 (t, 2 H), 7.80 (d, 1 H), 7.98 (t, 1 H), 6.13 (s, 1 H), 8.27 (s, 1 H), 8.37 (d, 1 H), 9.24 (s, 1 H) |
| 7 | 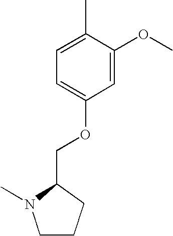 | 575 [M + 1]⁺ | DMSO-D6: 0.75 (d, 6 H), 1.55-1.73 (m, 4 H), 1.94-2.00 (m, 1 H), 2.23 (brs, 1 H), 2.40 (s, 3 H), 2.56 (t, 2 H), 2.99 (brs, 1 H), 3.83-3.88 (m, 1 H), 3.96-4.02 (m, 1 H), 6.48 (dd, 1 H), 6.64 (d, 1 H), 7.22 ( t, 1 H), 7.42-7.49 (m, 3 H), 7.77 (d, 1 H), 7.94 (t, 1 H), 8.17 (s, 1 H), 8.23 (s, 1 H), 8.42 (d, 1 H), 9.32 (s, 1 H) |
| 8 | 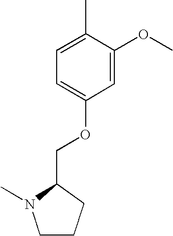 | 575 [M + 1]⁺ | DMSO-D6: 0.75 (d, 6 H), 1.55-1.73 (m, 4 H), 1.94-2.00 (m, 1 H), 2.23 (brs, 1 H), 2.40 (s, 3 H), 2.56 (t, 2 H), 2.99 (brs, 1 H), 3.83-3.88 (m, 1 H), 3.96-4.02 (m, 1 H), 6.48 (dd, 1 H). 6.64 (d, 1 H), 7.22 (t, 1 H), 7.42-7.49 (m, 3 H), 7.77 (d, 1 H), 7.94 (t, 1 H), 8.17 (s, 1 H), 8.23 (s, 1 H), 8.42 (d, 1 H), 9.32 (s, 1 H) |
| 9 | 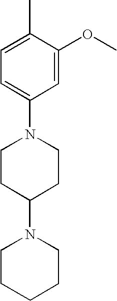 | 628 [M + 1]⁺ | CDCl₃: 0.75 (d,. 6 H), 1.46-1.49 (m, 2 H), 1.54-1.65 (m, 5 H), 1.68-1.78 (m, 2 H), 1.93 (d, 2 H), 2.36-2.40 (m, 1 H), 2.54-2.56 (m, 4 H), 2.65-2.75 (m, 4 H), 3.65 (d, 2 H), 3.87 (s, 3 H), 4.55 (t, 1 H), 6.43 (dd, 1 H), 6.55 (d, 1 H), 7.23 (t, 1 H), 7.31 (s, 1 H), 7.58 (t, 1 H), 7.96 (t, 2 H), 8.12 (s, 1 H), 8.46 (d, 1 H), 8.97 (s, 1 H) |
| 10 | 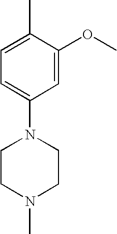 | 560 [M + 1]⁺ | CDCl₃: 0.75 (d, 6 H), 1.57-1.65 (m, 1 H), 2.37 (s, 3 H), 2.59-2.61 (m, 4 H), 2.73 (t, 2 H), 3.16-3.18 (m, 4 H), 3.87 (s, 3 H), 4.62 (br s, 1 H), 6.43 (dd, 1 H), 6.54 (d, 1 H), 7.24 (t, 1 H), 7.31 (s, 1 H), 7.57 (t, 1 H), 7.97 (dd, 2 H), 8.11 (s, 1 H), 8.46 (d, 1 H), 8.98 (s, 1 H) |
| 11 | 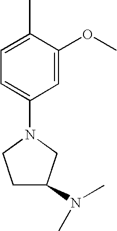 | 574 [M + 1]⁺ | CDCl₃: 0.83 (d, 6 H), 1.60-1.66 (m, 1 H), 1.92-1.99 (m, 1 H), 2.19-2.29 (m, 1 H), 2.36 (s, 6 H), 2.73 (t, 2 H), 2.84 -2.92 ( m, 1 H), 3.16 (t, 1 H), 3.31-3.37 (m, 1 H), 3.41-3.51 (m, 2 H), 3.88 (s, 3 H), 4.68 (t, 1 H), 6.06 (d, 1 H). 6.13 (d, 1 H), 7.12 (s, 1 H), 7.21 (t, 1 H), 7.54 (t, 1 H), 7.84 (d, 1 H), 7.94 (dd, 1 H), 8.07 (s, 1 H), 8.52 (d, 1 H), 9.01 (s, 1 H) |
| 12 | 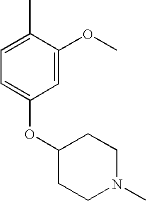 | 575 [M + 1]⁺ | CDCl₃: 0.76 (d, 6 H), 1.62-1.65 (m, 1 H), 1.80-1.89 (m, 2 H), 1.98-2.04 (m, 2 H), 2.26-2.30 (m, 2 H), 2.31 (s, 3 H), 2.71-2.76 (m, 4 H), 3.86 (s, 3 H), 4.24-4.28 (m, 1 H), 4.56 (t, 1 H), 6.40 (dd, 1 H), 6.52 (d, 1 H), 7.21-7.29 (m, 2 H), 7.57 (t, 1 H), 7.97 (dd, 2 H), 8.12 (s, 1 H), 8.44 (d, 1 H), 8.99 (s, 1 H) |
| 13 | 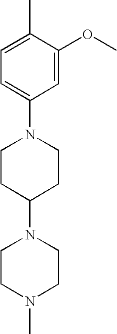 | 643 [M + 1]⁺ | CDCl₃: 0.75 (d, 6 H), 1.58-1.75 (m, 3 H), 1.96 (d, 2 H), 2.31 (d, 3 H), 2.35-2.75 (m, 13 H), 3.65 (d, 1 H), 3.86 (s, 3 H), 4.68 (br s, 1 H), 6.42 (dd, 1 H), 6.54 (d, 1 H), 7.22-7.31 (m, 2 H), 7.57 (t, 1 H), 8.10 (s, 1 H), 8.46 (d, 1 H), 8.99 (s, 1 H) |
| 14 | 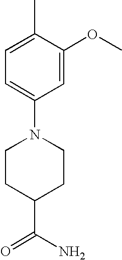 | 588 [M + 1]⁺ | DMSO-d₆: 0.74 (d, 6 H), 1.55-1.72 (m, 3 H), 1.78-1.82 (m, 2 H), 2.20-2.28 (m, 1 H), 2.55-2.80 (m, 4 H), 3.68-3.75 (m, 2 H), 3.76 (s, 3 H), 6.45 (d, 1 H), 6.63 (s, 1 H), 6.79 (s, 1 H), 7.21 (t, 1 H), 7.29 (s, 1 H), 7.37 (d, 1 H), 7.47 (t, 1 H), 7.77 (d, 1 H), 7.94 (s, 1 H), 8.15-8.20 (m, 2 H), 8.44 (brs, 1 H), 9.32 (s, 1 H) |
| 15 | 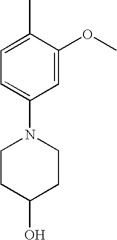 | 561 [M + 1]⁺ | DMSO-d₆: 0.74 (d, 6 H), 1.50-1.64 (m, 3 H), 1.75-1.86 (m, 2 H), 2.54-2.58 (m, 2 H), 2.74-2.82 (m, 2 H), 3.49-3.55 (m, 2 H), 3.59-3.69 (m, 1 H), 3.76 (s, 3 H), 4.64-4.69 (m, 1 H), 6.44 (d, 1 H), 6.62 (s, 1 H), 7.21 (t, 1 H), 7.36 (s, 1 H), 7.46 (t, 1 H), 7.77 (d, 1 H), 7.94 (s, 1 H), 8.10-8.17 (m, 2 H), 8.45 (brs, 1 H), 9.31 (s, 1 H) |
| 16 | 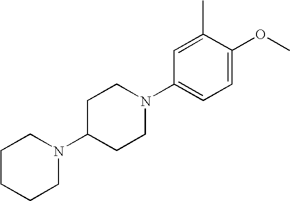 | 628 [M + 1]⁺ | CDCl₃: 0.78 (d, 6 H), 1.40-1.80 (m, 11 H), 2.23-2.30 (m, 1 H), 2.42-2.55 (m, 6 H), 2.75 (t, 2 H), 3.41 (d, 2 H), 3.85 (s, 3 H), 4.60 (t, 1 H), 6.54 (dd, 1 H), 6.78 (d, 1 H), 7.22 (t, 1 H), 7.56 (s, 1 H), 7.61 (t, 1 H), 7.92-7.99 (m, 2 H), 8.19 (s, 1 H), 8.46 (d, 1 H), 9.01 (s, 1 H) |
| 17 | 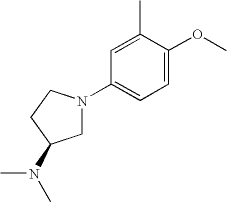 | 574 [M + 1]⁺ | CDCl₃: 0.79 (d, 6 H), 1.65 (sep, 1 H), 1.74-1.85 (m, 1 H), 2.00-2.08 (m, 1 H), 2.26 (s, 6 H), 2.74 (t, 2 H), 2.75-2.82 (m, 1 H), 2.99 (t, 1 H), 2.99-3.10 (m, 2 H), 3.32 (t, 1 H), 3.83 (s, 3 H), 4.64 (t, 1 H), 6.17 (dd, 1 H), 6.82 (d, 1 H), 7.21 (t, 1 H), 7.51-7.58 (m, 3 H), 7.95 (dd, 1 H), 8.18 (s, 1 H), 8.49 (d, 1 H), 9.01 (s, 1 H) |
| 18 | 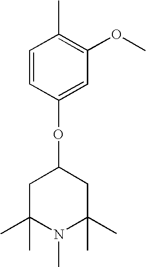 | MS: 631 | CDCl3: 0.73 (t, 6 H), 1.05-2.05 (m, 12 H), 1.35-1.5 (m, 2 H), 1.54-1.6 (m, 1 H), 1.93-2.05 (m, 2 H), 2.15-2.3 (m, 3 H), 2.58 (t, 2 H), 3.75 (s, 3 H), 4.57-4.67 (m, 1 H), 6.47-6.53 (m, 1 H), 6.6-6.66 (m, 1 H), 7.17-7.23 (m, 1 H), 7.4-7.48 (m, 2 H), 7.78 (dd, 1 H), 7.94 (dd, 1 H), 8.17 (s, 1 H), 8.25 (s, 1 H), 8.38-8.46 (m, 1 H), 9.31 (s, 1 H) |

### Example 19

### Preparation of 3-[5-Chloro-4-(2-isobutylsulfamoyl-phenylamino)-pyrimidin-2-ylamino]-4-methoxy-benzamide

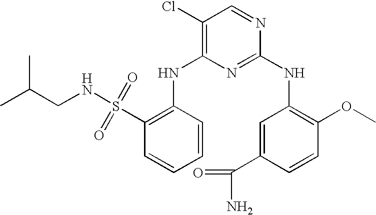

4-(2′,4′-Dimethoxyphenyl-Fmoc-aminomethyl)-phenoxy resin (1 mmol) is swelled by dichloromethane. After removing dichloromethane, the resin is treated with 20% piperidine/DMF (10 ml) at room temperature for 1 h. The solution is removed, and the resin is washed with DMF and dichloromethane. To the resin, DMF (10 ml), 4-methoxy-3-nitro-benzoic acid (394 mg, 2 mmol), PyBop (1.04 g, 2 mmol), HOBt (270 mg, 2 mmol) and DIEA (695 ul, 2 mmol) are added. After stirring the mixture at room temperature for 15 h, the solution is removed, and the resin is washed with DMF and dichloromethane. To the resin, DMF (10 ml) and tin chloride dehydrate (1.12 g, 10 mmol) are added. After stirring the mixture at 80° C. for 15 h, the solution is removed, and the resin is washed with DMF and dichloromethane. To the resin, 2-(2,5-dichloro-pyrimidin-4-ylamino)-N-isobutyl-benzenesulfonamide (750 mg, 2 mmol), 1N HCl/EtOH (2 ml) and AcOH (8 ml) are added. After stirring the mixture at 100° C. for 15 h, the resin is removed. The solution is concentrated in vacuo, and the residue is purified by reverse phase HPLC to give the title product: MS (ESI) m/z 505, HPLC retention time 2.80 min.

### Example 20

### Preparation of 1-{4-[5-Chloro-4-(2-methylcarbamoyl-phenylamino)-pyrimidin-2-ylamino]-3-methoxy-phenyl}-3-methyl-piperidine-3-carboxylic Acid Amide

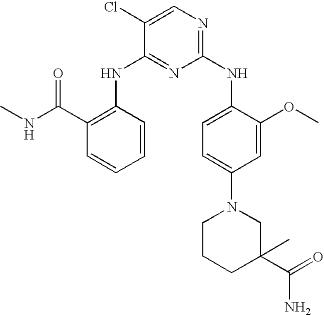

To a solution of 1-(4-amino-3-methoxy-phenyl)-3-methyl-piperidine-3-carboxylic acid amide (300 mg, 1.01 mmol) in 2-methoxyethanol (3.0 mL), 2-(2,5-dichloro-pyrimidin-4-ylamino)-N-methyl-benzamide (266.9 mg, 1.01 mmol) and 4N hydrogen chloride in ethyl acetate (1.0 mL) are added and stirred at 110° C. for 7 hours. The mixture is cooled, then poured into saturated sodium hydrogen carbonate and extracted twice with ethyl acetate. The organic layer is successively washed with water and brine, dried over magnesium sulfate, and evaporated in vacuo. The residue is purified by column chromatography to give 7-[5-Chloro-2-(2-methoxy-phenylamino)-pyrimidin-4-ylamino]-2-methyl-4-methyl-piperazin-1-yl)-2,3-dihydro-isoindol-1-one (189.8 mg) as yellow solid in 36% yield. ESI-MS (m/z): 524 [MH]⁺, ¹H-NMR (400 MHz, δ, ppm) CDCl₃: 1.24 (s, 3H), 1.33-1.18 (m, 1H), 1.81-1.70 (m, 1H), 1.99-1.83 (m, 1H), 2.16-2.07 (m, 1H), 2.59 (d, 1H), 2.66-2.59 (m, 1H), 3.04 (d, 3H), 3.53-3.46 (m, 1H), 3.56 (d, 1H), 3.90 (s, 3H), 5.45 (d, 1H), 6.18 (d, 1H), 6.61-6.57 (m, 2H), 7.10 (ddd, 1H), 7.30 (s, 1H), 7.53-7.45 (m, 2H), 7.93-7.79 (bm, 1H), 8.10 (s, 1H), 8.18 (d, 1H), 8.68 (d, 1H), 11.0 (s, 1H).

### Example 21

The following 2-[5-chloro-2-(substituted phenylamino)-pyrimidin-4-ylamino]-N-methyl-benzamide are prepared from 2-(2,5-dichloro-pyrimidin-4-ylamino)-N-methyl-benzamide and the corresponding aniline following the procedure of Example 20.

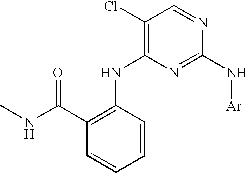

| Expl | | Rf (solvent) | |
|---|---|---|---|
| No. | Rx | Or MS (ESI) | NMR (400 MHz), δ (ppm) |
| 22 | 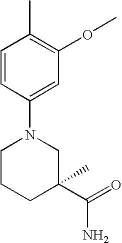 | 524 | CDCl3: 1.24 (s, 3 H), 1.33-1.18 (m, 2 H), 1.81-1.70 (m, 1 H), 1.99-1.83 (m, 1 H), 2.16-2.07 (m, 1 H), 2.59 (d, 1 H), 2.68-2.57 (m, 1 H), 3.04 (d, 3 H), 3.54-3.46 (m, 1 H), 3.56 (d, 1 H), 3.90 (s, 3 H), 5.52-5.40 (m, 1 H), 6.27-6.17 (bm, 1 H), 6.61-6.57 (m, 2 H), 7.10 (ddd, 1 H), 7.30 (s, 1 H), 7.52-7.45 (m, 2 H), 7.95-7.86 (m, 1 H), 8.10 (s, 1 H), 8.18 (d, 1 H), 8.68 (d, 1 H), 11.0 (s, 1 H). |
| 23 | 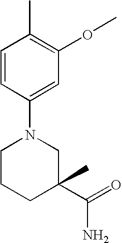 | 524 | CDCl3: 1.24 (s, 3 H), 1.33-1.18 (m, 2 H), 1.81-1.70 (m, 1 H), 1.99-1.83 (m, 1 H), 2.16-2.07 (m, 1 H), 2.59 (d, 1 H), 2.68-2.57 (m, 1 H), 3.04 (d, 3 H), 3.54-3.46 (m, 1 H), 3.56 (d, 1 H), 3.90 (s, 3 H), 5.52-5.40 (m, 1 H), 6.27-6.17 (bm, 1 H), 6.61-6.57 (m, 2 H), 7.10 (ddd, 1 H), 7.30 (s, 1 H), 7.52-7.45 (m, 2 H), 7.95-7.86 (m, 1 H), 8.10 (s, 1 H), 8.18 (d, 1 H), 8.68 (d, 1 H), 11.0 (s, 1 H). |

### Example 24

The following 2-[5-chloro-2-(subst. phenylamino)-pyrimidin-4-ylamino]-N-neopentyl-benzene-sulfonamides are prepared from 2-(2,5-dichloro-pyrimidin-4-ylamino)-N-neopentyl-benzenesulfonamide and the corresponding aniline following the procedure of Example 1:

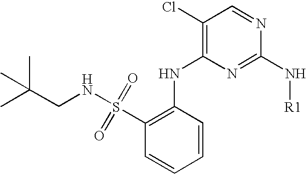

| Expl | | Mass (ESI) | HPLC Retention |
|---|---|---|---|
| No. | Rx | m/z | time (min) |
| 25 | 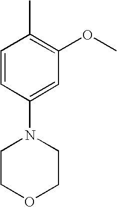 | 561 | 3.23 |
| 26 | 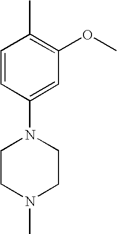 | 574 | 3.02 |

### Example 27

The following 2-[5-bromo-2-(subst. phenylamino)-pyrimidin-4-ylamino]-N-methyl-benzene-sulfonamides are prepared from 2-(2-bromo-5-chloro-pyrimidin-4-ylamino)-N-methyl-benzenesulfonamide and the corresponding aniline following the procedure of Example 1 or

### Example 20

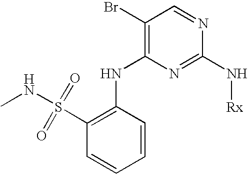

| Expl | | Mass (m/z) or | |
|---|---|---|---|
| No. | Rx | Rf (solvent) | NMR (400 MHz), δ (ppm) |
| 28 | 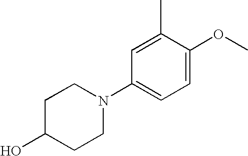 | 563 565 [M + 1]⁺ | DMSO-d6: 1:32-1.55 (m, 2 H), 1.63-1.85 (m, 2 H), 2.43 (s, 3 H), 30.9-3.58 (m, 4 H), 3.75 (s, 3 H), 4.62 (brs, 1 H), 6.56-6.72 (m, 1 H), 6.84-7.00 (m, 1 H), 7.18-7.34 (m, 1 H), 7.37-7.59 (m, 2 H), 7.71-7.87 (m, 2 H), 8.08-8.46 (m, 3 H), 9.08-9.28 (m, 1 H) |
| 29 | 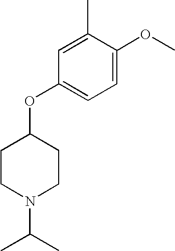 | 606 608 [M + 1]⁺ | DMSO-d6: 0.95 (d, 6 H), 1.45-1.56 (m, 2 H). 1.79-1.88 (m, 2 H), 2.22 (t, 1 H), 2.44 (s, 3 H), 2.62-2.71 (m, 4 H), 3.77 (s, 3 H), 4.00-4.07 (m, 1 H), 6.59 (dd, 1 H), 6.91 (d, 1 H), 7.27 (t, 1 H), 7.50-7.59 (m, 2 H), 7.74-7.82 (m, 2 H), 8.09 (s, 1 H), 8.34 (s, 1 H), 8.40 (s, 2 H), 9.20 (s, 1 H) |
| 30 | 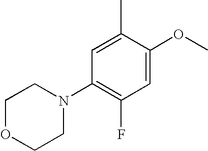 | 567 569 [M + 1] | DMSO-d6: 2.44 (s, 3 H), 2.69-2.76 (m, 4 H), 3.61-3.66 (m, 4 H), 3.75 (s, 3 H), 7.00 (d, 1 H), 7.24 (t, 1 H), 7.32 (d, 1 H), 7.45 (d, 1 H), 8.22-8.34 (m, 3 H), 9.15 (s, 1 H) |
| 31 | 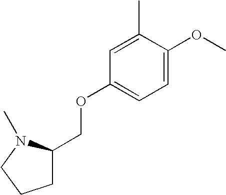 | 577 579 [M + 1] | DMSO-d6: 1.49-1.71 (m, 3 H), 1.87-1.99 (m, 1 H), 2.14-2.20 (m, 1 H), 2.31 (s, 3 H), 2.44 (s, 3 H), 2.91-2.96 (m, 1 H), 3.77 (s, 3 H), 3.79-3.84 (m, 1 H), 6.60 (dd, 1 H), 6.94 (d, 1 H), 7.26 (t, 1 H), 7.54-7.59 (m, 2 H), 7.77-7.80 (m, 2 H), 8.12 (s, 1 H), 8.35 (s, 1 H), 8.41 (d, 1 H), 9.22 (s, 1 H) |
| 32 | 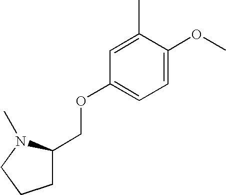 | 577 579 [M + 1] | DMSO-d6: 1.49-1.71 (m, 3 H), 1.87-1.99 (m, 1 H), 2.14-2.20 (m, 1 H), 2.31 (s, 3 H), 2.44 (s, 3 H), 2.91-2.96 (m, 1 H), 3.77 (s, 3 H), 3.79-3.84 (m, 1 H), 6.60 (dd, 1 H), 6.94 (d, 1 H), 7.26 (t, 1 H), 7.54-7.59 (m, 2 H), 7.77-7.80 (m, 2 H), 8.12 (s, 1 H), 8.35 (s, 1 H), 8.41 (d, 1 H), 9.22 (s, 1 H) |
| 33 | 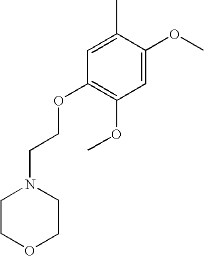 | 623 625 [M + 1] | DMSO-d6: 2.38-2.46 (m, 7 H), 2.58 (t, 2 H), 3.53 (t, 4 H), 3.78 (s, 3 H), 3.81 (s, 3 H), 3.84 (t, 2 H), 6.76 (s, 1 H), 6.72 (t, 1 H), 7.25 (s, 1 H), 7.43 (t, 1 H), 7.74-7.77 (m, 2 H), 8.25-8.27 (m, 2 H), 8.40 (d, 1 H), 9.21 (s, 1 H) |
| 34 | 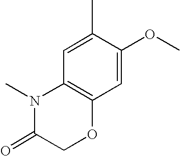 | 549 551 [M + 1] | DMSO-d6: 2.56 (s, 3 H), 3.81 (s, 3 H), 4.69 (s, 2 H), 6.87 (s, 1 H), 7.26 (t, 1 H), 7.41-7.48 (m, 2 H), 7.83 (d, 1 H), 8.33 (s, 1 H), 8.39 (d, 1 H), 8.47 (s, 1 H), 9.26 (s, 1 H) |
| 35 | 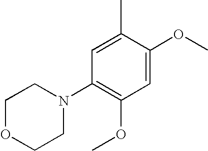 | 579 581 [M + 1]⁺ | DMSO-d₆: 2.43 (d, 3 H), 2.69-2.76 (m, 4 H), 3.59-3.67 3.67 (m, 4 H), 3.77 (s, 3 H), 3.84 (s, 3 H), 6.74 (s, 1 H), 7.08 (s, 1 H), 7.20 (t, 1 H), 7.40 (brs, 1 H), 7.74-7.80 (m, 2 H), 8.22-8.43 (m, 3 H), 9.17 (s, 1 H) |
| 36 | 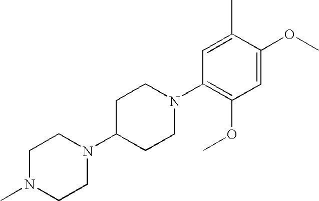 | 647, 645 [M + 1]⁺ | CDCl₃: 1.50-1.65 (m, 3 H), 1.71-1.81 (m, 2 H), 2.21-2.28 (m, 1 H), 2.30 (s, 3 H), 2.40-2.52 (m, 5 H), 2.55-2.64 (m, 7 H), 3.40 (d, 2 H), 3.84 (s, 3 H), 4.70 (br s, 1 H), 6.53 (dd, 1 H), 6.78 (d, 1 H), 7.24 (t, 1 H), 7.56 (s, 1 H), 7.60 (t, 1 H), 7.96 (dd, 1 H), 8.27 (s, 1 H), 8.44 (d, 1 H), 8.97 (s, 1 H) |
| 37 | 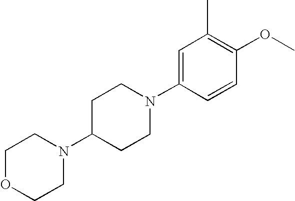 | 634.5 [M + 1]⁺ | CDCl₃: 1.57 (m, 2 H), 1.80 (d, 2 H), 2.22 (m, 1 H), 2.49 (t, 2 H), 2.60 (br, 4 H), 2.66 (d, 3 H), 3.41 (d, 2 H), 3.77 (br, 4 H), 3.85 (s, 3 H), 6.54 (dd, 1 H), 6.79 (d, 1 H), 7.22 (t, 1 H), 7.54 (s, 1 H), 7.59 (t, 1 H), 7.96 (m, 2 H), 8.28 (s, 1 H), 8.42 (d, 1 H), 9.00 (s, 1 H), |
| 38 | 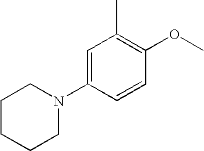 | Rf = 0.5 (hexane/ AcOEt = 1/1) | CDCl₃: 1.48 (m, 2 H), 1.57 (m, 4 H), 2.64 (d, 3 h), 2.85 (br, 4 H), 3.85 (s, 3 H), 4.53 (br, 1 H), 6.55 (d, 1 H), 6.79 (d, 1 H), 7.23 (t, 1 H), 7.55 (s, 1 H), 7.58 (t, 1 H), 7.95 (m, 2 H), 8.27 (s, 1 H), 8.43 (d, 1 H), 8.93 (s, 1 H). |
| 39 | 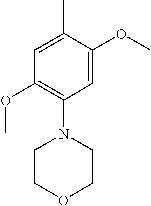 | 579 581 [M + 1]⁺ | DMSO-d₆: 2.43 (s, 3 H), 2.96-3.00 (m, 4 H), 3.55 (s, 3 H), 3.72-3.75 (m, 7 H), 6.62 (s, 1 H), 7.21-7.27 (m, 2 H), 7.41-7.45 (m, 1 H), 7.75-7.77 (m, 2 H), 8.26 (brs, 2 H), 8.37-8.40 (m, 1 H), 9.18 (s, 1 H) |
| 40 | 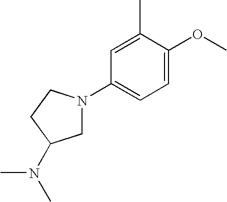 | 0.30 (CH₂Cl₂:MeOH) = 8:2 | DMSO-d₆: 1.65-1.75 (m, 1 H), 1.97-2.05 (m, 1 H), 2.14 (s, 6 H), 2.43 (s, 3 H), 2.67-2.75 (m, 1 H), 2.86-3.07 (m, 3 H), 3.22-3.27 (m, 1 H), 3.71 (s, 3 H), 6.25 (dd, 1 H), 6.91 (d, 1 H), 7.02 (brs, 1 H), 7.20-7.24 (m, 1 H), 7.40-7.44 (m, 1 H), 7.76-7.78 (m, 2 H), 8.19 (brs, 1 H), 8.30 (s, 1 H), 8.39-8.40 (m, 1 H), 9.18 (s, 1 H) |
| 41 | 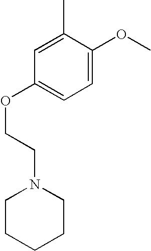 | 592 [M + 1]⁺ | DMSO-d₆: 1.34-1.40 (m, 2 H), 1.45-1.51 (m, 4 H), 2.36-2.42 (br, 4 H), 2.44 (s, 3 H), 2.58-2.60 (m, 2 H), 3.77 (s, 3 H), 3.87-3.90 (m, 2 H), 6.59 (dd, 1 H), 6.92 (d, 1 H), 7.24-7.28 (m, 2 H), 7.55-7.62 (m, 2 H), 8.10 (s, 1 H), 8.34 (s, 1 H), 8.38-8.41 (m, 1 H), 9.20 (s, 1 H) |
| 42 | 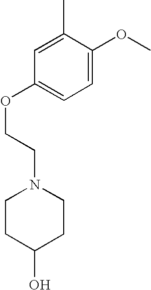 | 608 [M + 1]⁺ | DMSO-d₆: 1.33-1.41 (m, 2 H), 1.67-1.71 (m, 2 H), 2.06-2.11 (m, 2 H), 2.44 (s, 3 H), 2.59-2.62 (m, 1 H), 2.73-2.76 (m, 1 H), 3.39-3.46 (m, 1 H), 3.77 (s, 3 H), 3.85-3.90 (m, 2 H), 4.52-4.53 (m, 1 H), 6.58-6.61 (m, 1 H), 6.92-6.94 (m, 1 H), 7.24-7.28 (m, 1 H), 7.55-7.62 (m, 2 H), 7.75-7.81 (m, 2 H), 8.09 (s, 1 H), 8.34 (s, 1 H), 8.38-8.40 (m, 1 H), 9.19 (s, 1 H) |

### Example 43

The following 2-[5-bromo-2-(subst. phenylamino)-pyrimidin-4-ylamino]-N,N-dimethyl-benzene-sulfonamides are prepared from 2-(2-bromo-5-chloro-pyrimidin-4-ylamino)-N, N-dimethyl-benzenesulfonamide and the corresponding aniline following the procedure of Example 1 or Example 20:

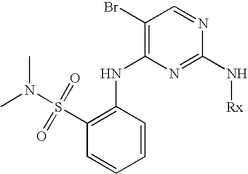

| Expl | | Mass (m/z) or | |
|---|---|---|---|
| No. | Rx | Rf (solvent) | NMR (400 MHz), δ (ppm) |
| 44 | 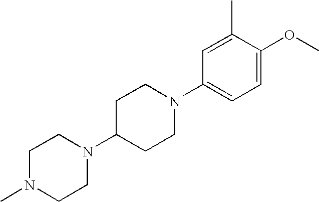 | 659, 661 [M + 1]⁺ | DMSO-d₆: 1.38-1.47 (m, 2 H), 1.70-1.79 (m, 2 H), 2.13 (s, 3 H), 2.15-2.48 (m, 11 H), 2.65 (s, 6 H), 3.30-3.46 (m, 3 H), 3.73 (s, 3 H), 6.63 (dd, 1 H), 6.90 (d, 1 H), 7.28-7.32 (m, 1 H), 7.36-7.42 (m, 1 H), 7.54-7.57 (m, 1 H), 7.77 (dd, 1 H), 8.21 (s, 1 H), 8.32 (s, 1 H), 8.38-8.44 (m, 1 H), 9.21 (s, 1 H) |
| 45 | 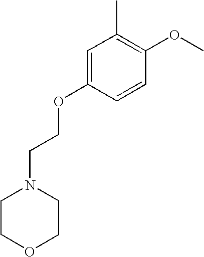 | Rf = 0.6 (CH₂Cl₂/ MeOH = 10/1) | CDCl₃: 2.56 (br, 4 H), 2.76 (s, 6 H), 2.78 (t, 2 H), 3.73 (t, 4 H), 3.86 (s, 3 H), 3.99 (t, 2 H), 6.47 (dd, 1 H), 6.77 (d, 1 H), 7.21 (t, 1 H), 7.60 (s, 1 H), 7.67 (t, 1 H), 7.88 (dd, 1 H), 8.02 (d, 1 H), 8.25 (s, 1 H), 8.50 (d, 1 H), 9.30 (s, 1 H). |
| 46 | 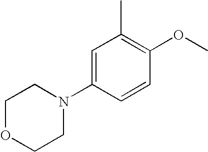 | 565.3 [M + 1]⁺ | CDCl₃: 2.73 (s, 6 H), 2.90 (br, 4 H), 3.74 (br, 4 H), 3.87 (s, 3 H), 6.52 (d, 1 H), 6.81 (d, 1 H), 7.22 (t, 1 H), 7.62 (t, 1 H), 7.87 (dd, 1 H), 8.00 (d, 1 H), 8.27 (s, 1 H), 8.44 (d, 1 H), 9.27 (s, 1 H). |

### Example 47

The following 2-[5-bromo-2-(subst. phenylamino)-pyrimidin-4-ylamino]-N-isopropyl-benzene-sulfonamides are prepared from 2-(2-bromo-5-chloro-pyrimidin-4-ylamino)-N-isopropyl-benzenesulfonamide and the corresponding aniline following the procedure of Example 1 or Example 20:

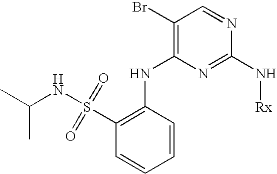

| Expl | | Mass (m/z) or | |
|---|---|---|---|
| No. | Rx | Rf (solvent) | NMR (400 MHz), δ (ppm) |
| 48 | 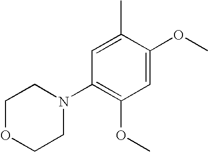 | 608, 610 [M + 1]⁺ | DMSO-d₆: 0.95 (d, 6 H), 2.68-2.73 (m, 4 H), 3.58-3.64 (m, 4 H), 3.77 (s, 3 H), 3.83 (s, 3 H), 6.73 (s, 1 H), 7.08-7.13 (m, 1 H), 7.16-7.21 (m, 1 H), 7.34-7.43 (m, 1 H), 7.79-7.81 (m, 1 H), 7.87-7.95 (m, 1 H), 8.20 (s, 1 H), 8.25-8.38 (m, 2 H), 9.15 (s, 1 H) |
| 49 | 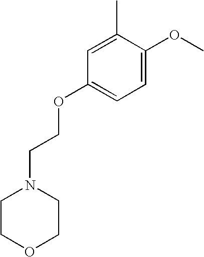 | 621 623 [M + 1]⁺ | CDCl3: 1.02 (d, 6 H), 2.53-2.55 (m, 4 H), 2.72 (t, 2 H), 3.41-3.50 (m, 1 H), 3.71-3.74 (m, 4 H), 3.85 (s, 3 H), 3.92 (t, 2 H), 4.38 (d, 1 H), 6.47 (dd, 1 H), 6.76.(d, 1 H), 7.22 (t, 1 H), 7.62 (s, 1 H), 7.67 (t, 1 H), 7.97-8.01 (m, 2 H), 8.27 (s, 1 H), 8.41 (d, 1 H), 8.83 (s, 1 H) |
| 50 | 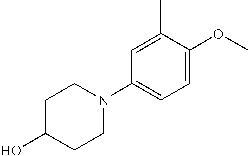 | 591 593 [M + 1]⁺ | DMSO-d₆: 0.95 (d, 6 H), 1.31-1.44 (m, 2 H), 1.63-1.75 1.75 (m, 2 H), 3.09-3.20 (m, 2 H), 3.42-3.53 (m, 1 H), 3.74 (s, 3 H), 6.59 (d, 1 H), 6.89 (d, 1 H), 7.23 (t. 1 H), 7.42-7.53 (m, 2 H), 7.77-7.94 (m, 2 H), 8.01 (s, 1 H), 8.26-8.35 (m, 2 H), 9.11 (s, 1 H) |
| 51 | 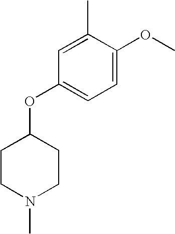 | 605 607 [M + 1] | DMSO-d₆: 0.95 (d, 3 H), 1.43-1.57 (m, 2 H), 1.73-1.84 (m, 2 H), 2.07 (t, 2 H), 2.33 (s, 3 H), 3.77 (s, 3 H), 4.01-4.05 (m, 1 H), 6.58 (d, 1 H), 6.91 (d, 1 H), 7.26 (t, 1 H), 7.52-7.57 (m, 2 H), 7.83-7.89 (m, 2 H), 8.04 (s, 1 H), 8.32-8.35 (m, 2 H), 9.13 (s, 1 H) |
| 52 | 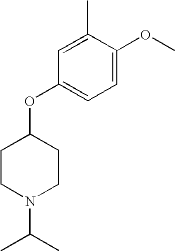 | 633 635 [M + 1] | DMSO-d₆: 0.92-0.96 (m, 12 H), 1.43-1.57 (m, 2 H), 1.76-1.86 (m, 2 H), 2.23 (t, 2 H), 2.65 (brs, 3 H), 3.77 (s, 3 H), 3.95-4.05 (m, 1 H), 6.57 (d, 1 H), 6.90 (d, 1 H), 7.26 (t, 1 H), 7.50-7.57 (m, 2 H), 7.80-7.89 (m, 2 H), 8.04 (s, 1 H), 8.28-8.36 (m, 2 H), 9.12 (s, 1 H) |
| 53 | 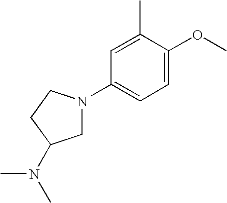 | 604 606 [M + 1] | DMSO-d₆: 0.96 (d, 6 H), 1.64-1.73 (m, 1 H), 1.94-2.03 (m, 1 H), 2.14 (s, 6 H), 2.67-2.75 (m, 1 H), 2.85-2.03 (m, 3 H), 3.23 (t, 1 H), 3.71 (s, 3 H), 6.23 (dd, 1 H), 6.89 (d, 1 H), 7.05 (s, 3 H), 7.22 (t, 1 H), 7.41 (t, 1 H), 7.82 (d, 1 H), 7.89 (d, 1 H), 8.13 (s, 1 H), 8.32 (s, 1 H), 8.36 (d, 1 H), 9.17 (s, 1 H) |
| 54 | 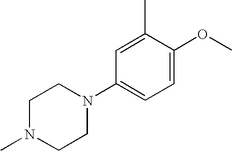 | 590 592 [M + 1] | DMSO-D6: 0.95 (d, 6 H), 2.18 (s, 3 H), 2.28-2.35 (m, 4 H), 2.78-2.85 (m, 4 H), 3.75 (s, 3 H), 6.58 (d, 1 H), 6.90 (d, 1 H), 7.23 (t, 1 H), 7.43-7.52 (m, 2 H), 7.84 (d, 1 H), 7.90 (d, 1 H), 5.09 (s, 1 H), 8.27-8.36 (m, 2 H), 9.12 (s, 1 H). |
| 55 | 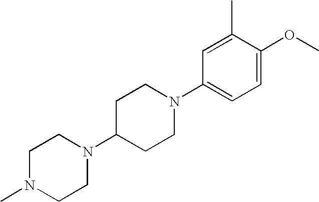 | 673, 675 [M + 1]⁺ | CDCl₃: 1.04 (d, 6 H), 1.48-1.66 (m, 3 H), 1.76 (d, 2 H), 2.18-2.30 (m, 1 H), 2.31 (s, 3 H), 2.48-2.67 (m, 9 H), 3.37 (d, 2 H), 3.42-3.47 (m, 1 H), 3.85 (s, 3 H), 4.44 (d, 1 H), 6.66 (dd, 1 H), 6.78 (d, 1 H), 7.22 (t, 1 H), 7.55-7.60 (m, 2 H), 7.93 (s, 1 H), 7.99 (d, 1 H), 8.28 (s, 1 H), 8.39 (d, 1 H), 8.86 (s, 1 H) |
| 56 | 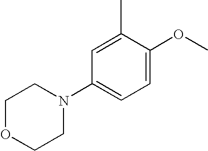 | 577, 579 [M + 1]⁺ | DMSO-d₆: 0.91 (d, 6 H), 2.70-2.75 (m, 4 H), 3.25-3.33 (m, 1 H), 3.53-3.58 (m, 4 H), 3.76 (s, 3 H), 6.55 (dd, 1 H), 6.88 (d, 1 H), 7.22 (t, 1 H), 7.43-7.49 (m, 2 H), 7.80 (d, 1 H), 7.87 (d, 1 H), 8.05 (s, 1 H), 8.24 (d, 1 H), 8.31 (s, 1 H), 9.06 (s, 1 H) |
| 57 | 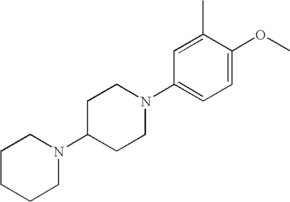 | 658 660 [M + 1]⁺ | DMSO-d₆: 0.94 (s, 6 H), 1.33-1.52 (m, 8 H), 1.60-1.68 (m, 2 H), 2.14-2.20 (m, 1 H), 2.32-2.45 (m, 9 H), 3.25-3.33 (m, 1 H), 3.74 (s, 3 H), 6.58 (d, 1 H), 6.88 (d, 1 H), 7.22 (t, 1 H), 7.43-7.51 (m, 2 H), 7.82 (d, 1 H), 7.90 (d, 1 H), 8.06 (s, 1 H), 8.27-8.35 (m, 2 H), 9.11 (s, 1 H) |
| 58 | 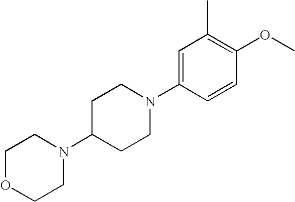 | Rf = 0.5 (CH₂Cl₂/ MeOH = 10/1) | CDCl₃: 1.10 (d, 6 H), 1.62 (m, 4 H), 1.85 (m, 2 H), 2.44 (t, 2 H), 2.73 (m, 4 H), 3.36 (d, 2 H), 3.35 (m, 1 H), 3.85 (s, 3 H) , 3.90 (m, 3 H), 6.52 (d, 1 H), 6.75 (d, 1 H), 7.25 (t, 1 H), 7.55 (m, 2 H), 7.97 (s, 1 H), 8.03 (d, 1 H), 8.27 (s, 1 H), 8.28 (m, 1 H), 8.86 (s, 1 H) |
| 59 | 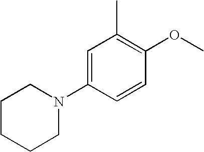 | Rf = 0.6 (hexane/ AcOEt = 1/1) | CDCl₃: 1.04 (d, 6 H), 1.57 (m, 6 H), 2.8 (br, 4 H), 3.46 (m, 1 H), 3.85 (s, 3 H), 4.41 (br, 1 H), 6.55 (br, 1 H), 6.79 (d, 1 H), 7.23 (t, 1 H), 7.58 (m, 2 H), 7.92 (s, 1 H), 7.99 (d, 1 H), 8.28 (s, 1 H), 8.39 (m, 1 H), 8.84 (s, 1 H) |

### Example 60

The following 2-[5-chloro-2-(2-methoxy-4-morpholino-phenylamino)-pyrimidin-4-ylamino]-N-substituted alkyl or N, N-dialkyl-benzenesulfonamides are prepared from 2-(2,5-dichloro-pyrimidin-4-ylamino)-N-substituted alkyl or N, N-dialkyl-benzenesulfonamide and 2-methoxy-4-morpholin-4-yl-phenylamine following the procedure of Example 1 or Example 20:

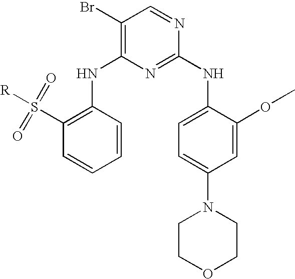

| Expl | | Mass (ESI) | NMR (400 MHz) δ (ppm) or |
|---|---|---|---|
| No. | Rx | m/z or Rf | HPLC Retention time (min) |
| 61 | 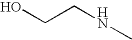 | 536 | CDCl3: 3.14-3.07 (m, 6 H), 3.49 (t, 2 H), 3.93-3.88 (m, 4 H), 3.88 (s, 3 H), 5.07 (t, 1 H), 6.46-6.40 (m, 1 H), 6.55 (s, 1 H), 7.30-7.23 (m, 1 H). 7.48-7.37 (m, 1 H), 7.62-7.58 (m, 1 H), 7.98 (dd, 1 H), 8.12 (s, 1 H), 8.36 (d, 1 H), 8.87 (s, 1 H) |
| 62 | 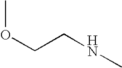 | 549 | CDCl3: 3.12 (s, 3 H), 3.27-3.09 (m, 6 H), 3.88 (t, 2 H), 3.89-3.87 (m, 4 H), 3.88 (s, 3 H), 5.00 (t, 1 H), 6.43 (dd, 1 H), 6.53 (d, 1 H), 7.29-7.22 (m, 1 H), 7.32 (s, 1 H), 7.61-7.56 (m, 1 H), 7.96 (dd, 1 H), 8.05 (d, 1 H), 8.13 (s, 1 H), 8.49 (d, 1 H), 8.98 (s, 1 H) |
| 63 | 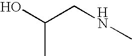 | 550 | CDCl3: 1.00 (d, 3 H), 1.74-1.73 (m, 1 H), 2.75 (ddd, 2 H), 3.05 (ddd, 2 H), 3.12-3.10 (m, 4 H), 3.75-3.64 (m, 1 H). 3.88-3.86 (m, 4 H), 3.87 (s, 3 H), 5.03-5.00 (m, 1 H), 6.40 (dd, 1 H), 6.52 (d, 1 H), 7.29-7.22 (m, 1 H), 7.32-7.29 (m, 1 H), 7.61-7.56 (m, 1 H), 7.62-7.58 (m, 1 H), 8.00-7.96 (m, 1 H), 8.13 (s, 1 H), 8.37 (d, 1 H), 8.82 (s, 1 H) |
| 64 | 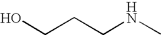 | 550 | CDCl3: 1.48-1.45 (m, 1 H), 1.61-1.51 (m, 2 H), 3.13-3.07 (m, 6 H), 3.55 (dd, 2 H), 3.89-3.86 (m, 4 H), 3.88 (s, 3 H), 5.34-5.30 (m, 1 H), 6.40 (dd, 1 H), 7.52 (d, 1 H), 7.32 (d, 1 H), 7.62-7.57 (m, 1 H), 7.98 (dd, 1 H), 8.02 (d, 1 H), 8.12 (s, 1 H), 8.39 (d, 1 H), 8.90 (s, 1 H) |
| 65 | 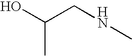 | 549 | CDCl3: 1.00 (d, 3 H), 1.74-1.73 (m, 1 H), 2.75 (ddd, 2 H), 3.05 (ddd, 2 H), 3.12-3.10 (m, 4 H), 3.75-3.64 (m, 1 H), 3.88-3.86 (m, 4 H), 3.87 (s, 3 H), 5.03-5.00 (m, 1 H), 6.40 (dd, 1 H), 6.52 (d, 1 H), 7.29-7.22 (m, 1 H), 7.32-7.29 (m, 1 H), 7.61-7.56 (m, 1 H), 7.62-7.58 (m, 1 H), 8.00-7.96 (m, 1 H), 8.13 (s, 1 H), 8.37 (d, 1 H), 8.82 (s, 1 H) |
| 66 | 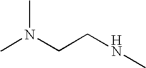 | MS 562, 564 | CDCl3: 2.01 (s, 6 H), 2.31 (t, 2 H), 3.00 (t, 1 H), 3.21-3.18 (m, 4H), 3.95 (s, 3 H), 3.97-3.94 (m, 4 H), 6.50 (dd, 1 H), 6.60 (d, 1 H), 7.31-7.29 (m, 1 H), 7.39 (s, 1 H), 7.68-7.63 (m, 1 H), 8.05 (dd, 1 H), 8.11 (d, 1 H), 8.21 (s, 1 H), 8.55 (d, 1 H), 9.09 (s, 1 H) |
| 67 | 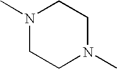 | MS 574, 576 | CDCl3: 2.28 (s, 3 H), 2.46 (t, 4 H), 3.22-3.20 (m, 8 H), 3.96 (s, 3 H), 3.97-3.95 (m, 4 H), 6.53 (dd, 1 H), 6.61 (d, 1 H), 7.31-7.27 (m, 1 H), 7.38 (s, 1 H), 7.65-7.61 (m, 1 H), 7.91 (dd, 1 H), 8.12 (d, 1 H), 8.19 (s, 1 H), 8.64 (dd, 1 H), 9.40 (s, 1 H) |
| 68 | 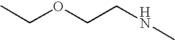 | MS 563 | CDCl3: 0.98 (t, 3 H), 3.14-3.09 (m, 6 H), 3.31-3.24 (m, 4 H), 3.88 (s, 3 H), 5.05 (m, 1 H), 6.43 (dd, 1 H), 6.53 (d, 1 H), 7.24-7.21 (m, 1 H), 7.31 (s, 1 H), 7.60-7.56 (m, 1 H), 7.38 (s, 1 H), 7.97 (dd, 1 H), 8.05 (dd, 1 H), 8.12 (s, 1 H), 8.13 (s, 1 H), 8.50 (d, 1 H), 9.00 (s, 1 H) |
| 69 | 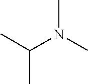 | MS: 547 | DMSO-d6: 0.9 (d, 6 H), 2.61 (s, 3 H), 3.1-3.13 (m, 4 H), 3.74-3.78 (m, 4 H), 3.75 (s, 3 H), 6.45 (dd, 1 H), 6.64 (d, 1 H), 7.23-7.29 (m, 1 H), 7.37-7.42 (m, 1 H), 7.47-7.54 (m, 1 H), 7.82 (m, 1 H), 8.18 (s, 1 H), 8.24 (s, 1 H) 8.44-8.52 (m, 1 H), 9.19 (s, 1 H) |
| 70 | 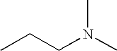 | MS: 547 | DMSO-d6: 0.72 (t, 3 H), 1.37-1.45 (m, 2 H), 2.68 (s, 3 H), 2.97 (t, 2 H), 2.95-2.99 (m, 4 H), 3.74-3.77 (m, 4 H), 3.76 (s, 3 H), 6.46 (dd, 1 H), 6.65 (d, 1 H), 7.24-7.29 (m, 1 H), 7.37-7.4 (m, 1 H), 7.5-7.54 (m, 1 H), 7.76-7.79 (m, 1 H), 8.18 (s, 1 H), 8.25 (s, 1 H) 8.49-8.51 (m, 1 H), 9.32 (s, 1 H) |
| 71 | 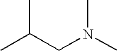 | R_{f} (hexane/Et OAc 1:2) 0.59. | CDCl3: 0.85 (d, 6 H), 1.82 (dq, 1 H), 2.73 (s, 3 H), 2.79 (d, 2 H), 3.08-3.16 (m, 4 H), 3.89 (s, 3 H), 3.85-3.92 (m, 4 H), 6.45 (dd, 1 H), 6.54 (d, 1 H), 7.22 (dd, 1 H), 7.31 (br. S, 1 H), 7.56 (dd, 1 H), 7.87 (d, 1 H), 8.07 (d, 1 H), 8.11 (s, 1 H), 8.51 (d, 1 H), 9.37 (br. S, 1 H). |
| 72 | 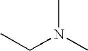 | R_{f} (hexane/Et OAc 2:5) 0.63. | CDCl3: 1.07 (t, 3 H), 2.75 (s, 3 H), 3.11-3.17 (m, 4 H), 3.19 (q, 2 H), 3.89 (s, 3 H), 3.85-3.91 (m, 4 H), 6.46 (dd, 1 H), 6.53 (d, 1 H), 7.20 (dd, 1 H), 7.31 (br. s, 1 H), 7.57 (dd, 1 H), 7.89 (d, 1 H), 8.08 (d, 1 H), 8.12 (s, 1 H), 8.53 (d, 1 H), 9.30 (br. s, 1 H). |

### Example 73

### Preparation of 5-chloro-N²-(2-methoxy-4-morpholin-4-yl-phenyl)-N⁴-[2-(piperazine-1-sulfonyl)-phenyl]-pyrimidine-2,4-diamine

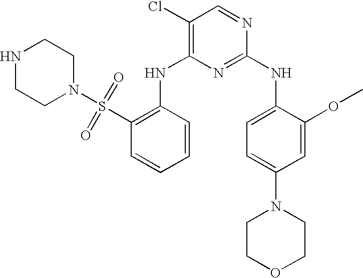

Deprotection by using hydrogen bromide in acetic acid of 5-chloro-N²-(2-methoxy-4-morpholin-4-yl-phenyl)-N⁴-[2-(4-benzyloxycarbonyl-piperazine-1-sulfonyl)-phenyl]-pyrimidine-2,4-diamine obtained following the procedure of Example 1 affords 5-chloro-N²-(2-methoxy-4-morpholin-4-yl-phenyl)-N⁴-[2-(piperazine-1-sulfonyl)-phenyl]-pyrimidine-2,4-diamine. ESI-MS (m/z): 560 [MH]⁺, ¹H-NMR (400 MHz, δ, ppm) CDCl3: 2.86-2.83 (m, 4H), 3.07-3.05 (m, 4H), 3.15-3.12 (m, 4H), 3.89 (s, 3H), 3.90-3.88 (m, 4H), 6.47 (dd, 1H), 6.54 (d, 1H), 7.27-7.20 (m, 1H), 7.30 (s, 1H), 7.59-7.52 (m, 1H), 7.84 (d, 1H), 8.06 (d, 1H), 8.12 (s, 1H), 8.58 (d, 1H), 9.34 (s, 1H).

### Example 74

The following 2-{5-chloro-2-[2-methoxy-4-(4-methyl-piperazin-1-yl)-phenylamino]-pyrimidin-4-ylamino}-N,N-alkyl-benzenesulfonamides are prepared from 2-(2,5-dichloro-pyrimidin-4-ylamino)-N,N-dialkylyl-benzenesulfonamide and 2-methoxy-4-(4-methyl-piperazin-1-yl)-phenylamine following the procedure of Example 1 or Example 20:

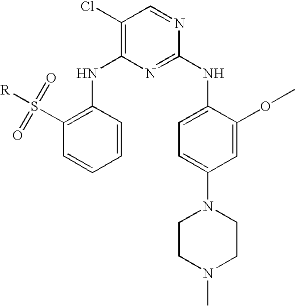

| Expl | | Mass (ESI) | NMR (400 MHz) δ (ppm) or |
|---|---|---|---|
| No. | Rx | m/z or Rf | HPLC Retention time (min) |
| 75 | 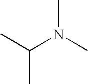 | R_{f} (CH₂Cl₂/ MeOH 10:1) 0.42. | CDCl3: 0.98 (d, 6 H), 2.58-2.64 (m, 4 H), 2.68 (s, 3 H), 2.69 (s, 3 H), 3.16-3.21 (m, 4H), 3.87 (s, 3 H), 4.20 (dq, 1 H), 6.42 (dd, 1 H), 6.54 (d, 1 H), 7.20 (dd, 1 H), 7.29 (br.s, 1 H), 7.53 (dd, 1 H), 7.91 (d, 1 H), 8.02 (d, 1 H), 8.11 (s, 1 H), 8.47 (d, 1 H), 9.17 (br.s, 1 H). |
| 76 | 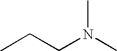 | R_{f} (CH₂Cl₂/ MeOH 10:1) 0.37. | CDCl3: 0.82 (t, 3 H), 1.45-1.54 (m, 2 H), 2.58-2.67 (m, 4 H), 2.68 (s, 1 H), 2.69 (s, 3 H), 2.97-3.02 (m, 2 H), 3.16-3.21 (m, 4 H), 3.86 (s, 3 H), 6.46 (dd, 1 H), 6.55 (d, 1 H), 7.20 (dd, 1 H), 7.29 (br.s, 1 H), 7.52 (dd, 1 H), 7.88 (d, 1 H), 8.04 (d, 1 H), 8.10 (s, 1 H), 8.50 (d, 1 H), 9.30 (br.s, 1 H). |
| 77 | 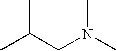 | R_{f} (CH₂Cl₂/ EtOH 6:1) 0.44. | CDCl3: 0.86 (d, 6 H), 1.82 (dq, 1 H), 2.38 (s, 3 H), 2.58- 2.64 (m, 4 H), 2.71 (s, 3 H), 2.80 (d, 2H), 3.13-3.21 (m, 4 H), 3.88 (s, 3 H), 6.45 (dd, 1 H), 6.54 (d, 1 H), 7.21 (dd, 1 H), 7.31 (br.s, 1 H), 7.54 (dd, 1 H), 7.88 (d, 1 H), 8.05 (d, 1 H), 8.11 (s, 1 H), 8.53 (d, 1 H), 9.34 (br.s, 1 H). |
| 78 | 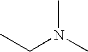 | R_{f} (CH₂Cl₂/ EtOH 4:1) 0.60. | CDCl3: 1.07 (t, 3 H), 2.38 (s, 3 H), 2.57-2.62 (m, 4 H), 2.75 (s, 3 H), 3.11-3.20 (m, 7 H), 3.89 (s, 3 H), 6.45 (dd, 1 H), 6.55 (d, 1 H), 7.20 (dd, 1 H), 7.30 (br.s, 1 H), 7.55 (dd, 1 H), 7.89 (d, 1 H), 8.05 (d, 1 H), 8.11 (s, 1 H), 8.52 (d, 1 H), 9.30 (br.s, 1 H). |

### Example 79

The following 2-[5-Chloro-2-(substituted phenylamino)-pyrimidin-4-ylamino]-N-methyl-5-(4-methyl-piperazine-i-yl)-benzamide are prepared from 2-(2,5-Dichloro-pyrimidin-4-ylamino)-N-methyl-5-(4-methyl-piperazin-1-yl)-benzamide and the corresponding aniline following the procedure of Example 20.

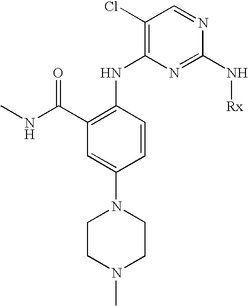

| Expl | | Rf (solvent) | |
|---|---|---|---|
| No. | Rx | Or MS (ESI) | NMR (400 MHz), δ (ppm) |
| 80 | 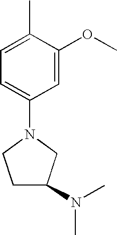 | 594, 596 | DMSO-d6: 1.75-1.86 (m, 1 H), 2.13-2.2 (m, 1 H), 2.22 (s, 6 H), 2.24 (s, 3 H), 2.44-2.5 (m, 4H), 2.75-2.84 (m, 1 H), 2.78 (d, 3 H), 3.03-3.15 (m, 5H), 3.36-3.43 (m, 2 H), 3.46-3.52 (m, 1 H), 3.74 (s, 3 H), 6.11 (dd, 1 H), 6.23 (d, 1 H), 6.72-6.84 (m, 1 H), 7.18 (d, 1 H), 7.22 (d, 1 H), 7.98 (s, 1 H), 7.99 (s, 1 H), 8.25-8.36 (m, 1 H), 8.62-8.67 (m, 1 H), 11.12 (s, 1 H). |
| 81 | 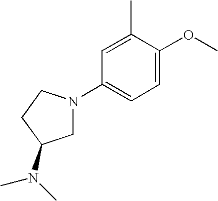 | 594, 596 | DMSO-d6: 1.65-1.78 (m, 1 H), 2.01-2.10 (m, 1 H), 2.14 (s, 6 H), 2.24 (s, 3 H), 2.44-2.5 (m, 5 H), 2.65-2.76 (m, 1 H), 2.79 (d, 3 H), 2.91 (dd, 1 H), 3.02-3.11 (m, 1 H), 3.12-3.17 (m, 4H), 3.19-3.26 (m, 1 H), 3.72 (s, 3 H), 6.24 (dd, 1 H), 6.85-6.92 (m, 2 H), 7.13 (br.s, 1 H), 7.21 (d, 1 H), 7.93 (s, 1 H), 8.11 (s, 1 H), 8.41 (d, 1 H), 8.66- 8.72 (m, 1 H), 11.28 (s, 1 H). |
| 82 | 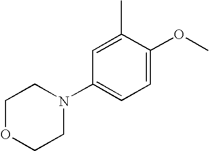 | 567, 569 | DMSO-d6: 2.24 (s, 3 H), 2.44-2.50 (m, 4 H), 2.75-2.82 (d, 3 H), 2.84-2.91 (m, 4 H), 3.12-3.20 (m, 4 H), 3.77 (s, 3 H), 6.60 (dd, 1 H), 6.93 (d, 1 H), 6.95-7.02 (m, 1 H), 7.18-7.23 (m, 1 H), 7.55-7.62 (m, 1 H), 7.92 s, 1 H), 8.13 (s, 1 H), 8.35 (d, 1 H), 8.66-8.73 (m, 1 H), 11.21 (s, 1 H). |

### Example 83

The following 2-[5-Chloro-2-(substituted phenylamino)-pyrimidin-4-ylamino]-5-(3-(S)-dimethylamino-pyrrolidin-1-yl)-N-methyl-benzamide are prepared from 2-(2,5-Dichloro-pyrimidin-4-ylamino)-5-(3-(S)-dimethylamino-pyrrolidin-1-yl)-N-methyl-benzamide and the corresponding aniline following the procedure of Example 20.

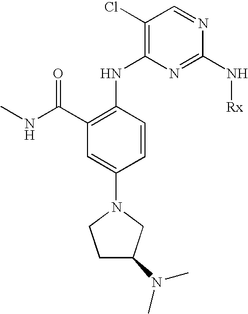

| Expl | | Rf (solvent) | |
|---|---|---|---|
| No. | Rx | Or MS (ESI) | NMR (400 MHz), δ (ppm) |
| 84 | 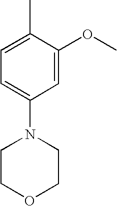 | 581, 583 | DMSO-d6: 1.75-1.88 (m, 1 H), 2.12-2.2 (m, 1 H), 2.22 (s, 6 H), 2.78 (d, 3 H), 2.8-2.85 (m, 1 H), 3.06 (dd, 1 H), 3.08-3.17 (m, 4 H), 3.21-3.3 (m, 1 H), 3.35-3.42 (m, 1 H), 3.43-3.50 (m, 1 H), 3.70-3.80 (m, 7 H), 6.45-6.53 (m, 2 H), 6.65 (d, 1 H), 6.78 (d, 1 H), 7.46 (d, 1 H), 7.91 (s, 1 H), 7.99 (s, 1 H), 8.23 (d, 1 H), 8.56-8.63 (m, 1 H), 10.91 (s, 1 H). |

### Example 85

The following 5-[1,4′]Bipiperidinyl-1′-yl-2-[5-Chloro-2-(substituted phenylamino)-pyrimidin-4-ylamino]-N-methyl-benzamide are prepared from 5-[1,4′]Bipiperidinyl-1′-yl-2-(2,5-dichloro-pyrimidin-4-ylamino)-N-methyl-benzamide and the corresponding aniline following the procedure of Example 20.

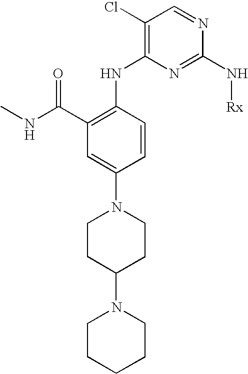

| | | Mass (m/z) | |
|---|---|---|---|
| Expl | | or Rf | |
| No. | Rx | (solvent) | NMR (400 MHz) δ (ppm) |
| 86 | 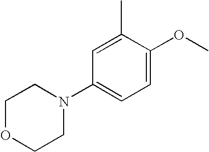 | 635, 637 | DMSO-d6: 1.36-1.42 (m, 2 H), 1.44-1.60 (m, 7 H), 1.76- 1.86 (m, 2 H), 2.27-2.38 (m, 1 H), 2.43-2.5 (m, 3 H), 2.59-2.69 (m, 2 H), 2.78 (d, 3 H), 2.85-2.92 (m, 4 H), 3.62-3.69 (m, 4 H), 3.71-3.80 (m, 5 H), 6.60 (dd, 1 H), 6.93 (d, 1 H), 6.98 (dd, 1 H), 7.20 (d, 1 H), 7.55-7.60 (m, 1 H), 7.92 (s, 1 H), 8.12 (s, 1 H), 8.34 (d, 1 H), 8.67-8.76 (m, 1 H), 11.20 (s, 1 H). |
| 87 | 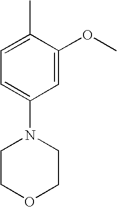 | 635, 637 | DMSO-d6: 1.33-1.66 (m, 10 H), 1.76-14.93 (m, 2 H), 2.50-2.70 (m, 5 H), 2.78 (d, 3 H), 3.08-3.15 (m, 4 H), 3.68-3.82 (m, 9 H), 6.49 (dd, 1 H), 6.66 (d, 1 H), 6.83- 6.91 (m, 1 H), 7.16-7.22 (m, 1 H), 7.41 (d, 1 H), 7.97- 8.14 (m, 2 H), 8.25-8.33 (m, 1 H), 8.62-8.70 (m, 1 H), 11.12 (s, 1 H). |

### Example 88

### Preparation of 2-[5-Chloro-2-(2-methoxy-4-morpholin-4-yl-phenylamino)-pyrimidin-4-ylamino]-5-(4-hydroxy-piperidin-1-yl)-N-methyl-benzamide

A suspension of acetic acid 1-[4-(2,5-dichloro-pyrimidin-4-ylamino)-3-methylcarbamoyl-phenyl]-piperidinyl-4-ester (200 mg, 0.456 mmol), 2-methoxy-4-morpholin-4-yl-phenylamine (128 mg, 0.455 mmol) and 1N hydro chloride in ethanol (0.46 mL) in 2-pentanol (5 mL) is stirred at 115° C. for 10 hours. To the mixture, water and sodium hydrogen carbonate aq are added and the mixture is extracted with ethyl acetate. The organic layer is washed with brine, dried over sodium sulfate and evaporated in vacuo. The residue is dissolved in methanol (5 mL), 3N sodium hydroxide is added to the solution and the mixture is stirred at room temperature for 30 min. The mixture is extracted with ethyl acetate. The organic layer is washed with brine, dried over sodium sulfate, evaporated in vacuo. The residue is purified by silica gel column chromatography (AcOEt; AcOEt: MeOH=20:1˜10:1). The resulting solids are dissolved in 1N hydrochloric acid and then neutralized with 1N sodium hydroxide. The precipitates are collected by filtration to give 2-[5-Chloro-2-(2-methoxy-4-morpholin-4-yl-phenylamino)-pyrimidin-4-ylamino]-5-(4-hydroxy-piperidin-1-yl)-N-methyl-benzamide (59 mg, 23%). ESI-MS (m/z): 568, 570 [MH]⁺, 1H-NMR (400 MHz, δ, ppm) DMSO-d6: 1.42-1.54 (m, 2H), 1.77-1.86 (m, 2H), 2.76 (d, 3H), 2.77-2.86 (m, 2H), 3.08-3.15 (m, 4H), 3.45-3.53 (m, 2H), 3.57-3.66 (m, 1H), 3.70-3.81 (m, 7H), 4.68 (brs, 1H), 6.44-6.49 (m, 1H), 6.65 (d, 1H), 6.80-6.88 (m, 1H), 7.17 (d, 1H), 7.37-7.41 (m, 1H), 7.98-8.02 (m, 2H), 8.21-8.28 (m, 1H), 8.60-8.66 (m, 1H), 11.09 (s, 1H).

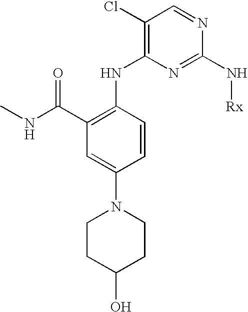

### Example 88-1

The following 2-[5-Chloro-2-(substituted phenylamino)-pyrimidin-4-ylamino]-5-(4-hydroxy-piperidin-1-yl)-N-methyl-benzamide are prepared from 2-(2,5-Dichloro-pyrimidin-4-ylamino)-5-(4-hydroxy-piperidin-1-yl)-N-methyl-benzamide and the corresponding aniline following the procedure of Example 88.

| | | Mass (m/z) | |
|---|---|---|---|
| Expl | | or Rf | |
| No. | Rx | (solvent) | NMR (400 MHz) δ (ppm) |
| 89 | 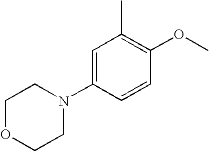 | 568, 570 | DMSO-d6: 1.44-1.56 (m, 2 H), 1.80-1.88 (m, 2 H), 2.79 (d, 3 H), 2.81-2.92 (m, 6 H), 3.50-3.58 (m, 2 H), 3.59- 3.69 (m, 6 H), 3.77 (s, 3 H), 6.61 (dd, 1 H), 6.93 (d, 1 H), 6.98 (dd, 1 H), 7.21 (d, 1 H), 7.58 (d, 1 H), 7.92 (s, 1 H), 8.12 (s, 1 H), 8.34 (d, 1 H), 8.67-8.72 (m, 1 H), 11.20 (s, 1 H). |

### Example 90

The following 7-[5-Chloro-2-(substituted phenylamino)-pyrimidin-4-ylamino]-2-methyl-2,3-dihydro-isoindol-1-ones are prepared from 7-(2,5-Dichloro-pyrimidin-4-ylamino)-2-methyl-2,3-dihydro-isoindol-1-one and the corresponding aniline following the procedure of Example 1 or Example 20.

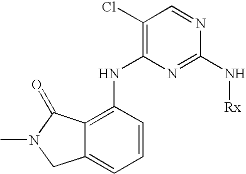

| | | Mass (m/z) | |
|---|---|---|---|
| Expl | | or Rf | |
| No. | Rx | (solvent) | NMR (400 MHz) δ (ppm) |
| 91 | 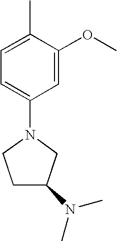 | 508, 510 | DMSO-d6: 1.78-1.91 (m, 1 H), 2.12-2.27 (m, 7 H), 2.77- 2.88 (m, 1 H), 3.03-3.13 (m, 4 H), 3.25-3.3 (m, 1 H), 3.36-3.45 (m, 1 H), 3.46-3.53 (m, 1 H), 3.73 (s, 3 H), 4.46 (s, 2 H), 6.15 (dd, 1 H), 6.24 (d, 1 H), 7.13 (d, 1 H), 7.2-7.3 (m, 1 H), 7.21 (d, 1 H), 8.09 (s, 1 H), 8.29 (s, 1 H), 8.22-8.58 (m, 1 H), 10.54 (s, 1 H). |
| 92 | 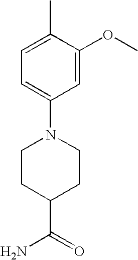 | 522, 524 | DMSO-d6: 1.61-1.76 (m, 2 H), 1.77-1.86 (m, 2 H), 2.21- 2.31 (m, 1 H), 2.64-2.75 (m, 2 H), 3.07 (s, 3 H), 3.74 (s, 3 H), 3.70-3.79 (m, 2 H), 4.46 (s, 2 H), 6.53 (dd, 1 H), 6.65 (d, 1 H), 6.78 (br.s, 1 H), 7.14 (d, 1 H), 7.25-7.36 (m, 3 H), 8.12 (s, 1 H), 8.34 (s, 1 H), 8.43 (br.s, 1 H), 10.57 (s, 1 H). |
| 93 | 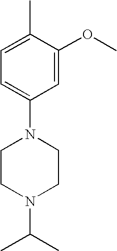 | 522, 524 | DMSO-d6: 1.02 (d, 6 H), 2.58-2.63 (m, 4 H), 2.64-2.73 (m, 1 H), 3.07 (s, 3 H), 3.12-3.18 (m, 4 H), 3.74 (s, 3 H), 4.46 (s, 2 H), 6.52 (dd, 1 H), 6.64 (d, 1 H), 7.15 (d, 1 H), 7.23-7.36 (m, 2 H), 8.12 (s, 1 H), 8.34 (s, 1 H), 8.37- 8.50 (m, 1 H), 10.57 (s, 1 H). |
| 94 | 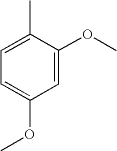 | 426, 428 | DMSO-d6: 3.07 (s, 3 H), 3.74 (s, 3 H), 3.81 (s, 3 H), 4.46 (s, 2 H), 6.56 (dd, 1 H), 6.67 (d, 1 H), 7.14 (d, 1 H), 7.29 (br.dd, 1 H), 7.39 (d, 1 H), 8.13 (s, 1 H), 8.35-8.44 (m, 1 H), 8.43 (s, 1 H), 10.57 (s, 1 H). |
| 95 | 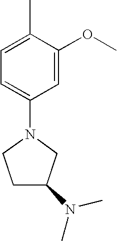 | 508, 510 | DMSO-d6: 1.79-1.91 (m, 1 H), 2.13-2.26 (m, 7 H), 2.78- 2.87 (m, 1 H), 3.04-3.13 (m, 4 H), 3.36-3.45 (m, 2 H), 3.46-3.52 (m, 1 H), 3.74 (s, 3 H), 4.46 (s, 2 H), 6.15 (dd, 1 H), 6.24 (d, 1 H), 7.13 (d, 1 H), 7.18-7.34 (m, 2 H), 8.09 (s, 1 H), 8.28 (s, 1 H), 8.32-8.54 (m, 1 H), 10.54 (s, 1 H). |
| 96 | 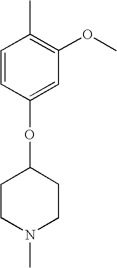 | Ms: 509 | 1.63-1.73 (m, 2 H), 1.92-2.0 (m, 2 H), 2.29-2.38 (m, 2 H), 2.63-2.7 (m, 2 H), 3.07 (s, 3 H), 3.73 (s, 3 H), 4.36- 4.46 (m, 1 H), 4.46 (s, 2 H), 6.58 (dd, 1 H), 6.67 (d, 1 H), 7.15 (d, 1 H), 7.24-7.33 (m, 1 H), 7.37 (d, 1 H), 8.13 (s, 1 H), 8.35-8.46 (m, 1 H), 8.42 (s, 1 H), 10.6 (s, 1 H) |
| 97 | 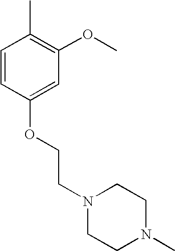 | Ms: 538 | 2.16 (s, 3 H), 2.2-2.4 (m, 4 H), 2.4-2.6 (m, 4 H), 2.71 (t, 2 H), 3.07 (s, 3 H), 3.74 (s, 3 H), 4.12 (t, 2 H), 4.46 (s, 2 H), 6.56 (dd, 1 H), 6.67 (d, 1 H), 7.14 (d, 1 H), 7.29 (t, 1 H), 7.38 (d, 1 H), 7.71 (dd, 1 H), 8.13 (s, 1 H), 8.3- 8.4 (m, 1 H), 8.42 (s, 1 H), 10.6 (s, 1 H) |
| 98 | 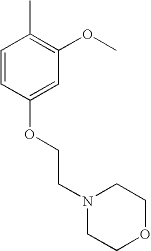 | Ms: 525 | 2.45-2.55 (m, 4 H), 2.72 (t, 2 H), 3.07 (s, 3 H), 3.6 (t, 4 H), 3.74 (s, 3 H), 4.14 (t, 2 H), 4.47 (s, 2 H), 6.57 (dd, 1 H), 6.68 (d, 1 H), 7.15 (d, 1 H), 7.3 (t, 1 H). 7.39 (d, 1 H), 8.13 (s, 1 H), 8.35-8.45 (m, 1 H), 8.42 (s, 1 H), 10.6 (s, 1 H) |
| 99 | 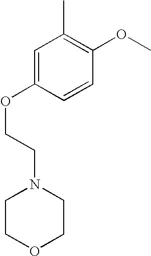 | Ms: 525 | 2.43 (t, 4 H), 2.65 (t, 2 H), 3.08 (s, 3 H), 3.56 (t, 4 H), 3.77 (s, 3 H), 4.0 (t, 2 H), 4.49 (s, 2 H), 6.69 (dd, 1 H), 6.98 (d, 1 H), 7.19 (d, 1 H), 7.43 (t, 1 H), 7.51 (d, 1 H), 8.25 (s, 1 H), 8.35 (s, 1 H), 8.55 (d, 1 H), 10.7 (s, 1 H) |
| 100 | 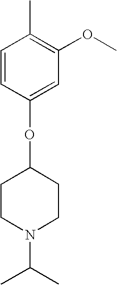 | Ms: 537 | 0.99 (d, 6 H), 1.57-1.67 (m, 2 H), 1.93-2.03 (m, 2 H), 2.29-2.38 (m, 2 H), 2.68-2.77 (m, 2 H), 3.07 (s, 3 H), 3.73 (s, 3 H), 4.33-4.41 (m, 1 H), 4.47 (s, 2 H), 6.57 (dd, 1 H), 6.66 (d, 1 H), 7.15 (d. 1 H), 7.25-7.32 (m, 1 H), 7.36 (d, 1 H), 8.13 (s, 1 H), 8.35-8.45 (m, 1 H), 8.41 (s, 1 H), 10.6 (s, 1 H) |
| 101 | 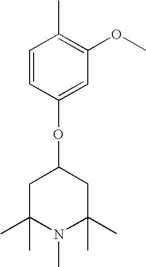 | Ms: 565 | 1.13 (s, 6 H), 1.21 (s, 6 H), 1.55-1.63 (m, 2 H), 2.03-2.07 (m, 2 H), 2.29 (s, 3 H), 3.2 (s, 3 H), 3.86 (s, 3 H), 4.38 (s, 2 H), 4.48-4.56 (m, 1 H), 6.55 (d, 1 H), 7.04 (d, 1 H), 7.12 (s, 1 H), 7.26 (s, 1 H), 7.44 (t, 1 H), 8.04 (d, 1 H), 8.11 (s, 1 H), 8.68 (d, 1 H), 10.6 (s, 1 H) |
| 102 | 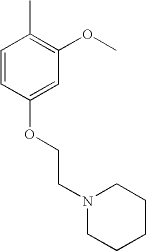 | Ms: 523 | 1.35-1.43 (m, 2 H), 1.47-1.55 (m, 4 H), 2.42-2.49 (m, 4 H), 2.68 (t, 2 H), 3.07 (s, 3 H), 3.74 (s, 3 H), 4.11 (t, 2 H), 4.47 (s, 2 H), 6.56 (dd, 1 H), 6.67 (d, 1 H), 7.14 (d, 1 H), 7.3-7.34 (m, 1 H), 7.37 (d, 1 H), 8.13 (s, 1 H), 8.33-8.45 (m, 1 H), 8.42 (s, 1 H), 10.6 (s, 1 H) |
| 103 | 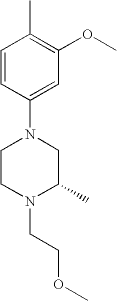 | Ms: 552 | 1.09 (d, 3 H), 2.39-2.59 (m, 3 H), 2.78-2.98 (m, 3 H), 3.07 (s, 3 H), 3.26 (s, 3 H), 3.3-3.4 (m, 1 H), 3.43-3.5 (m, 4 H), 3.74 (s, 3 H), 4.46 (s, 2 H), 6.51 (dd, 1 H), 6.64 (d, 1 H), 7.15 (d, 1 H), 7.25-7.35 (m, 2 H), 8.12 (s, 1 H), 8.34 (s, 1 H), 8.35-8.5 (m, 1 H), 10.6 (s, 1 H) |
| 104 | 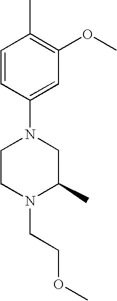 | Ms: 552 | 1.09 (d, 3 H), 2.4-2.6 (m, 3 H), 2.78-2.99 (m, 3 H), 3.07 (s, 3 H), 3.26 (s, 3 H), 3.3-3.4 (m, 1 H), 3.43-3.5 (m, 4 H), 3.74 (s, 3 H), 4.46 (s, 2 H), 6.51 (dd, 1 H), 6.64 (d, 1 H), 7.15 (d, 1 H), 7.25-7.35 (m, 2 H), 8.12 (s, 1 H), 8.34 (s, 1 H), 8.35-8.5 (m, 1 H), 10.6 (s, 1 H) |
| 105 | 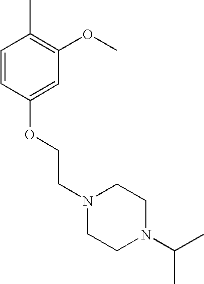 | Ms: 565 | 0.96 (d, 6 H), 2.4-2.55 (m, 8 H), 2.55-2.63 (m, 1 H), 2.7 (t, 2 H), 3.07 (s, 3 H), 3.74 (s, 3 H), 4.11 (t, 2 H), 4.47 (s, 2 H), 6.56 (dd, 1 H), 6.68 (d, 1 H), 7.14 (d, 1 H), 7.25- 7.35 (m, 1 H), 7.38 (d, 1 H), 8.13 (s, 1 H), 8.35-8.45 (m, 1 H), 8.42 (s, 1 H), 10.6 (s, 1 H) |
| 106 | 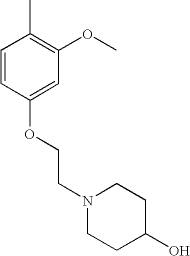 | Ms: 539 | 1.35-1.45 (m, 2 H), 1.68-1.77 (m, 2 H), 2.1-2.2 (m, 2 H), 2.68 (t, 3 H), 2.76-2.85 (m, 2 H), 3.07 (s, 3 H), 3.4-3.5 (m, 1 H), 3.74 (s, 3 H), 4.1 (t, 2 H), 4.47 (s, 2 H), 4.54 (d, 1 H), 6.56 (dd, 1 H), 6.68 (d, 1 H), 7.14 (d, 1 H), 7.25- 7.35 (m, 1 H), 7.38 (d, 1 H), 8.13 (s, 1 H), 8.35-8.45 (m, 1 H), 8.42 (s, 1 H), 10.6 (s, 1 H) |
| 107 | 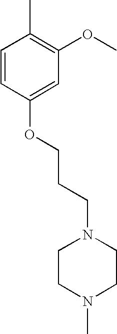 | Ms: 552 | 1.89 (t, 2 H), 2.15 (s, 3 H), 2.2-2.5 (m, 8 H), 2.44 (t, 2 H), 3.07 (s, 3 H), 3.74 (s, 3 H), 4.04 (t, 2 H), 4.47 (s, 2 H), 6.55 (dd, 1 H), 6.65 (d, 1 H), 7.14 (d, 1 H), 7.29 (t, 1 H), 7.38 (d, 1 H), 7.71 (dd, 1 H), 8.13 (s, 1 H), 8.3-8.4 (m, 1 H), 8.42 (s, 1 H), 10.6 (s, 1 H) |
| 108 | 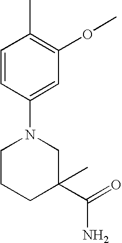 | MS 536, 538 | CDCl3: 1.25 (s, 3 H), 1.31 (dd, 1 H), 1.83-1.72 (m, 1 H), 1.99-1.85 (m, 1 H), 2.17-2.09 (m, 1 H), 2.71-2.59 (m, 2 H), 3.21 (s, 3 H), 3.53 (d, 1 H), 3.60 (d, 1 H), 3.91 (s, 3 H), 4.40 (s, 2 H), 5.54-5.42 (m, 1 H), 6.68-6.64 (m, 2 H), 7.07 (d, 1 H), 7.30-7.24 (m, 1 H), 7.46 (t, 1 H), 7.95-7.81 (m, 1 H), 8.12 (s, 1 H), 8.15 (d, 1 H), 8.70 (d, 1 H), 10.6 (s, 1 H). |
| 109 | 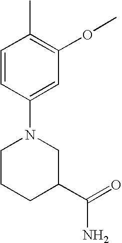 | MS 523 | CDCl3: 2.01-1.74 (m, 4 H), 2.72-2.65 (m, 1 H), 3.21 (s, 3 H), 3.27-3.08 (m, 2 H), 3.40-3.28 (m, 2 H), 3.90 (s, 1 H), 4.39 (s, 1 H), 5.50-5.38 (m, 1 H), 6.65-6.60 (m, 2 H), 6.83-6.74 (m, 1 H), 7.22 (s, 1 H), 7.51-7.44 (m, 1 H), 8.15-8.10 (m, 2 H), 8.70 (d, 1 H). |
| 110 | 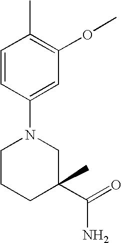 | MS 536, 538 | CDCl3: 1.25 (s, 3 H), 1.36-1.24 (m, 2 H), 1.97-1.90 (m, 1 H), 2.18-2.10 (m, 1 H), 2.62 (d, 1 H), 2.67 (dd, 1 H), 3.21 (s, 3 H), 3.57-3.50 (m, 1 H), 3.60 (d, 1 H), 3.92 (s, 3 H), 4.40 (s, 2 H), 5.51-5.44 (m, 1 H), 6.69-6.64 (m, 2 H), 7.07 (d, 1 H), 7.30-7.24 (m, 1 H), 7.38 (t, 1 H), 7.93-7.84 (m, 1 H), 8.12 (s, 1 H), 8.15 (d, 1 H), 8.70 (d, 1 H),10.6 (s, 1 H). |
| 111 | 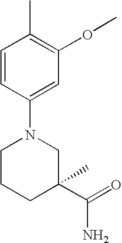 | 536, 538 | CDCl3: 1.25 (s, 3 H), 1.37-1.26 (m, 1 H), 1.82-1.73 (m, 1 H), 1.97-1.86 (m, 1 H), 2.15-2.07 (m, 1 H), 2.62 (d, 1 H), 2.69-2.61 (m, 1 H), 3.21 (s, 3 H), 3.56-3.49 (m, 1 H), 3.60 (d, 1 H), 3.91 (s, 3 H), 4.39 (s, 2 H), 5.50 (s, 1 H), 6.68-6.63 (m, 2 H), 7.07 (d, 1 H), 7.46 (t, 1 H), 7.86 (s, 1 H), 8.12 (s, 1 H), 8.15 (d, 1 H), 8.70 (d, 1 H), 10.6 (s, 1 H). |
| 112 | 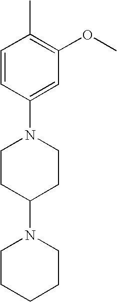 | 562, 564 | CDCl3: 1.42-1.50 m, 2 H). 1.58-1.67 (m, 4 H), 1.69-1.81 (m, 2 H), 1.89-1.97 (m, 2 H), 2.35-2.46 (m, 1 H), 2..35- 2.46 (m, 1 H), 2.53-2.59 (m, 4 H), 2.67-2.76 (m, 2 H), 3.20 (s, 3 H), 3.66-3.72 (m, 2 H), 3.88 (s, 3 H), 4.38 (s, 2 H), 6.56-6.60 (m, 2 H), 7.04 (d, 1 H), 7.15 (s, 1 H), 7.44 (dd, 1 H), 7.99-8.04 (m, 1 H), 8.10 (s, 1 H), 8.69 (d, 1 H), 10.58 (s, 1 H). |

### Example 113

The following 8-[5-chloro-2-(substituted phenylamino)-pyrimidin-4-ylamino]-2-methyl-3,4-dihydro-2H-isoquinolin-1-ones are prepared from 8-(2,5-dichloro-pyrimidin-4-ylamino)-2-methyl-3,4-dihydro-2H-isoquinolin-1-one and the corresponding aniline following the procedure of Example 1 or Example 20.

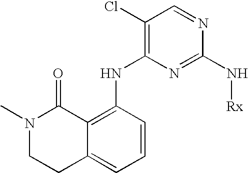

| | | Mass (m/z) | |
|---|---|---|---|
| Expl | | or Rf | |
| No. | Rx | (solvent) | NMR (400 MHz) δ (ppm) |
| 114 | 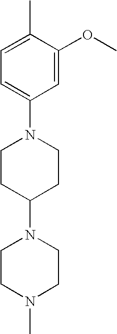 | 591, 593 | DMSO.d6: 1.46-1.59 (m, 2 H), 1.81-1.89 (m, 2 H), 2.15 (s, 3 H), 2.24-2.37 (m, 4 H), 2.63-2.73 (m, 2 H), 2.92- 2.98 (m, 2 H), 3.06 (s, 3 H), 3.35-3.41 (m, 1 H), 3.51- 3.57 (m, 2 H), 3.69-3.78 (m, 5 H), 6.49 (dd, 1 H), 6.63 (d, 1 H), 6.88 (d, 1 H), 7.23 (dd, 1 H), 7.37 (d, 1 H), 8.09 (s, 1 H), 8.14 (s, 1 H), 8.53-8.66 (m, 1 H), 12.58 (s, 1 H). |
| 115 | 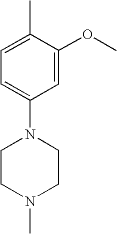 | 508 | CDCl3: 2.38 (s, 3 H), 2.57-2.67 (m, 4 H), 3.00 (t, 2 H), 3.14-3.22 (m, 7 H), 3.56 (t, 2 H), 3.87 (s, 3 H), 6.52 (dd, 1 H), 6.55 (d, 1 H), 6.82 (d, 1 H), 7.21 (s, 1 H), 7.35 (dd, 1 H), 8.08 (s, 1 H), 8.10 (d, 1 H), 8.72 (d, 1 H), 12.40 (s, 1 H). |
| 116 | 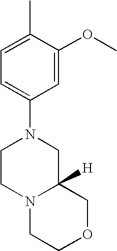 | 550 | DMSO-d6: 2.18-2.37 (m, 4 H), 2.66-2.72 (m, 1 H), 2.72- 2.86 (m, 2 H), 2.91-2.98 (m, 2 H), 3.06 (s, 3 H), 3.15- 3.22 (m, 1 H), 3.48-3.59 (m, 4 H), 3.61-3.68 (m, 1 H), 3.71-3.82 (m, 5 H), 6.48 (dd, 1 H), 6.44 (d, 1 H), 6.89 (d, 1 H), 7.23 (dd, 1 H), 7.40 (d, 1 H), 8.09 (s, 1 H), 8.14 (s, 1 H), 8.53-8.66 (m, 1 H), 12.59 (s, 1 H). |
| 117 | 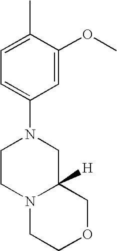 | 550, 552 | DMSO-d6: 2.18-2.37 (m, 4 H), 2.65-2.72 (m, 1 H), 2.72- 2.86 (m, 2 H), 2.91-2.98 (m, 2 H), 3.06 (s, 3 H), 3.14- 3.22 (m, 1 H), 3.47-3.58 (m, 4 H), 3.61-3.67 (m, 1 H), 3.71-3.81 (m, 5 H), 6.45-6.51 (m, 1 H), 6.62-6.66 (m, 1 H), 6.89 (d, 1 H), 7.18-7.27 (m, 1 H), 7.39 (d, 1 H), 8.09 (s, 1 H), 8.15 (s, 1 H), 8.53-8.64 (m, 1 H), 12.58 (s, 1 H). |
| 118 | 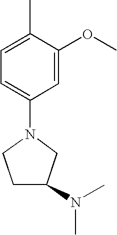 | 522, 524 | DMSO-d6: 1.77-1.89 (m, 1 H), 2.12-2.22 (m, 1 H), 2.22 (s, 6 H), 2.76-2.86 (m, 1 H), 2.94 (t, 2 H), 3.02-3.11 (m, 1 H), 3.22-3.34 (m, 1 H), 3.35-3.43 (m, 1 H), 3.43-3.50 (m, 1 H), 3.53 (t, 2 H), 3.75 (s, 3 H), 6.11 (dd, 1 H), 6.22 (d, 1 H), 6.86 (d, 1 H), 7.10-7.23 (m, 1 H), 7.25 (d, 1 H), 8.06 (s, 1 H), 8.10 (s, 1 H), 8.62 (br.s, 1 H), 12.56 (s, 1 H). |
| 119 | 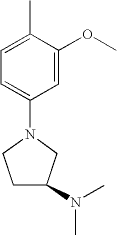 | 522 | DMSO-d6: 1.77-1.89 (m, 1 H), 2.12-2.22 (m, 1 H), 2.22 (s, 6 H), 2.77-2.86 (m, 1 H), 2.94 (t, 2 H), 3.03-3.12 (m, 4 H), 3.24-3.33 (m, 1 H), 3.35-3.43 (m, 1 H), 3.46-3.50 (m, 1 H), 3.54 (t, 2 H), 3.74 (s, 3 H), 6.11 (dd, 1 H), 6.22 (d, 1 H), 6.86 (d, 1 H), 7.11-7.25 (m, 1 H), 7.25 (d, 1 H), 8.06 (s, 1 H), 8.10 (s, 1 H), 8.61 (br.s, 1 H), 12.56 (s, 1 H). |
| 120 | 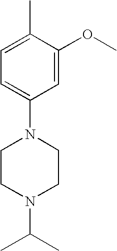 | 536, 538 | DMSO-d6: 1.02 (d, 6 H), 2.56-2.63 (m, 4 H), 2.63-2.74 (m, 1 H), 2.95 (t, 2 H), 3.06 (s, 3 H), 3.10-3.17 (m, 4 H), 3.54 (t, 2 H), 3.75 (s, 3 H), 6.48 (dd, 1 H), 6.63 (d, 1 H), 6.89 (d, 1 H), 7.22 (dd, 1 H), 7.38 (d, 1 H), 8.09 (s, 1 H), 8.15 (s, 1 H), 8.51-8.67 (m, 1 H), 12.58 (s, 1 H). |
| 121 | 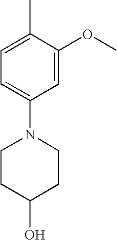 | 509, 511 | DMSO-d6: 1.45-1.57 (m, 2 H), 1.80-1.89 (m, 2 H), 2.79- 2.89 (m, 2 H), 2.95 (t, 2 H), 3.06 (s, 3 H), 3.48-3.58 (m, 4 H), 3.58-3.67 (m, 1 H), 3.75 (s, 3 H), 4.68 (d, 1 H), 6.49 (dd, 1 H), 6.63 (d, 1 H), 6.88 (d, 1 H), 7.23 (dd, 1 H), 7.37 (d, 1 H), 8.09 (s, 1 H), 8.12 (s, 1 H), 8.56-8.66 (m, 1 H), 12.58 (s, 1 H). |
| 122 | 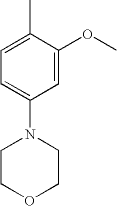 | 495 | DMS-d6: 2.95 (t, 2 H), 3.07 (s, 3 H), 3.10-3.16 (m, 4 H), 3.55 (t, 2 H), 3.73-3.82 (m, 7 H), 6.50 (dd, 1 H), 6.66 (d, 1 H), 6.89 (d, 1 H), 7.24 (dd, 1 H), 7.43 (d, 1 H), 8.10 (s, 1 H), 8.16 (s, 1 H), 8.56-8.66 (m, 1 H), 12.59 (s, 1 H). |
| 123 | 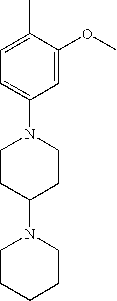 | 576, 578 | CDCl3: 1.40-1.52 (m, 2 H), 1.58-1.84 (m, 6 H), 1.84- 1.99 (m, 2 H), 2.35-2.47 (m, 1 H), 2.47-2.63 (m, 4 H), 2.63-2.74 (m, 2 H), 3.00 (t, 2 H), 3.19 (s, 3 H), 3.57 (t, 2 H), 3.63-3.70 (m, 2 H), 3.87 (s, 3 H), 6.52 (dd, 1 H), 6.55 (d, 1 H), 6.83 (d, 1 H), 7.21 (s, 1 H), 7.36 (dd, 1 H), 8.08 (s, 1 H), 8.08 (s, 1 H), 8.09 (dd, 1 H), 8.72 (d, 1 H), 12.39 (s, 1 H). |

### Example 124

The following 8-[5-Chloro-2-(substituted phenylamino)pyrimidin-4-ylamino]-2-ethyl-3,4-dihydro-2H-isoquinolin-1-ones are prepared from 8-(2,5-dichloro-pyrimidin-4-ylamino)-2-ethyl-3,4-dihydro-2H-isoquinolin-1-one and the corresponding aniline following the procedure of Example 1 or Example 20.

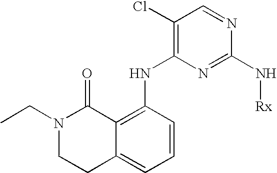

| | | Mass (m/z) | |
|---|---|---|---|
| Expl | | or Rf | |
| No. | Rx | (solvent) | NMR (400 MHz) δ (ppm) |
| 125 | 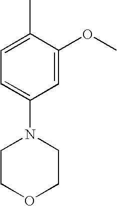 | 509, 511 | CDCl3: 1.24 (t, 3 H), 2.97 (t, 2 H), 3.09-3.15 (m, 4 H), 3.54 (t, 2 H), 3.65 (q, 2 H), 3.82-3.91 (m, 7 H), 6.50 (dd, 1 H), 6.53 (d, 1 H), 6.80-6.86 (m, 1 H), 7.24 (s, 1 H), 7.35 (dd, 1 H), 8.08 (s, 1 H), 8.14 (d, 1 H), 8.69 (d, 1 H), 12.36 (s, 1 H). |
| 126 | 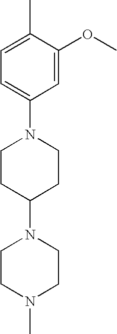 | 605, 607 | CDCl3: 1.24 (t, 3 H), 1.65-1.78 (m, 2 H), 1.93-1.98 (m, 2 H), 2.32 (s, 3 H), 2.35-2.74 (m, 11 H), 2.97 (t, 2 H), 3.54 (t, 2 H), 3.62-3.68 (m, 4 H), 3.86 (s, 3 H), 6.51 (dd, 1 H), 6.55 (d, 1 H), 6.83 (d, 1 H), 7.21 (s, 1 H), 7.35 (dd, 1 H), 8.08 (s, 1 H), 8.10 (d, 1 H), 8.69 (d, 1 H), 12.34 (s, 1 H). |

### Example 127

The following 5-chloro-N⁴-(1,1-dioxo-1λ⁶-thiochroman-8-yl)-N²-(2-substituted phenyl)-pyrimidine-2,4-diamines are prepared from (2,5-cichloro-pyrimidin-4-yl)-(1,1-dioxo-1λ⁶-thiochroman-8-yl)-amine and the corresponding aniline following the procedure of Example 20.

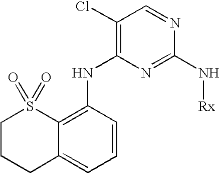

| Expl | | Mass (ESI) | HPLC |
|---|---|---|---|
| No. | Rx | m/z | Retention time (min) |
| 128 | 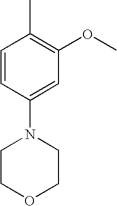 | 516 | 2.59 |
| 129 | 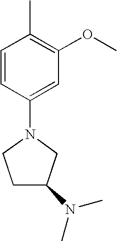 | 543 | 2.30 |
| 130 | 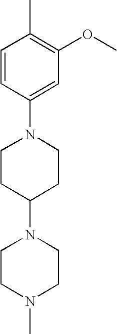 | 612 | 2.27 |
| 131 | 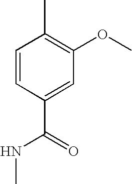 | 488 | 2.85 |

### Example 132

### Preparation of 5-chloro-N²-(2-methoxy-4-morpholin-4-yl-phenyl)-N⁴-[2-[2H-tetrazol-5-yl)-phenyl]-pyrimidine-2,4-diamine

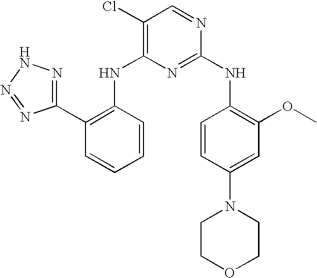

To a solution of 2-(2,5-dichloro-pyrimidin-4-ylamino)-benzonitrile (200 mg, 0.758 mmol) and 2-methoxy-4-morpholin-4-yl-phenylamine dihydrochloride (213 mg, 0.758 mmol) in 2-methoxyethanol (4 mL), 1N HCl/EtOH (1.5 ml) is added at room temperature. The mixture is heated at 100° C. for 15 h. The solvent is evaporated, and the mixture is purified by reverse phase HPLC to give 2-[5-chloro-2-(2-methoxy-4-morpholin-4-yl-phenylamino)-pyrimidin-4-ylamino]-benzonitrile (100 mg). MS (ESI) m/z 437, HPLC retention time 2.37 min.

To a solution of 2-[5-chloro-2-(2-methoxy-4-morpholin-4-yl-phenylamino)-pyrimidin-4-ylamino]-benzonitrile (75 mg, 0.172 mmol) and triethylamine hydrochloride (69.8 mg, 0.507 mmol) in toluene (3 ml), sodium azide (33.5 mg, 0.515 mmol) is added at room temperature. The mixture is refluxed for 15 h. The solvent is evaporated, and the mixture is purified by reverse phase HPLC to 5-chloro-N²-(2-methoxy-4-morpholin-4-yl-phenyl)-N⁴-[2-(2H-tetrazol-5-yl)-phenyl]-pyrimidine-2,4-diamine. MS (ESI) m/z 480, HPLC retention time 2.45 min.

The following compounds are prepared as described in Example I or Example 20.

### Example 133

### 5-Chloro-N²-{4-[4-(4-ethyl-piperazin-1-yl)-piperidin-1-yl]-2-methoxy-phenyl}-N⁴-[2-(propane-2-sulfonyl)-phenyl]-pyrimidine-2,4-diamine

The title compound is prepared using N-ethylpiperazin.

### Example 134

### 2-{5-Chloro-2-[4-((S)-3-ethylamino-pyrrolidin-1-yl)-2-methoxy-phenylamino]-pyrimidin-4-ylamino}-N-isopropyl-benzenesulfonamide

The title compound is prepared using ethyl-(S)-pyrrolidin-3-yl-amine.

### Example 135

### 2-{5-Chloro-2-[4-((R)-3-ethylamino-pyrrolidin-1-yl)-2-methoxy-phenylamino]-pyrimidin-4-ylamino}-N-isopropyl-benzenesulfonamide

The title compound is prepared using ethyl-(R)-pyrrolidin-3-yl-amine.

### Example 136

### 2-{5-Chloro-2-[2-methoxy-4-((S)-3-methylamino-pyrrolidin-1-yl)phenylamino]-pyrimidin-4-ylamino}-N-isopropyl-benzenesulfonamide

The title compound is prepared using methyl-(S)-pyrrolidin-3-yl-amine.

### Example 137

### 2-{5-Chloro-2-[2-methoxy-4-((R)-3-methylamino-pyrrolidin-1-yl)-phenylamino]-pyrimidin-4-ylamino}-N-isopropyl-benzenesulfonamide

The title compound is prepared using methyl-(R)-pyrrolidin-3-yl-amine.

### Example 138

### 2-{5-chloro-2-[4-((R)-3-dimethylamino-pyrrolidin-1-yl)-2-methoxy-phenylamino]-pyrimidin-4-ylamino}-N-isopropyl-benzenesulfonamide

The title compound is prepared using dimethyl-(R)-pyrrolidin-3-yl-amine.

### Example 139

### 2-{5-Chloro-2-[4-((S)-3-dimethylamino-pyrrolidin-1-yl)-2-methoxy-phenylamino]-pyrimidin-4-ylamino}-N-isopropyl-benzenesulfonamide

The title compound is prepared using dimethyl-(S)-pyrrolidin-3-yl-amine.

### Example 140

### 2-{5-Chloro-2-[2-ethoxy-4-(4-methyl-piperazin-1-yl)-phenylamino]-pyrimidin-4-ylamino}-N-isopropyl-benzenesulfonamide

The title compound is prepared starting from 5-Fluoro-2-nitrophenole and using iodo-ethane.

### Example 141

### 2-{5-Chloro-2-[2-isopropoxy-4-(4-methyl-piperazin-1-yl)-phenylamino]-pyrimidin-4-ylamino}-N isopropyl-benzenesulfonamide

The title compound is prepared starting from 5-Fluoro-2-nitrophenole and using 2-bromo-propane.

### Example 142

### 2-{5-Chloro-2-[2-cyclopropylmethoxy-4-(4-methyl-piperazin-1-yl)-phenylamino]-pyrimidin-4-ylamino}-N-isopropyl-benzenesulfonamide

The title compound is prepared starting from 5-Fluoro-2-nitrophenole and using bromomethyl-cyclopropane

Physicochemical Data:

| | | HPLC [t | (ESI+): | |
|---|---|---|---|---|
| Exam- | mp. | Ret min] | m/z | |
| ple | [° C.] | System 1 | (M + H)+ | Opt. Rotation; T = 20° C. |
| 133 | 123-125 | 3.90 | | Achiral, no optical rotation |
| 134 | 169-173 | 4.23 | 559.9 | α: −2.3° [c = 0.565, DMSO] |
| | | | | @589 nm |
| 135 | 169-173 | 4.23 | 559.9 | α: +1.6° [c = 0.50, DMSO] |
| | | | | @589 nm |
| 136 | 195-200 | 4.13 | 545.9 | Not measurable |
| 137 | 195-200 | 4.13 | 454.9 | Not measurable |
| 138 | 164-165 | 4.21 | 559.8 | α: +12.5 [c = 0.53, MeOH] |
| | | | | @589 nm |
| 139 | 162-164 | 4.22 | 559.9 | α: −14.5 [c = 0.525, DMSO] |
| | | | | @589 nm |
| 140 | 178-180 | 4.32 | | Achiral, no optical rotation |
| 141 | 189-191 | 4.50 | | Achiral, no optical rotation |
| 142 | 175-176 | 4.62 | | Achiral, no optical rotation |

### Analytical HPLC Conditions:

### System 1:

Linear gradient 20-100% CH₃CN (0.1% TFA) and H₂O (0.1% TFA) in 7 min+2 min 100% CH₃CN (0.1% TFA); detection at 215 nm, flow rate 1 mL/min at 30° C. Column: Nucleosil 100-3 C18 (125×4.0 mm)

### Intermediates

### Example I1

### Preparation of 2-(5-bromo-2-chloro-pyrimidin-4-ylamino)-N-methyl-benzenesulfonamide

A solution of 5-bromo-2,4-dichloropyrimidine (684 mg, 3.0 mmol) and 2-amino-N-methyl-benzenesulfonamide (559 mg, 3.0 mmol) in N,N-dimethylformamide (10 mL) containing potassium carbonate (830 mg, 6.0 mmol) is stirred at room temperature for 23 hours. Saturated aqueous ammonium chloride is added and the mixture is poured into water and extracted twice with ethyl acetate. The organic layer is washed with brine, dried over sodium sulfate, and evaporated in vacuo. The residue is purified with silica gel column chromatography (n-hexane-ethyl acetate gradient) to afford the title compound as a slightly yellow solid.

¹H-NMR (CDCl₃), δ (ppm): 2.67 (d, 3H), 4.79 (q, 1H), 7.26 (s, 1H), 7.29 (ddd, 1H), 7.66 (ddd, 1H), 7.95 (dd, 1H), 8.37 (s, 1H), 8.48 (d, 1H), 9.52 (s, 1H). Rf (n-hexane:ethyl acetate=10:3): 0.33.

### Example I2

### Preparation of 2-(2,5-Dichloro-pyrimidin-4-ylamino)-N-(2,2-dimethyl-propyl)-benzenesulfonamide

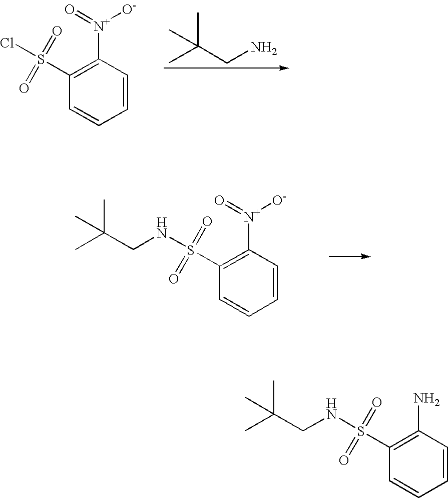

To a solution of 2-nitro-benzenesulfonyl chloride (5 g, 22.6 mmol), 2,2-dimethyl-propylamine (2.36 g, 27.1 mmol) in pyridine (25 ml) and dichloromethane (25 ml), a solution of 2-nitro-benzenesulfonyl chloride (5 g, 22.6 mmol) in dichloromethane (25 ml) was added dropwise at 0° C. After stirring for 18 h at room temperature, the reaction mixture was poured into water and extracted twice with dichloromethane. The organic layer was successively washed with 1M HCl, saturated aqueous NaHCO3, and brine, dried over magnesium sulfate, and evaporated in vacuo.

The residue was dissolved in AcOEt (100 ml). To the solution, tin chloride dehydrate (21.1 g, 93.8 mmol) was added at 80° C. After stirring for 18 h at 80° C., the reaction mixture was poured into 2M NaOH and extracted twice with ethyl acetate. The organic layer was successively washed with 1M NaOH, and brine, dried over magnesium sulfate, and evaporated in vacuo, to give the 2-Amino-N-(2,2-dimethyl-propyl)-benzenesulfonamide (4.15 g): MS (ESI) m/z 243, HPLC retention time 3.68 min.

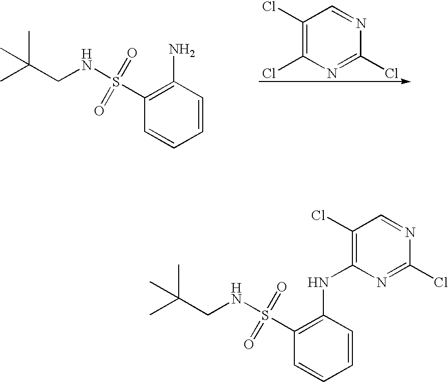

To a solution of 2-amino-N-(2,2-dimethyl-propyl)-benzenesulfonamide (1.20 g, 4.96 mmol) of N,N-dimethylformamide (10 mL), sodium hydride (496 g, 12.4 mmol) was added portionwise at 0° C. After stirring for 15 min, 2,4,5-trichloropyrimidine (1.36 g, 7.44 mmol) was added. The mixture was stirred at 0° C. for 30 minutes and was further stirred at room temperature for 7 hrs. The mixture was poured into water and extracted twice with ethyl acetate. The organic layer was washed with brine, dried over magnesium sulfate, and evaporated in vacuo. The residue was purified by silica gel column chromatography (n-hexane-ethyl acetate gradient) to afford the title compound (0.48 g): MS (ESI) m/z 389, HPLC retention time 4.27 min

### Example I3

### Preparation of 2-(5-Bromo-2-chloro-pyrimidin-4-ylamino)-N-isopropylbenzenesulfonamide

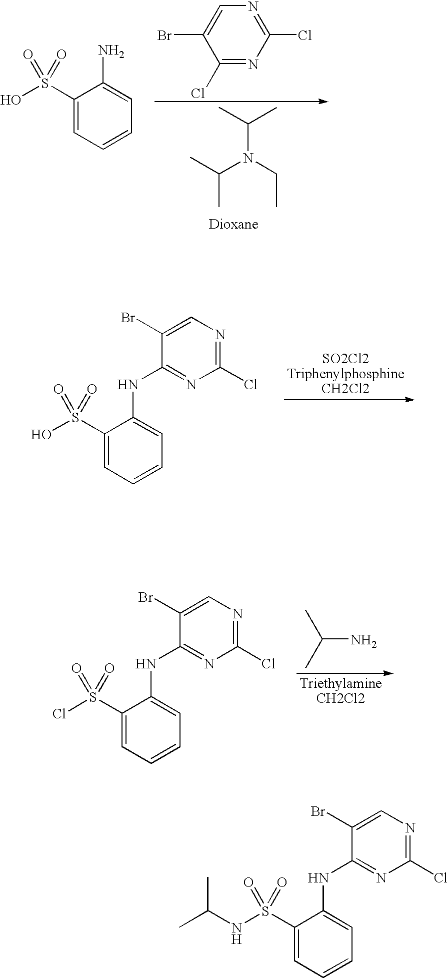

A mixture of orthanilic acid (0.10 mol), diisopropylethylamine (0.21 mol), and 5-bromo-2,4-dichloropyrimidine (0.11 mol) in dioxane (200 mL) is stirred and refluxed for 20 h. The reaction mixture is evaporated in vacuo to give crude 2-(5-bromo-2-chloropyrimidin-4-ylamino)benznenesulfonic acid.

To a solution of triphenylphosphine (0.20 mol) in CH₂Cl₂ (200 mL) is added sulfuryl chloride (0.20 mol) at −2° C. After stirring at 0-10° C. for 20 min, a solution of crude 2-(5-bromo-2-chloropyrimidin-4-ylamino)benzenesulfonic acid dissolved in CH₂Cl₂ (130 mL) is added to the reaction mixture at 15-25° C. over 10 min. The reaction mixture is stirred at room temperature for 24 h to afford a crude 2-(5-bromo-2-chloropyrimidin-4-ylamino)benzenesulfonyl chloride as CH₂Cl₂ solution, which is added to a solution of isopropylamine (0.40 mol) and triethylamine (0.20 mol) in CH₂Cl₂ (200 mL) at room temperature over 10 min. The reaction mixture is stirred at room temperature for 3 h and then 1N HCl (300 mL) is added. The organic layer is washed with 1N HCl and brine, dried over MgSO₄ and evaporated in vacuo. The resulting residue is purified by silica gel column chromatography to give 2-(5-bromo-2-chloro-pyrimidin-4-ylamino)-N-isopropylbenzenesulfonamide (0.062 mol) as white solid. MS: 407 [M+1]⁺, ¹H NMR (400 MHz, δ, ppm) CDCl₃: 1.05 (d, 6H), 3.46 (sep, 1H), 4.30 (d, 1H), 7.29 (dt, 1H). 7.66 (dt, 1H), 7.99 (dd, 1H), 8.40 (s, 1H), 8.44 (dd., 1H), 9.37 (s, 1H).

### Example I4

The following 2,5-dichloro-4-substituted pyrimidines are prepared by repeating the method described above by use of appropriate starting materials and conditions.

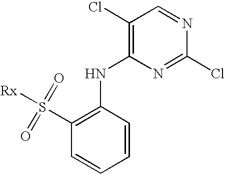

| Expl | | |
|---|---|---|
| No. | Rx | NMR (400 MHz) δ (ppm) |
| I5 | 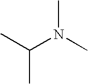 | DMSO-d6: 0.85 (d, 6 H), 1.57 (s, 3 H), 1.77-1.89 (m, 1 H), 2.73 (s, 3 H), 2.79 (d, 2 H), 7.28 (dd, 1 H), 7.65 (dd, 1 H), 7.85 (d, 1 H), 8.28 (s, 1 H), 8.55 (d, 1 H), 9.87 (brs, 1 H). |
| I6 | 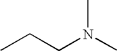 | DMSO-d6: 1.0 (d, 6 H), 1.56 (s, 3 H), 2.67 (s, 3 H), 4.15-4.24 (m, 1 H), 7.26 (dd, 1 H), 7.64 (dd, 1 H), 7.92 (d, 1 H), 8.28 (s, 1 H), 8.53 (d, 1 H), 9.73 (brs, 1 H). |
| I7 | 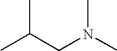 | DMSO-d6: 0.84 (t, 3 H), 1.48-1.57 (m, 2 H), 1.56 (s, 3 H), 2.74 (s, 3 H), 3.04 (dd, 2 H), 7.27 (dd, 1 H), 7.65 (dd, 1 H), 7.87 (d, 1 H), 8.28 (s, 1 H), 8.56 (d, 1 H), 9.84 (brs, 1 H). |
| I8 | 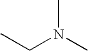 | DMSO-d6: 1.10 (t, 3 H), 2.75 (s, 3 H), 3.17 (q, 4 H), 7.27 (dd, 1 H), 7.65 (dd, 1 H), 7.88 (d, 1 H), 8.28 (s, 1 H), 8.57 (d, 1 H), 9.81 (brs, 1 H). |
| I9 | 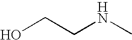 | CDCl3: 1.89 (t, 1 H), 3.11 (dd, 2 H), 3.61 (dd, 2 H), 5.25-5.18 (m, 1 H), 7.30 (d, 1 H), 7.74-7.38 (m, 1 H), 7.96 (dd, 1 H), 8.28 (s, 1 H), 8.48 (d, 1 H), 9.46 (s, 1 H). Rf 0.26 (Hexane/AcOEt = 1/1) |
| I10 | 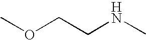 | CDCl3: 3.14-3.09 (m, 2 H), 3.16 (s, 3 H), 3.30- 3.28 (m, 2 H), 4.98 (t, 1 H), 7.31-7.27 (m, 1 H), 7.69-7.65 (m, 1 H), 7.96 (dd, 1 H), 8.30 (s, 1 H), 8.49 (dd, 1 H), 9.45 (s, 1 H). Rf 0.42 (Hexane/ AcOEt = 1/1) |
| I11 | 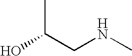 | CDCl3: 1.10 (d, 3 H), 1.91 (d, 1 H), 2.81-2.74 (m, 1 H), 3.09-3.03 (m, 1 H), 3.87-3.77 (m, 1 H), 5.25-5.18 (m, 1 H), 7.74-7.38 (m, 1 H), 7.97 (dd, 1 H), 8.30 (s, 1 H), 8.48 (d, 1 H), 9.46 (s, 1 H). Rf 0.40 (Hexane/AcOEt = 1/1) |
| I12 | 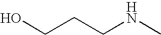 | CDCl3: 1.46 (t, 1 H), 1.67-1.60 (m, 2 H), 3.11 (dd, 2 H), 3.14-3.09 (m, 2 H), 5.45-5.42 (m, 1 H), 7.32-7.27 (m, 1 H), 7.69-7.64 (m, 1 H), 7.97 (dd, 1 H), 8.28 (s, 1 H), 8.46 (dd, 1 H), 9.46 (s, 1 H). Rf 0.28 (Hexane/AcOEt = 1/1) |
| I13 | 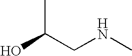 | CDCl3: 1.10 (d, 3 H), 1.91 (d, 1 H), 2.81-2.74 (m, 1 H), 3.09-3.03 (m, 1 H), 3.87-3.77 (m, 1 H), 5.25-5.18 (m, 1 H), 7.74-7.38 (m, 1 H), 7.97 (dd, 1 H), 8.30 (s, 1 H), 8.48 (d, 1 H), 9.46 (s, 1 H). Rf: 0.40 (Hexane/AcOEt = 1/1) |
| I14 | 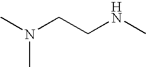 | CDCl3: 2.02 (s, 3 H), 2.04 (s, 3 H), 2.30 (t, 2 H), 2.97-2.94 (m, 2 H), 7.31-7.29 (m, 1 H), 7.39 (s, 1 H), 7.30 (dd, 1 H), 7.69-7.64 (m, 1 H), 7.97 (dd, 1 H), 8.28 (s, 1 H), 8.48 (d, 1 H), 9.49 (s, 1 H). Rf 0.05 (AcOEt) |
| I15 | 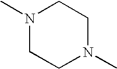 | CDCl3: 2.23 (s, 3 H), 2.40 (t, 4 H), 3.11-3.08 (m, 4 H), 7.31-7.26 (m, 1 H), 7.84 (dd, 1 H), 8.28 (s, 1 H), 8.61 (dd, 1 H), 9.79 (s, 1 H). Rf 0.13 (AcOEt) |
| I16 | 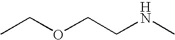 | CDCl3: 1.04 (t, 3 H), 3.12 (dd, 2 H), 3.34-3.27 (m, 4 H), 5.01 (t, 1 H), 7.32-7.26 (m, 1 H), 7.69- 7.65 (m, 1 H), 7.98 (d, 1 H), 8.29 (s, 1 H), 8.51 (dd, 1 H), 9.48 (s, 1 H). Rf 0.45 (Hexane/ AcOEt = 1/1) |
| I17 | 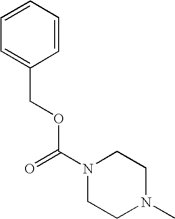 | CDCl3: 3.13-2.98 (m, 4 H), 3.53 (t, 4 H), 5.06 (s, 2 H), 7.37-7.27 (m, 6 H), 7.71-7.67 (m, 1 H), 7.83 (dd, 1 H), 8.30 (s, 1 H), 8.64 (dd, 1 H), 9.76 (s, 1 H). Rf 0.34 (Hexane/AcOEt = 3/1) |

### Example I18

### Preparation of 8-(2,5-dichloro-pyrimidin-4-ylamino)-2-methyl-3,4-dihydro-2H-isoquinolin-1-one

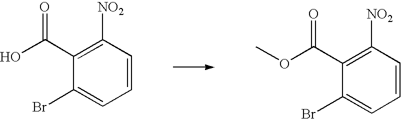

To a solution of 2-Bromo-6-nitro-benzoic acid (33 g, 134 mmol) in MeOH (250 mL), is added cesium carbonate (22 g, 67 mmol) at room temperature and the mixture is stirred at room temperature for 20 minutes. The reaction mixture is evaporated to give a residue. The residue is dissolved in DMF (300 ml) and iodomethane (10 mL, 161 mmol) is added to the mixture at 0° C.

The mixture is stirred at room temperature for 14 hours. Addition of water (500 mL) gives precipitates which are filtered and washed with water to give 2-Bromo-6-nitro-benzoic acid methyl ester (34 g) in quantitative yield. ¹H-NMR (400 MHz, δ, ppm) CDCl3: 4.02 (s, 3H), 7.48 (dd, 1H), 7.92 (dd, 1H), 8.18 (dd, 1H).

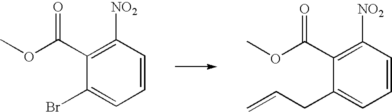

To a solution of 2-bromo-6-nitro-benzoic acid methyl ester (32.7 g, 126 mmol) in toluene (420 mL) are added tetrakis(triphenylphosphine) palladium (0) (3.6 g, 3.1 mmol) and allyltributyltin (45.8 g, 138 mmol) and the reaction mixture is stirred at 110° C. for 20 hours. The mixture is cooled to room temperature and 4% CsF water solution (400 mL) is added into the mixture. The mixture is filtered through Celite™ and extracted with EtOAc. The combined organic phases are washed with brine, dried over Na2SO4 and concentrated under reduced pressure. Purification by silica gel flash chromatography eluting with Hexane/EtOAc (95:5) gives 2-allyl-6-nitro-benzoic acid methyl ester as a yellow oil (28 g, quantitative yield). ¹H-NMR (400 MHz, δ, ppm) CDCl3: 3.48 (d, 2H), 3.94 (s, 3H), 5.07-5.17 (m, 2H), 5.88 (ddt, 1H), 7.52 (dd, 1H), 7.58 (dd, 1H), 8.02 (dd, 1H).

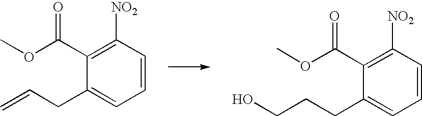

To a solution of 2-allyl-6-nitro-benzoic acid methyl ester (9.5 g, 43 mmol) in THF (100 mL) is added neat borane-methyl sulfide (43 mL, 86 mmol) at 0° C., and the mixture allows to stir at room temperature for 4 hours. On cooling, 1N NaOH (300 mL) is added followed by 30% hydrogenperoxide (150 mL). The resulting mixture is allowed to reach room temperature and stirred for 1 hour. The reaction is then worked up by diluting with water and extracting with EtOAc. The combined extracts are washed sequentially with water and brine, dried over Na2SO4, filtered, concentrated, and purified on silica gel with a gradient of 50% EtOAc/Hexane to provide 9.2 g of 2-(3-Hydroxy-propyl)-6-nitro-benzoic acid-methyl ester in 90% yields. ¹H-NMR (400 MHz, δ, ppm) CDCl3: 1.90 (dddd, 2H), 2.80 (dd, 2H), 3.64 (dd, 2H), 3.98 (s, 3H), 7.52 (dd, 1H), 7.61 (dd, 1H), 8.02 (dd, 1H).

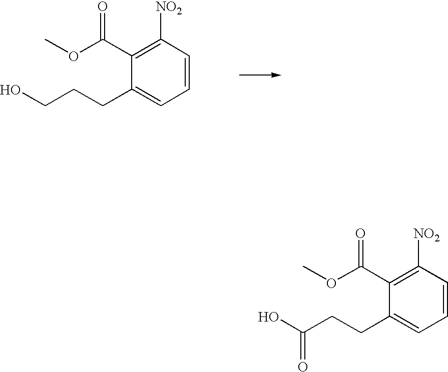

A solution of 2-(3-Hydroxy-propyl)-6-nitro-benzoic acid methyl ester (23.6 g, 99 mol) in acetone (500 mL) was treated with Jones reagent (CrO3/H2SO4, 58 mL) at 0° C. to room temperature for 4 hours. The reaction mixture is quenched with isopropyl alcohol (30 mL) and water (300 mL), and concentrated. The resulting mixture is extracted with EtOAc. The combined organic phases are extracted with 1N NAOH (250 mL×2) and then the aqueous phases are acidified with 6N HCl, and extracted with EtOAc. The organic layer is washed with brine, dried over Na2SO4, filtered, and concentrated to give 20 g (80%) of 2-(2-carboxy-ethyl)-6-nitro-benzoicacid methyl ester as a brown solid. ¹H-NMR (400 MHz, δ, ppm) CDCl3: 2.73 (dd, 2H), 3.03 (dd, 2H), 3.97 (s, 3H), 7.53 (dd, 1H), 7.63 (d, 1H), 8.04 (d, 1H).

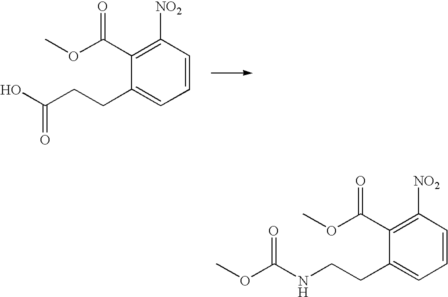

Diphenylphosphoryl azide (3.3 mL, 15.2 mmol) and triethylamine (2.12 mL, 15.2 mmol) are added to a solution of 2-(2-carboxy-ethyl)-6-nitro-benzoicacid methyl ester (3.5 g, 13.8 mmol) in dry toluene (130 mL) and the mixture is heated at 80° C. for 2 hours. To the mixture, copper(II) chloride (105 mg, 1.014 mmol) and anhydrous methanol (25 mL) are added and the mixture is heated at 80° C. for 2 hours and then cooled. The solution is successively washed with saturated sodium bicarbonate and water. The organic extracts are dried, filtered and concentrated. Purification by flash chromatography eluting with Hexane/EtOAc 2-(2-Methoxycarbonylamino-ethyl)-6-nitro-benzoicacid methylester as a yellow oil (2.7 g, 68%). ¹H-NMR (400 MHz, δ, ppm) CDCl3: 2.89 (dd, 2H), 3.42-3.49 (m, 2H), 3.65 (s, 3H), 3.97 (s, 3H), 4.99 (bs, 1H), 7.55 (dd, 1H), 7.59-7.63 (m, 1H), 8.04 (d, 1H).

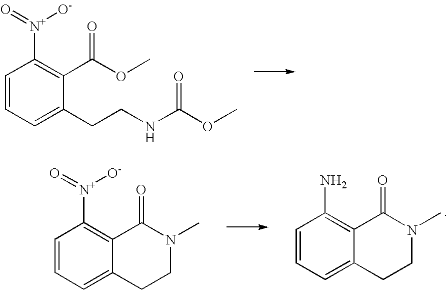

To a solution of 540 mg (1.91 mmol) of 2-(2-Methoxycarbonylamino-ethyl)-6-nitro-benzoic acid methyl ester in 15 mL of THF is added NaH (55%, 167 mg, 3.83 mmol) at 0° C. and stirred at the same temperature for 20 minutes. To the reaction mixture, iodomethane (544 mg 3.83 mmol) is added at the room temperature. After the reaction mixture is stirred at the room temperature for 30 minutes, aqueous NaHCO₃ solution is added. The resulting mixture is extracted with ethyl acetate and then the organic layer is washed with brine, dried over sodium sulfate, filtered and evaporated in vacuo to afford the crude residue (450 mg). ¹H-NMR (400 MHz, δ, ppm) CDCl3: 3.03 (dd, 2H), 3.15 (s, 3H), 3.62 (dd, 2H), 7.33-7.39 (m, 2H), 7.49 (dd, 1H).

To a solution of the crude material in 15 ml of EtOH, 1N HCl (3.8 ml, 3.8 mmol) and iron powder (533 mg, 9.55 mmol) are added and stirred at 60° C. for 1.5 hours. To the reaction mixture, 1N NaOH (4 ml, 8 mmol) and celite are added at 0° C. then filtrated with celite pad. The filtrate is concentrated in vacuo, then extracted with ethyl acetate. The organic layer is washed with brine, dried over sodium sulfate, filtered and evaporated in vacuo. The residue is purified with silica gel column chromatography (n-hexane:ethyl acetate=5:1 to 1:1) to afford the 8-Amino-2-methyl-3,4-dihydro-2H-isoquinolin-1-one (200 mg, 1.13 mmol, 59%). ¹H-NMR (400 MHz, δ, ppm) CDCl3: 2.90 (dd, 2H), 3.11 (s, 3H), 3.49 (dd, 2H), 6.10-6.40 (m, 2H), 6.41 (dd, 1H), 6.55 (d, 1H), 7.10 (dd, 1H).

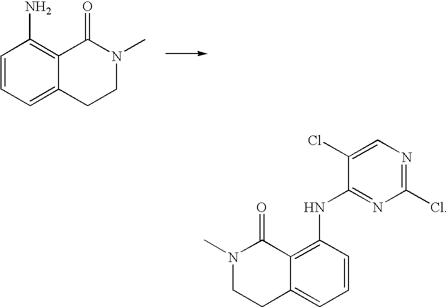

To a suspension of 8-Amino-2-methyl-3,4-dihydro-2H-isoquinolin-1-one (100 mg, 0.567 mmol) and K₂CO₃ (118 mg, 0.85 mmol) in 15 ml of dimethyl sulfoxide are added 2,4,5-trichlropyrimidine (156 mg, 0.85 mmol) and stirred at 60° C. for 20 hours. The reaction mixture is poured into water then the resulting precipitate is collected by filtration. The obtained solid is washed with ether:hexane=4:1 then dried under reduced pressure to give 8-(2,5-dichloro-pyrimidin-4-ylamino)-2-methyl-3,4-dihydro-2H-isoquinolin-1-one (120 mg, 0.37 mmol, 66%) as white solid. ¹H-NMR (400 MHz, δ, ppm) CDCl3: 3.02 (dd, 2H), 3.19 (s, 3H), 3.58 (dd, 2H), 6.91 (d, 1H), 7.47 (dd, 1H), 8.21 (s, 1H), 8.74 (d, 1H), 13.08 (s, 1H).

### Example I19

### Preparation of 7-(2,5-Dichloro-pyrimidin-4-ylamino)-2-methyl-2,3-dihydro-isoindol-1-one

N-Methyl-7-nirto-2,3-dihydroisoindole-1-one. At room temperature, a solution of methyl 2-bromomethyl-6-nitrobenzoate (1.26 g, 4.63 mmol) in THF (13 mL) is treated with 2M soln. of methylamine in THF (14 mL), stirred for 5 h, diluted with EtOAc (100 mL), washed with sat. aqueous solution of NaHCO₃ (15 mL) and brine (15 mL), dried (MgSO₄), and evaporated. A flash chromatography (30 g of silica gel; CH₂Cl₂/EtOAc 1:1) gives N-Methyl-7-nirto-2,3-dihydroisoindole-1-one (0.561 g, 2.92 mmol) in 63%. Yellow solid. R_{f} (CH₂Cl₂/EtOAc 1:1) 0.46. ¹H-NMR (400 MHz, CDCl₃) 3.21 (s), 4.44 (s), 7.63-7.69 (m, 2H), 7.70-7.75 (m, 1H). 7-Amino-N-methyl-2,3-dihydroisoindole-1-one. At room temperature, a solution of N-Methyl-7-nirto-2,3-dihydroisoindole-1-one (561.0 mg, 2.92 mmol) in EtOAc (8.4 mL) is treated with SnCl₂.2H₂O (2.68 g), stirred at 80° C. under reflux for 5 h, and treated with 30 mL of 5N NaOH at 0° C. After the both layers are separated, the aqueous layer is extracted with EtOAc (2×8 mL), the combined extracts are washed with brine (5 mL), dried (MgSO₄), and evaporated to give 7-Amino-N-methyl-2,3-dihydroisoindole-1-one (455.9 g, 2.81 mmol) in 96%. Yellow solid. R_{f} (CH₂Cl₂/EtOAc 1:1) 0.53. ¹H-NMR (400 MHz, CDCl₃) 3.12 (s), 4.28 (s), 5.20 (br. s), 6.56 (d, J=8.0), 6.68 (d, J=8.0), 7.21 (dd, J=8.0, 8.0).

7-(4-Amino-2,5-dichloropyrimidin-4-yl)amino-N-methyl-2,3-dihydroisoindole-1-one. At 0° C., a solution of 7-Amino-N-methyl-2,3-dihydroisoindole-1-one (232.6 mg, 1.43 mmol) in DMF (2.0 mL) is treated with 60% NaH (89.8 mg), stirred at the same temperature for 1.5 h, treated with a solution of 2,4,5-trichlropyrimidine (0.557 g) in DMF (3.5 mL), stirred for 1 h, and warmed to room temperature. After furthermore stirring for 13 h, the mixture is treated with sat. aqueous NH₄Cl (6 mL), and the resulting brown precipitates are collected by a filtration, followed by washing with H₂O, hexane, and CH₃CN to give 7-(4-Amino-2,5-dichloropyrimidin-4-yl)amino-N-methyl-2,3-dihydroisoindole-1-one (130.2 g, 0.416 mmol) in 26%. Brown solid. R_{f} (CH₂Cl₂/EtOAc 1:1) 0.50. ¹H-NMR (400 MHz, CDCl₃): 3.22 (s), 4.43 (s), 7.15 (d, J=8.0), 7.59 (dd, J=8.0, 8.0), 8.24 (s), 8.71 (d, J=8.0), 11.05 (br. s).

### Example I20

### Preparation of (2,5-Dichloro-pyrimidin-4-yl)-(1,1-dioxo-1λ⁶-thiochroman-8-yl)-amine

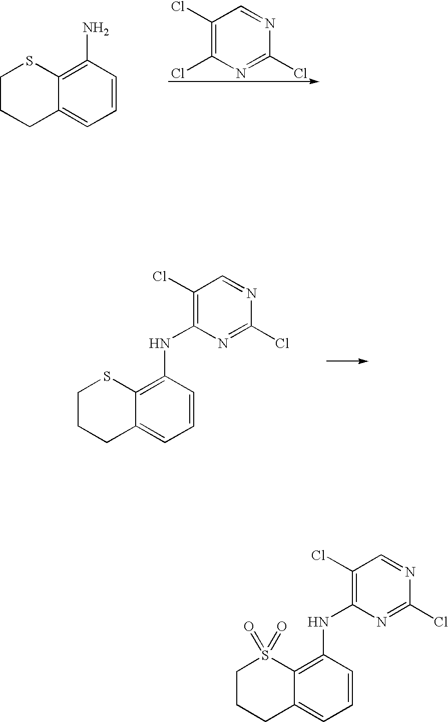

K₂CO₃ (543 mg, 3.94 mmol) was added to a solution of thiochroman-8-ylamine (500 mg, 3.03 mol) and 2,4,5-trichloro-pyrimidine (664 mg, 3.63 mmol) in DMF (5 ml). After stirring at 50° C. for 18 h, the mixture was poured into water and extracted twice with ethyl acetate. The organic layer was washed with brine, dried over magnesium sulfate, and evaporated in vacuo. The residue was purified by silica gel column chromatography (n-hexane-ethyl acetate gradient) to afford (2,5-dichloro-pyrimidin-4-yl)-thiochroman-8-yl-amine (200 mg). MS (ESI) m/z 312, HPLC retention time 4.00 min.

Sodium perborate tetrahydrate (443 mg, 2.88 mol) was added to a solution of (2,5-dichloro-pyrimidin-4-yl)-thiochroman-8-yl-amine (180 mg, 0.577 mol) in AcOH (4 ml). After stirring at 55° C. for 3 h, the mixture was poured into water and extracted twice with ethyl acetate. The organic layer was washed with brine, dried over magnesium sulfate, and evaporated in vacuo to afford (2,5-dichloro-pyrimidin-4-yl)-(1,1-dioxo-1λ⁶-thiochroman-8-yl)-amine (190 mg). MS (ESI) m/z 344, HPLC retention time 3.35 min.

### Example I21

### Preparation of 2-(2,5-Dichloro-pyrimidin-4-ylamino)-N-methyl-5-(4-methyl-piperazin-1-yl)-benzamide

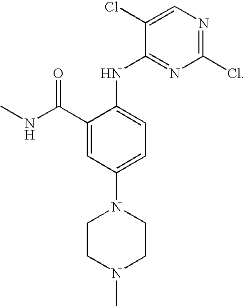

To a solution of 5-fluoro-2-nitro-benzoic acid (10 g, 54 mmol) and oxalyl chloride (6.1 mL, 70.2 mmol) in dichloromethane (300 mL), N,N-dimethylformamide (80 μL) is added and the mixture is stirred for 1 h at 50° C. The solvent is removed under reduced pressure and the residue is dissolved in THF. To the solution, 2N methylamine solution in THF is added at 0° C. and the mixture is stirred at room temperature for 15 hours. After addition of sat. sodium hydrogen carbonate aq., the mixture is extracted with ethyl acetate and the combined organic layer is washed with brine, dried over sodium sulfate, and concentrated in vacuo to give 5-fluoro-N-methyl-2-nitro-benzamide as a phase yellow solids (10.5 g, 98%).

To a suspension of potassium carbonate (15 g, 106 mmol) in N,N-dimethylformamide (250 mL), a solution of 5-fluoro-N-methyl-2-nitro-benzamide (10.5 g, 53 mmol) and N-methylpiperazine in N,N-dimethylformamide are added and the mixture is stirred at 60° C. for 15 hours. The insoluble materials are filtered off and washed with ethyl acetate. The filtrate is concentrated in vacuo to give N-methyl-5-(4-methyl-piperazin-1-yl)-2-nitro-benzamide (12.1 g, 82%).

After reduction of the nitro group of N-methyl-5-(4-methyl-piperazin-1-yl)-2-nitro-benzamide by use of palladium on charcoal under hydrogen atmosphere, 2,4,5-trichloropyrimidine (1.38 g, 7.52 mmol) is added to a solution of the resulting 2-amino-N-methyl-5-(4-methyl-piperazin-1-yl)-benzamide (934 mg, 3.76 mmol) and diisopropylethylamine (644 μL, 3.76 mmol) in ethyl acetate and the mixture is stirred at 60° C. After 1 hour, the mixture is cooled to 0° C. and triethylamine is added. The mixture is purified by silica gel column chromatography (CH2Cl2: MeOH=9:1) to give 2-(2,5-dichloro-pyrimidin-4-ylamino)-N-methyl-5-(4-methyl-piperazin-1-yl)-benzamide (368 mg, 25%). ¹H-NMR (400 MHz, δ, ppm) DMSO-d6: 2.29 (s, 3H), 2.52-2.59 (m, 4H), 2.79 (d, 3H), 3.20-3.27 (m, 4H), 7.20 (dd, 1H), 7.29 (d, 1H), 8.29 (d, 1H), 8.38 (s, 1H), 8.81-8.88 (m, 1H), 11.76 (s, 1H).

By repeating the procedure above, the following pyrimidine compounds are prepared by using appropriate starting materials and conditions.

| Expl | | |
|---|---|---|
| No. | Structures | NMR (400 MHz) δ (ppm) |
| I22 | 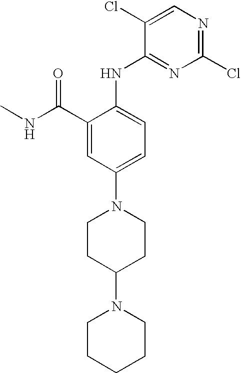 | DMSO: 1.36-1.99 (m, 10 H), 2.64-2.75 (m, 3 H), 2.79 (d, 3 H), 3.11-3.55 (m, 4 H), 3.77-3.91 (m, 2 H), 7.19 (dd, 1 H), 7.26-7.31 (m, 1 H), 8.29 (d, 1 H), 8.38 (s, 1 H), 8.80-8.92 (m, 1 H), 11.78 (s, 1 H). |
| I23 | 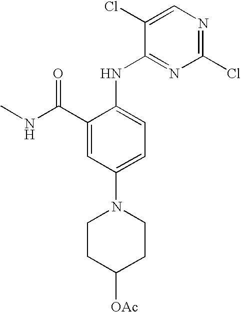 | DMSO: 1.63-1.72 (m, 2 H), 1.92-1.99 (m, 2 H), 2.03 (s, 3 H), 2.80 (d, 3 H), 3.02-3.11 (m, 2 H), 3.50-3.59 (m, 2 H), 4.80-4.89 (m, 1 H), 7.21 (dd, 1 H), 7.29 (d, 1 H), 8.30 (d, 1 H). 8.39 (s, 1 H), 8.74-8.83 (m, 1 H), 11.77 (s, 1 H). |
| I24 | 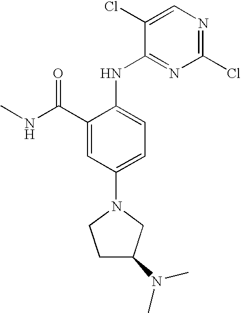 | CDCl3: 1.90-2.10 (m, 1 H), 2.20-2.82 (m, 1 H), 2.33 (s, 6 H), 2.83-2.92 (m, 1 H), 3.03 (d, 3 H), 3.14-3.19 (m, 1 H), 3.29-3.37 (m, 1 H), 3.41- 3.51 (m, 2 H), 6.24-6.31 (m, 1 H), 6.52 (d, 1 H), 6.73 (dd, 1 H), 8.10 (s, 1 H), 8.358 (d, 1 H), 10.85 (s, 1 H). ESI-MS m/z: 409 [M + 1]⁺ |

### Example I25

### Preparation of (S)-1-(3-Methoxy-4-nitro-phenyl)-3-methyl-piperidine-3-carboxylic Acid Ethyl Ester

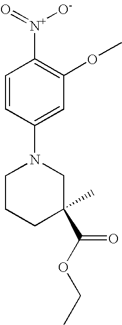

To a suspension of (S)-3-Methyl-piperidine-3-carboxylic acid ethyl ester (323.6 mg, 1.89 mmol) and potassium carbonate (313.2 mg g, 2.27 mmol) in N,N-dimethylformamide (3.0 mL), 4-fluoro-2-methoxy-1-nitro-benzene (388.1 mg, 2.27 mmol) is added and the mixture is stirred at 70° C. for 5 hours. The mixture is poured into water and extracted twice with ethyl acetate. The organic layer is successively washed with water and brine, dried over magnesium sulfate, and evaporated in vacuo. The residue is purified with silica gel column chromatography (n-hexane: ethyl acetate=5:1 to 4:1) to afford (S)-1-(3-Methoxy-4-nitro-phenyl)-3-methyl-piperidine-3-carboxylic acid ethyl ester (348.5 mg) as yellow oil in 57% yield.

Rf=0.50 (ether/Hexane=1/5). ¹H-NMR (400 MHz, CDCl₃, δ, ppm): (t, 3H), 1.45-1.36 (m, 1H), 1.80-1.67 (m, 2H), 2.33-2.27 (m, 1H), 2.87 (d, 1H), 3.02-2.96 (m, 1H), 3.65-3.60 (m, 1H), 3.96 (s, 3H), 4.16-4.10 (m, 2H), 6.41 (d, 1H), 6.48 (dd, 1H), 7.99 (d, 1H).

### Preparation of (S)-1-(3-Methoxy-4-nitro-phenyl)-3-methyl-piperidine-3-carboxylic Acid

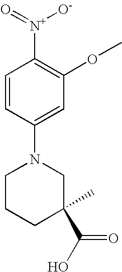

To a solution of (S)-1-(3-Methoxy-4-nitro-phenyl)-3-methyl-piperidine-3-carboxylic acid ethyl ester (348.5 mg, 1.08 mmol) in ethanol (2.0 mL), 5N sodium hydroxide (1.0 mL) is added and the mixture is stirred at room temperature for 5 hours. After the mixture is concentrated, 1N hydrogen chloride aq. is added and then extracted twice with ethyl acetate. The organic layer is successively washed with water and brine, dried over magnesium sulfate, and evaporated in vacuo to afford (S)-1-(3-Methoxy-4-nitro-phenyl)-3-methyl-piperidine-3-carboxylic acid as yellow oil in quantitative yield (317.9 g).

Rf=0.50 (AcOEt). ¹H-NMR (400 MHz, CDCl₃, δ, ppm): (t, 3H), 1.45-1.36 (m, 1H), 1.80-1.67 (m, 2H), 2.33-2.27 (m, 1H), 2.87 (d, 1H), 3.02-2.96 (m, 1H), 3.65-3.60 (m, 1H), 3.96 (s, 3H), 3.95 (s, 3H), 6.44 (d, 1H), 6.48 (dd, 1H), 7.97 (d, 1H).

### Preparation of (S)-1-(3-Methoxy-4-nitro-phenyl)-3-methyl-piperidine-3-carboxylic Acid Amide

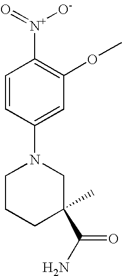

To a suspension of (S)-1-(3-Methoxy-4-nitro-phenyl)-3-methyl-piperidine-3-carboxylic acid (317.9 mg, 1.08 mmol) in dichloromethane (3.0 mL), oxalyl chloride (115 μL, 1.3 mmol) and N,N-dimethylformamide (1 drop) are added at 0° C. and the mixture is stirred at room temperature for 1.5 hours. After addition of 0.5N ammonia/dioxane solution at 0° C. and the mixture is further stirred at room temperature for 5.0 hours. After partition between dichloromethane and aqueous solution twice, the combined organic layer is successively washed with water and brine, dried over magnesium sulfate, and evaporated in vacuo to afford (S)-1-(3-Methoxy-4-nitro-phenyl)-3-methyl-piperidine-3-carboxylic acid amide (316.8 mg) as yellow amorphous solids in quantitative yield

Rf=0.50 (AcOEt). ¹H-NMR (400 MHz, CDCl₃, δ, ppm): 1.27 (s, 3H), 1.61-1.50 (m, 1H), 1.50-1.41 (m, 2H), 1.85-1.74 (m, 2H), 2.33-2.25 (m, 1H), 3.01-2.93 (m, 1H), 3.66-3.60 (m, 1H), 3.98 (s, 3H), 5.50-5.26 (m, 1H), 6.17-5.93 (m, 1H), 6.51 (s, 1H), 6.54-6.51 (m, 1H), 8.00 (dd, 1H).

### Example I26

### Preparation of (S)-1-(4-Amino-3-methoxy-phenyl)-3-methyl-piperidine-3-carboxylic Acid Amide

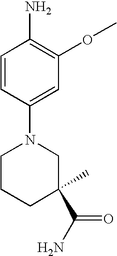

To a solution of (S)-1-(3-Methoxy-4-nitro-phenyl)-3-methyl-piperidine-3-carboxylic acid amide (316.8 mg, 1.08 mmol) in ethanol, 5% palladium on carbon is added under a nitrogen atmosphere. The reaction vessel is fitted with a balloon adapter and charged with hydrogen and evacuated three times until the reaction vessel is under a hydrogen atmosphere. The reaction is allowed to stir overnight. The reaction mixture is filtered through a pad of Celite and washed with methanol. The filtrate is concentrated in vacuo to afford (S)-1-(4-Amino-3-methoxy-phenyl)-3-methyl-piperidine-3-carboxylic acid amide (260.0 mg) as dark black amorphous solids in 91% yield.

### Example I27

### Preparation of 4-Amino-3-methoxy-N-methyl-benzamide

### Example I28

### Preparation of 3-methoxy-N-methyl-4-nitro-benzamide

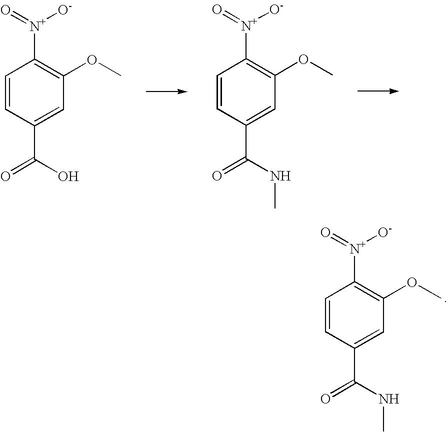

To a solution of 3-methoxy-4-nitro-benzoic acid (2.00 g, 10.2 mmol), HOBt (2.07 g, 15.3 mmol), and methylamine hydrochloride (0.891 g, 13.2 mmol) in DMF (20 ml), WSCl (2.38 g, 15.3 mmol) was added at room temperature. After stirring for 18 h at room temperature, the reaction mixture was poured into water and extracted twice with ethyl acetate. The organic layer was successively washed with 1M HCl, saturated aqueous NaCO₃ and brine, dried over magnesium sulfate, and evaporated in vacuo, to give the 3-methoxy-N-methyl-4-nitro-benzamide (1.53 g). MS (ESI) m/z 211, HPLC retention time 2.20 min.

To a solution of 3-methoxy-N-methyl-4-nitro-benzamide (1.5 g, 7.14 mmol), in AcOEt (75 ml), tin chloride dehydrate (8.06 g, 35.7 mmol) was added at 80° C. After stirring for 18 h at 80° C., the reaction mixture was poured into 2M NaOH and extracted twice with ethyl acetate. The organic layer was successively washed with 1M NaOH, and brine, dried over magnesium sulfate, and evaporated in vacuo, to give 4-amino-3-methoxy-N-methyl-benzamide (0.515 g). MS (ESI) m/z 181, HPLC retention time 1.38 min.

### Example I29

### Preparation of 3-Amino-4-methoxy-N-methyl-benzamide

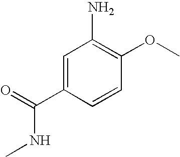

To a solution of 4-Methoxy-N-methyl-3-nitro-benzamide (1.5 g, 7.14 mmol), in AcOEt (75 ml), tin chloride dehydrate (8.06 g, 35.7 mmol) was added at 80° C. After stirring for 18 h at 80° C., the reaction mixture was poured into 2M NaOH and extracted twice with ethyl acetate. The organic layer was successively washed with 1M NaOH, and brine, dried over magnesium sulfate, and evaporated in vacuo, to give the title product (0.672 g). MS (ESI) m/z 181, HPLC retention time 1.07 min.

### Example I30

### Preparation of 7-Methoxy-4-methyl-6-nitro-4H-benzo[1,4]oxazin-3-one

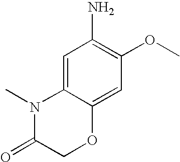

To a solution of 7-methoxy-4H-benzo[1,4]oxazin-3-one (1.9 g, 10.6 mmol) in AcOH (20 mL), is added dropwise fuming HNO₃ (13.7 mL) below 10° C. After stirred for 3 h, the reaction mixture is poured into ice cold water and the resulting white solids are collected by filtration. The solids are washed with H₂O and hexane and dried in vacuo to give 7-methoxy-6-nitro-4H-benzo[1,4]oxazin-3-one (1.4 g, 59%).

NaH (0.13 g, 5.4 mmol) is added to a suspension of 7-methoxy-6-nitro-4H-benzo[1,4]oxazin-3-one (1.4 g, 6.3 mmol) in DMF (20 mL) at 0° C. After stirred at room temperature for 1 h, MeI (0.95 g 6.8 mmol) is added to the reaction mixture at 0° C. The reaction mixture is stirred at room temperature overnight and quenched by H₂O at 0° C. Pale yellow solids are collected by filtration. The resulting solids are washed with H₂O and dried in vacuo to give 7-methoxy-4-methyl-6-nitro-4H-benzo[1,4]oxazin-3-one (0.98 g, 63%).

SnCl₂-2H₂O (4.5 g, 20 mmol) is added to a solution of 7-methoxy-4-methyl-6-nitro-4H-benzo[1,4]oxazin-3-one (0.98 g, 4.1 mmol) in AcOEt. After stirring at 80° C. for 3 h, the reaction mixture is cooled to room temperature. The solution is basified with 2N NaOH and extracted with AcOEt. The organic layer is washed with saturated NaHCO₃ and H₂O. The resulting solution is dried and evaporated in vacuo to give 6-amino-7-methoxy-4-methyl-4H-benzo[1,4]oxazin-3-one (0.86 g, >99%).

¹H-NMR CDCl3: 3.29 (s, 3H), 3.82 (s, 3H), 4.52 (s, 2H), 6.39 (s, 1H), 6.52 (s, 1H). Rf value: 0.20 (hexane:AcOEt=1:1).

### Example I31

### Preparation of 2,4-dimethoxy-5-(2-morpholin-4-yl-ethoxy)-phenylamine

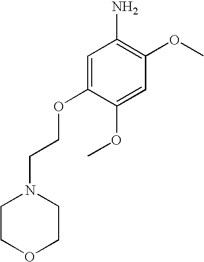

To a solution of 2,4-dimethoxy-5-nitro-phenol (2.8 g, 14.1 mmol) in DMF, are added 4-(2-Chloroethyl)morpholine hydrochloride (3.2 g, 17 mmol), K₂CO₃ (5.8 g, 42 mmol) and Kl (7.6 g, 42 mmol). After stirring at 80° C. overnight, the reaction mixture is cooled to room temperature and diluted with H₂O. The solution is extracted with AcOEt. The organic layer is washed 3 times with NaOH and brine, dried and evaporated in vacuo to give 4-[2-(2,4-dimethoxy-5-nitro-phenoxy)-ethyl]morpholine (1.7 g, 39%). The residue is reacted following Example A to afford 2,4-dimethoxy-5-(2-morpholin-4-yl-ethoxy)-phenylamine. ¹H-NMR CDCl3: 2.49-2.68 (m, 4H), 2.79 (t, 2H), 3.68-3.84 (m, 12H), 4.08 (t, 2H), 6.42 (s, 1H), 6.52 (s, 1H). Rf value: 0.40 (CH2Cl2:MeOH=10:1).

### Example I32

### Preparation of 2,4-Dimethoxy-5-morpholin-4-ylphenylamine

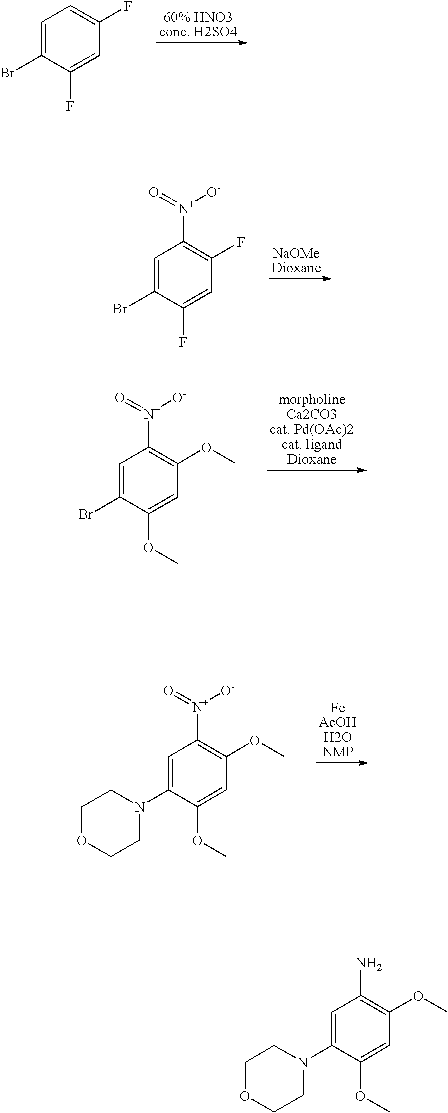

To a solution of 1-bromo-2,4-difluorobenzene (0.13 mol) in conc. H₂SO₄ (150 mL) is added dropwise 60% HNO₃ (30 mL) at 0° C. over 20 min. After stirring at 0-10° C. for 10 min, the reaction mixture is poured into ice-water (800 g) and extracted with ether. The separated organic layer is washed with sat. NaHCO₃ and brine, dried over MgSO₄ and evaporated in vacuo to give a crude 1-bromo-2,4-difluoro-5-nitrobenzene.

Rf: 0.57 (hexane:EtOAc=7:1). ¹H NMR (CDCl₃) δ 7.14 (dd, 1H), 8.39 (dd, 1H).

A mixture of 1-bromo-2,4-difluoro-5-nitrobenzene (0.010 mol) and NaOMe (0.050 mol) in dioxane (20 mL) is stirred and under reflux conditions for 24 h. After being cooled to room temperature, the reaction mixture is poured into water and extracted with EtOAc. The separated organic layer is washed with brine, dried over MgSO₄ and evaporated in vacuo to give 1-bromo-2,4-dimethoxy-5-nitrobenzene as brown solids.

Rf: 0.50 (hexane:EtOAc=1:1). ¹H NMR (CDCl₃) δ 3.999 (s, 3H), 4.001 (s, 3H), 6.52 (s, 1H), 8.25 (s, 1H).

A mixture of 1-bromo-2,4-dimethoxy-5-nitrobenzene (9.4 mmol), molpholine (14 mmol), Cs₂CO₃ (19 mmol), 2-(di-t-butylphosphino)biphenyl (3.7 mmol) and Pd(OAc)₂ (1.9 mmol) in dioxane (30 mL) is stirred under reflux conditions for 12 h. After being cooled to room temperature, the reaction mixture is poured into water and extracted with EtOAc. The separated organic layer is washed with brine, dried over MgSO₄ and evaporated in vacuo. The resulting residue is purified by silica gel column chromatography to give 4-(2,4-dimethoxy-5-nitrophenyl)morpholine.

Rf: 0.45 (EtOAc) ¹H NMR (CDCl₃) δ 3.00-3.05 (m, 4H), 3.86-3.90 (m, 4H), 3.976 (s, 3H), 3.978 (s, 3H), 6.53 (s, 1H), 7.62 (s, 1H).

To a suspension of iron (34 mmol), AcOH (1.5 mL), H₂O (3.0 mL) in N-methylpyrrolidone (6.0 mL) is added dropwise 4-(2,4-dimethoxy-5-nitrophenyl)morpholine (3.4 mmol) in N-methylpyrrolidone (8.0 mL) at 90° C. After stirring at 100° C. for 1.5 h, the reaction mixture is cooled to room temperature and quenched by sat NaHCO₃ aq. The reaction mixture is filtered through Celight and the residue is washed with EtOAc. The filtrate is extracted with EtOAc and separated organic layer is washed with H₂O and brine, dried over MgSO₄ and evaporated in vacuo. The resulting residue is purified by silica gel column chromatography to give 2,4-dimethoxy-5-morpholin-4-ylphenylamine.

Rf: 0.41 (EtOAc). ¹H NMR (CDCl₃) δ 2.95-3.00 (m, 4H), 3.51 (brs, 2H), 3.82 (s, 3H), 3.83 (s, 3H), 3.85-3.89 (m, 4H), 6.42 (s, 1H), 6.49 (s, 1H).

The following anilines are prepared according to the procedure described as Example 28 followed by hydrogenation as Example A.

| | | Identification: |
|---|---|---|
| Expl | | NMR (400 MHz) δ (ppm), ESI-MS, or Rf |
| No. | Structures | (solvent) |
| I33 | 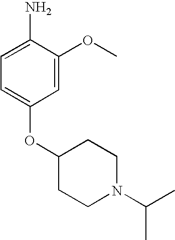 | DMSO: 1.23 (d, 6 H), 1.85-2.0 (m, 2 H), 2.2- 2.24 (m, 2 H), 2.68-2.8 (m, 2 H), 2.9-3.1 (m, 3 H), 3.79 (s, 3 H), 4.25-4.35 (m, 1 H), 6.34 (dd, 1 H), 6.44 (d, 1 H), 6.62 (dd, 1 H). Rf: 0.15 (CHCl3:MeOH = 10:1) |
| I34 | 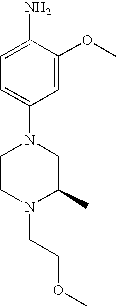 | DMSO: 1.13 (d, 6 H), 2.45-3.15 (m, 4 H), 2.8- 3.1 (m, 3 H), 3.15-3.65 (m, 9 H), 3.83 (s, 3 H), 6.4 (dd, 1 H), 6.5 (d, 1 H), 6.63 (d, 1 H). Rf: 0.57 (CHCl3:MeOH = 5:1) |
| I35 | 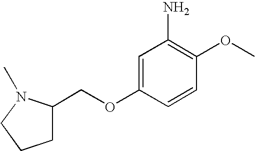 | CDCl3: 1.20-1.24 (m, 1 H), 1.67-1.89 (m, 4 H), 1.97-2.08 (m, 1 H), 2.25-2.33 (m, 1 H), 2.47 (s, 3 H), 3.11 (t, 1 H), 3.71-3.82 (m, 5 H), 3.91- 3.95 (m, 1 H), 6.25 (dd, 1 H), 6.35 (d, 1 H), 6.68 (d, 1 H). Rf value: 0.38 (CH2Cl2:MeOH = 5:1). |
| I36 | 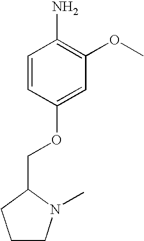 | CDCl3: 1.64-1.88 (m, 4 H), 1.97-2.07 (m, 1 H), 2.25-2.31 (m, 1 H), 2.48 (s, 3 H), 2.56-2.64 (m, 1 H), 3.10 (t, 1 H), 3.49 (brs, 2 H), 3.81 (s, 3 H), 3.91-395 (m, 1 H), 6.35 (dd, 1 H), 6.48 (d, 1 H), 6.63 (d, 1 H). Rf value: 0.41 (CH2Cl2:MeOH = 5:1). |

### Example A

### FAK Assay

All steps are performed in a 96-well black microtiter plate. Purified recombinant hexahistidine-tagged human FAK kinase domain is diluted with dilution buffer (50 mM HEPES, pH 7.5, 0.01% BSA, 0.05% Tween-20 in water) to a concentration of 94 ng/mL (2.5 nM). The reaction mixture is prepared by mixing 10 μL 5× kinase buffer (250 mM HEPES, pH 7.5, 50 μM Na₃VO₄, 5 mM DTT, 10 mM MgCl₂, 50 mM MnCl₂, 0.05% BSA, 0.25% Tween-20 in water), 20 μL water, 5 μL of 4 μM biotinylated peptide substrate (Biot-Y397) in aqueous solution, 5 μL of test compound in DMSO, and 5 μL of recombinant enzyme solution and incubated for 30 min at room temperature. The enzyme reaction is started by addition of 5 μL of 5 μM ATP in water and the mixture is incubated for 3 hours at 37° C. The reaction is terminated by addition of 200 μL of detection mixture (1 nM Eu-PT66 (Perkin Ekmer, No. AD0068), 2.5 μg/mL SA-(SL)APC (Perkin Elmer, No. CR130-100), 6.25 mM EDTA in dilution buffer), and the FRET signal from europium to allophycocyanin is measured by EnVision multilabel reader (Perkin Elmer) after 30 min of incubation at room temperature. The ratio of fluorescence intensity of 665 nm to 615 nm is used as a FRET signal for data analysis in order to cancel the colour quenching effect by a test compound. The results are shown as percent inhibition of enzyme activity. The level of the background signal is determined under the conditions without ATP, while DMSO is used as a control of 0% inhibition. IC₅₀ values are determined by non-linear curve fit analysis using the OriginPro 6.1 program (OriginLab).

The Biot-Y397 peptide (Biotin-SETDDYAEIID ammonium salt) is designed to have the same amino acid sequence as the region from S392 to D402 of human FAK (GenBank Accession Number L13616) and is prepared by standard methods.

Purified recombinant hexahistidine-tagged human FAK kinase domain is obtained in the following way: Full-length human FAK cDNA is isolated by PCR amplification from human placenta Marathon-Ready™ cDNA (Clontech, No. 7411-1) with the 5′ PCR primer (ATGGCAGCTGCTTACCTTGAC) and the 3′ PCR primer (TCAGTGTGGTCTCGTCTGCCC) and subcloned into a pGEM-T vector (Promega, No. A3600). After digestion with AccIII, the purified DNA fragment is treated with Klenow fragment. The cDNA fragment is digested with BamHI and cloned into pFastBacHTb plasmid (Invitrogen, 10584-027) previously cut with BamHI and Stu I. The resultant plasmid, hFAK KD (M384-G706)/pFastBacHTb, is sequenced to confirm its structure. The resulting DNA encodes a 364 amino acid protein containing a hexahistidine tag, a spacer region and a rTEV protease cleavage site at the N-terminal and the kinase domain of FAK (Met384-Gly706) from position 29 to 351.

Donor plasmid is transposed into the baculovirus genome, using MaxEfficacy DH10Bac E. coli cells (Invitrogen, No. 10361-012). Bacmid DNA is prepared by a simple alkaline lysis protocol described in the Bac-to-Bac® Baculovirus Expression system (Invitrogen, No. 10359-016). Sf9 insect cells are transfected based on the protocol provided by the vendor (CellFECTIN®, Invitrogen). The expression of FAK in each lysate is analysed by SDS-PAGE and Western blotting with anti-human FAK monoclonal antibody (Transduction Laboratories, No. F15020).

The virus clone that shows the highest expression is further amplified by infection to Sf9 cells. For large scale expression, amplified virus was infected to Expression in ExpresSF+® cells with 5 MOI for 72 hrs, these conditions gives high level of protein with little degradation. Cell lysates are loaded onto a column of HiTraP™ Chelating Sepharose HP (Amersham Biosciences, No. 17-0409-01) charged with nickel sulfate and equilibrated with 50 mM HEPES pH 7.5, 0.5 M NaCl and 10 mM imidazole. Captured protein is eluted with increasing amounts of imidazole in HEPES buffer/NaCl, and the buffer is exchanged to 50 mM HEPES pH 7.5, 10% glycerol and 1 mM DTT by dialysis.

### Example B

### Phosphorylation Levels of FAK

Phosphorylation levels of FAK at Tyr397 is quantified by the sandwich ELISA. Mouse mammary carcinoma 4T1 cells (1×10⁵) are plated in wells of 96-well culture plates and incubated with or without various concentrations of inhibitors for 1 h in Dulbecco's modified eagle medium containing 0.5% BSA. The medium is removed and cells are lysed in 200 μL 50 mM Tris-HCl, pH 7.4, containing 1% NP-40, 0.25% sodium deoxycholate, 150 mM NaCl, 1 mM EDTA, 1 mM PMSF, 1 mM Na₃VO₄, 1 mM NaF, 1 μg/mL aprotinin, 1 μg/mL leupeptin and 1 μg/mL pepstatin. After centrifugation, the supernatants are subjected to a sandwich ELISA to quantify the phosphorylated FAK and total FAK. Cell lysates are applied to 96-well flat-bottom ELISA plates which have been pre-coated with 100 μL/well of 4 μg/mL mouse monoclonal anti-FAK antibody (clone 77, Becton Dickinson Transduction Laboratories) in 50 mM Tris-HCl, pH 9.5, containing 150 mM NaCl for 18 h at 4° C. and blocked with 300 μL of BlockAce (Dainippon Pharmaceuticals Co.) diluted at 1:4 with H₂O at room temperature for 2 h. After washing with TBSN (20 mM Tris-HCl, pH 8.3, containing 300 mM NaCl, 0.1% SDS and 0.05% NP-40), total FAK is detected with 100 μL of 1 μg/ml anti-FAK polyclonal antibody (#65-6140, Upstate Biology Inc.), and phosphorylated FAK is detected with 100 μL of 0.25 μg/μL anti-phosphorylated FAK (Y397) antibody (Affinity BioReagents, #OPA1-03071) in BlockAce diluted at 1:10 with H₂O. After 1 h incubation at room temperature, plates are washed with TBSN and 100 μl of biotinylated anti-rabbit IgG (#65-6140, Zymed Laboratories Inc.) diluted at 1:2000 with BlockAce diluted at 1:10 with H₂O is incubated at room temperature for 1 h. After washing with TBSN, ABTS solution substrate kit (#00-2011, Zymed Laboratories Inc.) is used for color development. Absorbance at 405 nm is measured after 20 min incubation at room temperature. The concentration of compound causing 50% reduction of phosphorylation level of FAK is determined.

### Example C

### Anchorage-Independent Tumor Cell Growth Assay

Mouse mammary carcinoma 4T1 cells (5×10³) are plated in 96-well Ultra low Attachment plates (#3474, Corning Inc.) in 100 μL of Dulbecco's modified eagle medium containing 10% FBS. Cells are cultured for 2 h and inhibitors are added at various concentrations in a final concentration of 0.1% DMSO. After 48 h, cell growth is assayed with the cell counting kit-8 (Wako Pure Chemical), which uses a water soluble tetrazolium salt WST8. Twenty μL of the reagent is added into each well and cells are further cultured for 2 h. The optical density is measured at 450 nm. The concentration of compound causing 50% inhibition of growth is determined.

### Example D

### In Vitro T Cell Migration Assay

Inhibitory activities of FAK inhibitors on the mobility of immune cells are secured by the following in vitro study. That is, Jurkat-T human leukemic cell line are placed at 1×10⁵ cells in the upper chamber of Fluoroblok with 8 μm pores (Beckton Dickinson, UK), and are allowed to migrate by four hours cultivation at 37° C., in 95% air-5% CO2 depending on a concentration gradient of fetal bovine serum (10% FBS). Cell mobility is appraised through the number of cells migrated into lower chamber by labeling with calcein-AM (Molecular Probes, Netherlands) at 8 μg/ml in HBSS for 1 h. For evaluation of FAK inhibitors, both the upper and lower chambers are added with various concentrations of FAK inhibitors (0.03-10 μM). IC₅₀ values are calculated by the decrement of those fluorescent intensity compared to that in vehicle-treated group measured with Ascent (Ex: 485 nm, Em: 538 nm).

### Example E

### ALK Assay

The inhibition of ALK tyrosine kinase activity is measured using known methods, for example using the recombinant kinase domain of the ALK in analogy to the VEGF-R kinase assay described in J. Wood et al. Cancer Res. 60, 2178-2189 (2000).

The compounds of formula I potently inhibit the growth of human NPM-ALK overexpressing murine BaF3 cells. The expression of NPM-ALK is achieved by transfecting the BaF3 cell line with an expression vector PClneo™ (Promega Corp., Madison Wis., USA) coding for NPM-ALK and subsequent selection of G418 resistant cells. Non-transfected BaF3 cells depend on IL-3 for cell survival. In contrast NPM-ALK expressing BaF3 cells (named BaF3-NPM-ALK) can proliferate in the absence of IL-3 because they obtain proliferative signal through NPM-ALK kinase. Putative inhibitors of the NPM-ALK kinase therefore abolish the growth signal and result in antiproliferative activity. The antiproliferative activity of putative inhibitors of the NPM-ALK kinase can however be overcome by addition of IL-3 which provides growth signals through an NPM-ALK independent mechanism. [for an analogous cell system using FLT3 kinase see E Weisberg et al. Cancer Cell; 1, 433-443 (2002). The inhibitory activity of the compounds of formula I is determined, briefly, as follows: BaF3-NPM-ALK cells (15 000/microtitre plate well) are transferred to 96-well microtitre plates. The test compounds [dissolved in dimethyl sulfoxide (DMSO)] are added in a series of concentrations (dilution series) in such a manner that the final concentration of DMSO is not greater than 1% (v/v). After the addition, the plates are incubated for two days during which the control cultures without test compound are able to undergo two cell-division cycles. The growth of the BaF3-NPM-ALK cells is measured by means of Yopro™ staining (T Idziorek et al. J. Immunol. Methods; 185:249-58 [1995]): 25 μl of lysis buffer consisting of 20 mM sodium citrate, pH 4.0, 26.8 mM sodium chloride, 0.4% NP40, 20 mM EDTA and 20 mM was added to each well. Cell lysis was completed within 60 min at room temperature and total amount of Yopro bound to DNA was determined by measurement using the Cytofluor II 96-well reader (PerSeptive Biosystems) with the following settings: Excitation (nm) 485/20 and Emission (nm) 530/25.

IC₅₀ values are determined by a computer-aided system using the formula:

IC₅₀=[(ABSₜₑₛₜ−ABSₛₜₐᵣₜ)/(ABS_{control}−ABSₛₜₐᵣₜ)]×100.

The IC₅₀ value in those experiments is given as that concentration of the test compound in question that results in a cell count that is 50% lower than that obtained using the control without inhibitor. The compounds of formula I exhibit inhibitory activity with an IC₅₀ in the range from approximately 0.01 to 1 μM.

The antiproliferative action of the compounds of formula I can also be determined in the human KARPAS-299 lympoma cell line (described In W G Dirks et al. Int. J. Cancer 100, 49-56 (2002) using the same methodology described above for the BaF3-NPM-ALK cell line. The compounds of formula I exhibit inhibitory activity with an IC₅₀ in the range from approximately 0.01 to 1 μM.

### Example F

### Test for Activity Against IGF-I Induced IGF-IR Autophosphorylation Using the Cellular “Capture ELISA” Test

The assay is conducted as follows:

For the assay NIH-3T3 mouse fibroblasts transfected with human IGF-IR cDNA (complete human IGF-IR cDNA: GenBank Acc. No. NM_{—}000875), prepared as described in Kato et al., J. Biol. Chem. 268, 2655-61, 1993, are used. The cells which overexpress human IGF-IR are cultured in Dulbecco's minimal essential (DMEM) medium, containing 10% Fetal Calf Serum (FCS). For the assay 5,000 cells/well are plated on day 1 on 96-well plates (Costar #3595) in normal growth medium and incubated for 2 days at 37° C. in a standard CO₂ cell incubator. The density of the cells does not exceed 70-80% at day 3. On day 3 the medium is discarded and the cells are incubated for 24 h in minimal medium (DMEM, containing 0.5% FCS). Compounds of formula I [starting from 10 mM dimethyl sulfoxide (DMSO) stock solutions] are added to produce final concentrations of 0.01, 0.03, 0.1, 0.3, 1, 3 and 10 μM to determine the IC₅₀ value. The cells are incubated for 90 min in the presence of a compound of formula I. Thereafter the cells are stimulated with 50 μl IGF-I (final concentration of IGF-I in the well=10 ng/ml; IGF-I is obtained from Sigma; Product Code: I 3769) and incubated for 10 min at 37° C. The medium is discarded and the cells are washed twice with PBS/O (=Phosphate-Buffered Saline without CaCl₂) and lysed for 15 min on ice with 50 μl/well RIPA-buffer [50 mM Tris.HCl, pH=7.2, 120 mM NaCl, 1 mM EDTA, 6 mM EGTA, 1% NP-40, 20 mM NaF, 1 mM benzamidine, 15 mM sodium pyrophosphate, 1 mM Phenyl methyl sulphonyl fluoride (PMSF) and 0.5 mM Na₃VO₄] and shaken for 10 min using a 96-well plate shaker (=cellular extracts).

Packard HTRF-96 black plates are coated with 50 μl IGF-IR monoclonal Antibody (mAB) (Santa Cruz; Cat. No.: SC-462) in a concentration of 5 μg/ml at 4° C. overnight. The plates are washed twice with 0.05% (v/v) Tween-20 in Phosphate-Buffered Saline (PBS) and once with nanopure H₂O. Blocking is done for 2 h at room temperature (RT) with 3% Bovine Serum Albumin (BSA) in TBS-T buffer (20 mM Tris.HCl, pH=7.6, 137 mM NaCl, 0.05% Tween-20). After blocking, the plates are washed once with nanopure H₂O.

Cellular extracts (40 μl/well) are pipetted onto the precoated Packard plates, together with 40 μl of the anti-phosphotyrosine mouse mAB PY-20 conjugated with Alkaline Phosphatase (AP) (1:1000 diluted in RIPA buffer; the antibody is obtained from Transduction Labs; Cat. No.: P11120).

After incubating the extracts and the secondary antibody for 2 h at 4° C., the extracts are discarded, the plates are washed twice with 0.05% (v/v) Tween-20 in PBS and once with nanopure water.

90 μl/well AP substrate (CDP-Star; obtained from Tropix; Cat. No.: MS100RY) are then added and the plates are incubated for 45 min at RT in the dark, followed by measuring AP activity in a Packard Top Count Microplate Scintillation Counter. The IC₅₀ values for the compounds of formula I are calculated via linear regression analysis using the GraphPad Instat program (GraphPad Software, USA). IC₅₀ values in the range of 5 nM to 1 μM, especially in the range of 5 nM to 300 nM are found.

### Example G In Vivo Activity in the Nude Mouse Xenograft Model

female or male BALB/c nude mice (5-8 weeks old, Charles River Japan, Inc., Yokohama, Japan) are kept under sterile conditions with water and feed ad libitum. Tumours are induced by subcutaneous injection of tumour cells (human epithelial cell line MIA PaCa-2; European Collection of Cell Cultures (ECACC), Salisbury, Wiltshire, UK, Catalogue Number 85062806; cell line from a 65 year old Caucasian male; undifferentiated human pancreatic carcinoma cell line) into left or right flank of mice under Forene® anaesthesia (Abbott Japan Co., Ltd., Tokyo, Japan). Treatment with the test compound is started when the mean tumor volumes reached approximately 100 mm³. Tumour growth is measured two times per week and 1 day after the last treatment by determining the length of two perpendicular axis. The tumour volumes are calculated in accordance with published methods (see Evans et al., Brit. J. Cancer 45, 466-8, 1982). The anti-tumour efficacy is determined as the mean increase in tumour volume of the treated animals divided by the mean increase in tumour volume of the untreated animals (controls) and, after multiplication by 100, is expressed as delta T/C [%]. Tumour regression is reported as the mean changes of tumor volume of the treated animals divided by the mean tumor volume at start of treatment and, after multiplication by 100, is expressed as regression [%]. The test compound is orally administered daily with or without drug holidays.

As an alternative to cell line MIA PaCa-2, another cell line may also be used in the same manner, for example:
the 4T1 breast carcinoma cell line (ATCC Number CRL-2539; see also Cancer. 88 (12 Supple), 2979-2988, 2000) with female BALB/c mice (injection into mammary fat pad).

### Example H

### Tablets

Tablets comprising 50 mg of active ingredient, for example one of the compounds of formula I described in Examples 1 to 131, and having the following composition are prepared in customary manner:

### Composition:

| | | |
|---|---|---|
| active ingredient | 50 | mg |
| wheat starch | 150 | mg |
| lactose | 125 | mg |
| colloidal silicic acid | 12.5 | mg |
| talc | 22.5 | mg |
| magnesium stearate | 2.5 | mg |
| Total: | 362.5 | mg |

Preparation: The active ingredient is mixed with a portion of the wheat starch, with the lactose and the colloidal silicic acid and the mixture is forced through a sieve. A further portion of the wheat starch is made into a paste, on a water bath, with five times the amount of water and the powder mixture is kneaded with the paste until a slightly plastic mass is obtained.

The plastic mass is pressed through a sieve of about 3 mm mesh size and dried, and the resulting dry granules are again forced through a sieve. Then the remainder of the wheat starch, the talc and the magnesium stearate are mixed in and the mixture is compressed to form tablets weighing 145 mg and having a breaking notch.

### Example I

### Soft Capsules

5000 soft gelatin capsules comprising each 50 mg of active ingredient, for example one of the compounds of formula I described in Examples 1 to 131, are prepared in customary manner:

### Composition:

| | | |
|---|---|---|
| active ingredient | 250 | g |
| Lauroglykol | 2 | litres |

Preparation: The pulverized active ingredient is suspended in Lauroglykol® (propylene glycol laurate, Gattefossé S. A., Saint Priest, France) and ground in a wet pulverizer to a particle size of approx. 1 to 3 μm. 0.419 g portions of the mixture are then dispensed into soft gelatin capsules using a capsule-filling machine.

1. A compound of formula I 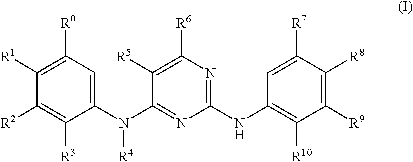
wherein
R₀ is hydrogen
R₁ is hydrogen or a 5 or 6 member heterocycl comprising 1 or 2 N atoms substituted by C₁-C₇alkyl, hydroxy, dialkylamino, or by a 6 member heterocycl comprising 1 N atom;
R₂ is hydrogen
R₃ is sulfamoyl substituted once or twice by C₁-C₇alkyl; carbamoyl substituted once or twice by C₁-C₇alkyl; 5 or 6 member heterocycl comprising 1, 2, 3 or 4 N atoms; SO2N(R₁₂)R₁₃ wherein R₁₂ is hydrogen or loweralkyl and R₁₃ is hydrogen, C₁-C₇alkyl, C₁-C₇alkoxy-C₁-C₇alkyl, di-C₁-C₇alkylamino-C₁-C₇alkyl, hydroxy-C₁-C₇alkyl or R₁₂ and R₁₃ together with the N to which they are attached form a heterocycl comprising 2 N atoms which is unsubstituted or substituted C₁-C₇alkyl;
R₂ and R₃ together with the N to which they are attached form a heterocycl comprising 2 hetero atoms independently selected from N or S which is unsubstituted or substituted once or twice by a substituent independently selected from loweralkyl and oxo;
R₄ is hydrogen
R₅ is halogen
R₆ is hydrogen
R₇ is hydrogen; C₁-C₇alkoxy; carbamoyl unsubstituted or substituted by loweralkyl; 5 or 6 member heterocycl comprising 1, 2 or 3 N or O atoms unsubstituted or substituted by di-C₁-C₇alkyl-amino, C₁-C₇alkyl, hydroxy, 5 or 6 member heterocycl comprising 1, 2 or 3 N or O atoms unsubstituted or substituted by C₁-C₇alkyl; 5 or 6 member heterocycloxy comprising 1, 2 or 3 N or O ring atoms unsubstituted or substituted by C₁-C₇alkyl; heterocycl-C₁-C₇alkyloxy wherein heterocycl is a 5 or 6 member heterocycl comprising 1, 2 or 3 N or O ring atoms unsubstituted or substituted by hydroxy or C₁-C₇alkyl;
R₈ is hydrogen; halogen; C₁-C₇alkoxy; carbamoyl unsubstituted or substituted by C₁-C₇alkyl; heterocycl-C₁-C₇alkyloxy wherein heterocycl is a 5 or 6 member heterocycl comprising 1, 2 or 3 N or O ring atoms unsubstituted or substituted by C₁-C₇alkyl hydroxy; 5 or 6 member heterocycl comprising 1, 2 or 3 N or O atoms unsubstituted or substituted once or twice by a substituent independently selected from hydroxy C₁-C₇alkoxy-C₁-C₇alkyl, C₁-C₇ aminocarbonyl and C₁-C₇alkylamino; 5 or 6 member heterocycloxy comprising 1 or 2 N ring atoms unsubstituted or substituted 1 to 5 times by C₁-C₇alkyl or di-C₁-C₇alkylamino; 10 member bi-cyclic-heterocycle comprising 1 to 3 heteroatoms selected from N or O;
R₇ and R₈ together with the atoms to which they are attached form a 6 member heterocycl comprising 1, 2 or 3 N or O atoms unsubstituted or substituted once or twice by C₁-C₇alkyl or oxo;
R₉ is hydrogen, 5 or 6 member heterocycl comprising 1, 2 or 3 N or O atoms unsubstituted or substituted by di-C₁-C₇alkyl-amino;
R₁₀ is hydrogen or C₁-C₇alkoxy;
or salts thereof.

2. A compound of formula I according to claim 1, wherein
R₀ is hydrogen
R₁ is hydrogen or
R₂ is hydrogen
R₃ is SO₂N(R₁₂)R₁₃ wherein R₁₂ is hydrogen or C₁-C₇alkyl and R₁₃ is hydrogen, C₁-C₇alkyl, C₁-C₇alkoxy-C₁-C₇alkyl, di-C₁-C₇alkylamino-C₁-C₇alkyl, hydroxy-C₁-C₇alkyl;
R₄ is hydrogen
R₅ is Br or Cl
R₆ is hydrogen
R₇ is hydrogen; C₁-C₇alkoxy; carbamoyl unsubstituted or substituted by loweralkyl; 5 or 6 member heterocycl comprising 1, 2 or 3 N or O atoms unsubstituted or substituted by di-C₁-C₇alkyl-amino, C₁-C₇alkyl, hydroxy 5 or 6 member heterocycl comprising 1, 2 or 3 N or O atoms unsubstituted or substituted by C₁-C₇alkyl; 5 or 6 member heterocycloxy comprising 1, 2 or 3 N or O ring atoms unsubstituted or substituted by C₁-C₇alkyl; heterocycl-C₁-C₇alkyloxy wherein heterocycl is a 5 or 6 member heterocycl comprising 1, 2 or 3 N or O ring atoms unsubstituted or substituted by hydroxy or C₁-C₇alkyl;
R₈ is hydrogen; halogen; C1-C7alkoxy; carbamoyl unsubstituted or substituted by C₁-C₇alkyl; heterocycl-C₁-C₇alkyloxy wherein heterocycl is a 5 or 6 member heterocycl comprising 1, 2 or 3 N or O ring atoms unsubstituted or substituted by C₁-C₇alkyl hydroxy; 5 or 6 member heterocycl comprising 1, 2 or 3 N or O atoms unsubstituted or substituted once or twice by a substituent independently selected from hydroxy C₁-C₇alkoxy-C₁-C₇alkyl, C₁-C₇alkyl, aminocarbonyl and C₁-C₇alkylamino; 5 or 6 member heterocycloxy comprising 1 or 2 N ring atoms unsubstituted or substituted 1 to 5 times by C₁-C₇alkyl or di-C₁-C₇alkylamino; 10 member bi-cyclic-heterocycle comprising 1 to 3 heteroatoms selected from N or O;
R₉ is hydrogen;
R₁₀ is C₁-C₇alkoxy;
or salts thereof

3. A compound of formula I according to claim 1 wherein
R₀ is hydrogen
R₁ is hydrogen or
R₂ is hydrogen
R₃ is SO₂N(R₁₂)R₁₃ wherein R₁₂ is hydrogen or C₁-C₇alkyl and R₁₃ is hydrogen, C₁-C₇alkyl, C₁-C₇alkoxy-C₁-C₇alkyl, di-C₁-C₇alkylamino-C₁-C₇alkyl, hydroxy-C₁-C₇alkyl;
R₄ is hydrogen
R₅ is Br or Cl
R₆ is hydrogen
R₇ is hydrogen;
R₈ is hydrogen; halogen; C1-C7alkoxy; carbamoyl unsubstituted or substituted by C₁-C₇alkyl; heterocycl-C₁-C₇alkyloxy wherein heterocycl is a 5 or 6 member heterocycl comprising 1, 2 or 3 N or O ring atoms unsubstituted or substituted by C₁-C₇alkyl hydroxy; 5 or 6 member heterocycl comprising 1, 2 or 3 N or O atoms unsubstituted or substituted once or twice by a substituent independently selected from hydroxy C₁-C₇alkoxy-C₁-C₇alkyl, C₁-C₇alkyl, aminocarbonyl and C₁-C₇alkylamino; 5 or 6 member heterocycloxy comprising 1 or 2 N ring atoms unsubstituted or substituted 1 to 5 times by C₁-C₇alkyl or di-C₁-C₇alkylamino; 10 member bi-cyclic-heterocycle comprising 1 to 3 heteroatoms selected from N or O;
R₉ is hydrogen;
R₁₀ is C₁-C₇alkoxy;
or salts thereof

4. The compound of formula I according to claim 1 selected from;
2-{5-Bromo-2-[5-(3-dimethylamino-pyrrolidin-1-yl)-2-methoxy-phenylamino]-pyrimidin-4-ylamino}-N-methyl-benzenesulfonamide,
2-{5-Bromo-2-[2-methoxy-5-(2-morpholin-4-yl-ethoxy)-phenylamino]-pyrimidin-4-ylamino}-N-isopropyl-benzenesulfonamide,
7-[2-(4-[1,4′]Bipiperidinyl-1′-yl-2-methoxy-phenylamino)-5-chloro-pyrimidin-4-ylamino]-2-methyl-2,3-dihydro-isoindol-1-one,
2-[5-Chloro-2-(2-methoxy-4-morpholin-4-yl-phenylamino)-pyrimidin-4-ylamino]-5-(4-hydroxy-piperidin-1-yl)-N-methyl-benzamide,
5-[1,4′]Bipiperidinyl-1′-yl-2-[5-chloro-2-(2-methoxy-5-morpholin-4-yl-phenylamino)-pyrimidin-4-ylamino]-N-methyl-benzamide,
2-[2-(4-[1,4′]Bipiperidinyl-1′-yl-2-methoxy-phenylamino)-5-chloro-pyrimidin-4-ylamino]-N-isobutyl-benzenesulfonamide,
2-{5-Chloro-2-[2-methoxy-4-(4-methyl-piperazin-1-yl)-phenylamino]-pyrimidin-4-ylamino}-N-isobutyl-benzenesulfonamide,
2-[5-Chloro-2-(2-methoxy-5-morpholin-4-yl-phenylamino)-pyrimidin-4-ylamino]-5-(4-hydroxy-piperidin-1-yl)-N-methyl-benzamide,
2-[2-(5-[1,4′]Bipiperidinyl-1′-yl-2-methoxy-phenylamino)-5-bromo-pyrimidin-4-ylamino]-N-isopropyl-benzenesulfonamide,
1-{4-[5-Chloro-4-(2-isobutylsulfamoyl-phenylamino)-pyrimidin-2-ylamino]-3-methoxy-phenyl}-piperidine-4-carboxylic acid amide,
4-[5-Chloro-4-(2-isobutylsulfamoyl-phenylamino)-pyrimidin-2-ylamino]-3-methoxy-N-methyl-benzamide,
2-{5-Chloro-2-[4-(4-hydroxy-piperidin-1-yl)-2-methoxy-phenylamino]-pyrimidin-4-ylamino}-N-isobutyl-benzenesulfonamide,
3-[5-Chloro-4-(2-isobutylsulfamoyl-phenylamino)-pyrimidin-2-ylamino]-4-methoxy-N-methyl-benzamide,
5-Chloro-N²-(2-methoxy-4-morpholin-4-yl-phenyl)-N⁴-[2-(2H-tetrazol-5-yl)-phenyl]-pyrimidine-2,4-diamine,
2-{5-Chloro-2-[4-((S)-3-dimethylamino-pyrrolidin-1-yl)-2-methoxy-phenylamino]-pyrimidin-4-ylamino}-N-isobutyl-benzenesulfonamide,
2-{5-Chloro-2-[2-methoxy-4-(1-methyl-piperidin-4-yloxy)-phenylamino]-pyrimidin-4-ylamino}-N-isobutyl-benzenesulfonamide,
7-{5-Chloro-2-[4-((S)-3-dimethylamino-pyrrolidin-1-yl)-2-methoxy-phenylamino]-pyrimidin-4-ylamino}-2-methyl-2,3-dihydro-isoindol-1-one,
2-{5-Chloro-2-[4-((S)-3-dimethylamino-pyrrolidin-1-yl)-2-methoxy-phenylamino]-pyrimidin-4-ylamino}-N-methyl-5-(4-methyl-piperazin-1-yl)-benzamide,
1-{4-[5-Chloro-4-(2-methylcarbamoyl-phenylamino)-pyrimidin-2-ylamino]-3-methoxy-phenyl}-3-methyl-piperidine-3-carboxylic acid amide,
1-{4-[5-Chloro-4-(2-methyl-3-oxo-2,3-dihydro-1H-isoindol-4-ylamino)-pyrimidin-2-ylamino]-3-methoxy-phenyl}-3-methyl-piperidine-3-carboxylic acid amide,
1-{4-[5-Chloro-4-(2-isobutylsulfamoyl-phenylamino)-pyrimidin-2-ylamino]-3-methoxy-phenyl}-3-methyl-piperidine-3-carboxylic acid amide,
2-{5-Chloro-2-[5-(3-dimethylamino-pyrrolidin-1-yl)-2-methoxy-phenylamino]-pyrimidin-4-ylamino}-N-methyl-5-(4-methyl-piperazin-1-yl)-benzamide,
7-{5-Chloro-2-[2-methoxy-4-(1-methyl-piperidin-4-yloxy)-phenylamino]-pyrimidin-4-ylamino}-2-methyl-2,3-dihydro-isoindol-1-one,
2-[5-Bromo-2-(2,5-dimethoxy-4-morpholin-4-yl-phenylamino)-pyrimidin-4-ylamino]-N-methyl-benzenesulfonamide,
2-{5-Bromo-2-[5-(4-hydroxy-piperidin-1-yl)-2-methoxy-phenylamino]-pyrimidin-4-ylamino}-N-methyl-benzenesulfonamide,
2-[5-Chloro-2-(2-methoxy-5-morpholin-4-yl-phenylamino)-pyrimidin-4-ylamino]-N-isobutyl-benzenesulfonamide,
2-{5-Chloro-2-[2-methoxy-5-(4-methyl-piperazin-1-yl)-phenylamino]-pyrimidin-4-ylamino}-N-isobutyl-benzenesulfonamide,
2-[2-(5-[1,4′]Bipiperidinyl-1′-yl-2-methoxy-phenylamino)-5-chloro-pyrimidin-4-ylamino]-N-isobutyl-benzenesulfonamide,
2-{5-Chloro-2-[5-((S)-3-dimethylamino-pyrrolidin-1-yl)-2-methoxy-phenylamino]-pyrimidin-4-ylamino}-N-isobutyl-benzenesulfonamide,
1-{4-[5-Chloro-4-(2-methyl-3-oxo-2,3-dihydro-1H-isoindol-4-ylamino)-pyrimidin-2-ylamino]-3-methoxy-phenyl}-piperidine-4-carboxylic acid amide,
2-[5-Chloro-2-(2-methoxy-4-morpholin-4-yl-phenylamino)-pyrimidin-4-ylamino]-5-((S)-3-dimethylamino-pyrrolidin-1-yl)-N-methyl-benzamide,
7-{5-Chloro-2-[4-(4-isopropyl-piperazin-1-yl)-2-methoxy-phenylamino]-pyrimidin-4-ylamino}-2-methyl-2,3-dihydro-isoindol-1-one,
2-[5-Chloro-2-(2-methoxy-4-morpholin-4-yl-phenylamino)-pyrimidin-4-ylamino]-N-(2,2-dimethyl-propyl)-benzenesulfonamide,
2-{5-Chloro-2-[2-methoxy-4-(4-methyl-piperazin-1-yl)-phenylamino]-pyrimidin-4-ylamino}-N-(2,2-dimethyl-propyl)-benzenesulfonamide,
3-[5-Chloro-4-(2-isobutylsulfamoyl-phenylamino)-pyrimidin-2-ylamino]-4-methoxy-benzamide,
2-[5-Bromo-2-(2,4-dimethoxy-5-morpholin-4-yl-phenylamino)-pyrimidin-4-ylamino]-N-methyl-benzenesulfonamide,
2-{5-Bromo-2-[5-(1-isopropyl-piperidin-4-yloxy)-2-methoxy-phenylamino]-pyrimidin-4-ylamino}-N-methyl-benzenesulfonamide,
7-(5-Chloro-2-{2-methoxy-4-[2-(4-methyl-piperazin-1-yl)-ethoxy]-phenylamino}-pyrimidin-4-ylamino)-2-methyl-2,3-dihydro-isoindol-1-one,
2-(5-Chloro-2-{2-methoxy-4-[4-(4-methyl-piperazin-1-yl)-piperidin-1-yl]-phenylamino}-pyrimidin-4-ylamino)-N-isobutyl-benzenesulfonamide,
(S)-1-{4-[5-Chloro-4-(2-methyl-3-oxo-2,3-dihydro-1H-isoindol-4-ylamino)-pyrimidin-2-ylamino]-3-methoxy-phenyl}-3-methyl-piperidine-3-carboxylic acid amide,
(S)-1-{4-[5-Chloro-4-(2-methylcarbamoyl-phenylamino)-pyrimidin-2-ylamino]-3-methoxy-phenyl}-3-methyl-piperidine-3-carboxylic acid amide,
7-[5-Chloro-2-(2,4-dimethoxy-phenylamino)-pyrimidin-4-ylamino]-2-methyl-2,3-dihydro-isoindol-1-one,
2-(5-Bromo-2-{2-methoxy-5-[4-(4-methyl-piperazin-1-yl)-piperidin-1-yl]-phenylamino}-pyrimidin-4-ylamino)-N-methyl-benzenesulfonamide,
2-{5-Bromo-2-[5-(4-hydroxy-piperidin-1-yl)-2-methoxy-phenylamino]-pyrimidin-4-ylamino}-N-isopropyl-benzenesulfonamide,
2-{5-Bromo-2-[2-methoxy-5-(1-methyl-piperidin-4-yloxy)-phenylamino]-pyrimidin-4-ylamino}-N-isopropyl-benzenesulfonamide,
2-{5-Bromo-2-[5-(1-isopropyl-piperidin-4-yloxy)-2-methoxy-phenylamino]-pyrimidin-4-ylamino}-N-isopropyl-benzenesulfonamide,
7-{5-Chloro-2-[2-methoxy-4-(2-morpholin-4-yl-ethoxy)-phenylamino]-pyrimidin-4-ylamino}-2-methyl-2,3-dihydro-isoindol-1-one,
7-{5-Chloro-2-[2-methoxy-5-(2-morpholin-4-yl-ethoxy)-phenylamino]-pyrimidin-4-ylamino}-2-methyl-2,3-dihydro-isoindol-1-one,
7-{5-Chloro-2-[4-(1-isopropyl-piperidin-4-yloxy)-2-methoxy-phenylamino]-pyrimidin-4-ylamino}-2-methyl-2,3-dihydro-isoindol-1-one,
2-{5-Bromo-2-[5-(3-dimethylamino-pyrrolidin-1-yl)-2-methoxy-phenylamino]-pyrimidin-4-ylamino}-N-isopropyl-benzenesulfonamide,
2-{5-Bromo-2-[2-methoxy-5-(4-methyl-piperazin-1-yl)-phenylamino]-pyrimidin-4-ylamino}-N-isopropyl-benzenesulfonamide,
2-(5-Bromo-2-{2-methoxy-5-[4-(4-methyl-piperazin-1-yl)-piperidin-1-yl]-phenylamino}-pyrimidin-4-ylamino)-N-isopropyl-benzenesulfonamide,
7-{5-Chloro-2-[2-methoxy-4-(1,2,2,6,6-pentamethyl-piperidin-4-yloxy)-phenylamino]-pyrimidin-4-ylamino}-2-methyl-2,3-dihydro-isoindol-1-one,
1-{4-[5-Chloro-4-(2-methyl-3-oxo-2,3-dihydro-1H-isoindol-4-ylamino)-pyrimidin-2-ylamino]-3-methoxy-phenyl}-piperidine-3-carboxylic acid amide,
2-{5-Chloro-2-[2-methoxy-4-(1,2,2,6,6-pentamethyl-piperidin-4-yloxy)-phenylamino]-pyrimidin-4-ylamino}-N-isobutyl-benzenesulfonamide,
(R)-1-{4-[5-Chloro-4-(2-methylcarbamoyl-phenylamino)-pyrimidin-2-ylamino]-3-methoxy-phenyl}-3-methyl-piperidine-3-carboxylic acid amide,
(R)-1-{4-[5-Chloro-4-(2-methyl-3-oxo-2,3-dihydro-1H-isoindol-4-ylamino)-pyrimidin-2-ylamino]-3-methoxy-phenyl}-3-methyl-piperidine-3-carboxylic acid amide,
2-{5-Chloro-2-[2-methoxy-4-((R{1-methyl-pyrrolidin-2-ylmethoxy)-phenylamino]-pyrimidin-4-ylamino}-N-isobutyl-benzenesulfonamide,
2-{5-Chloro-2-[2-methoxy-4-((S)-1-methyl-pyrrolidin-2-ylmethoxy)-phenylamino]-pyrimidin-4-ylamino}-N-isobutyl-benzenesulfonamide,
2-{5-Bromo-2-[2-methoxy-5-(2-piperidin-1-yl-ethoxy)-phenylamino]-pyrimidin-4-ylamino}-N-methyl-benzenesulfonamide,
2-(5-Bromo-2-{5-[2-(4-hydroxy-piperidin-1-yl)-ethoxy]-2-methoxy-phenylamino}-pyrimidin-4-ylamino)-N-methyl-benzenesulfonamide,
5-Chloro-N⁴-(1,1-dioxo-1λ⁶-thiochroman-8-yl)-N²-(2-methoxy-4-morpholin-4-yl-phenyl)-pyrimidine-2,4-diamine,
2-[5-Chloro-2-(2-methoxy-4-morpholin-4-yl-phenylamino)-pyrimidin-4-ylamino]-N-(2-hydroxy-ethyl)-benzenesulfonamide,
2-[5-Chloro-2-(2-methoxy-4-morpholin-4-yl-phenylamino)-pyrimidin-4-ylamino]-N-(2-methoxy-ethyl)-benzenesulfonamide,
7-{5-Chloro-2-[2-methoxy-4-(2-piperidin-1-yl-ethoxy)-phenylamino]-pyrimidin-4-ylamino}-2-methyl-2,3-dihydro-isoindol-1-one,
2-[5-Chloro-2-(2-methoxy-4-morpholin-4-yl-phenylamino)-pyrimidin-4-ylamino]-N-((R)-2-hydroxy-propyl)-benzenesulfonamide,
2-[5-Chloro-2-(2-methoxy-4-morpholin-4-yl-phenylamino)-pyrimidin-4-ylamino]-N-(3-hydroxy-propyl)-benzenesulfonamide,
2-[5-Chloro-2-(2-methoxy-4-morpholin-4-yl-phenylamino)-pyrimidin-4-ylamino]-N-((S)-2-hydroxy-propyl)-benzenesulfonamide,
2-{5-Bromo-2-[2-methoxy-5-(4-morpholin-4-yl-piperidin-1-yl)-phenylamino]-pyrimidin-4-ylamino}-N-isopropyl-benzenesulfonamide,
7-(5-Chloro-2-{2-methoxy-4-[(S)-4-(2-methoxy-ethyl)-3-methyl-piperazin-1-yl]-phenylamino}-pyrimidin-4-ylamino)-2-methyl-2,3-dihydro-isoindol-1-one,
7-(5-Chloro-2-{2-methoxy-4-[(R)-4-(2-methoxy-ethyl)-3-methyl-piperazin-1-yl]-phenylamino}-pyrimidin-4-ylamino)-2-methyl-2,3-dihydro-isoindol-1-one,
5-Chloro-N²-[4-((S)-3-dimethylamino-pyrrolidin-1-yl)-2-methoxy-phenyl]-N⁴-(1,1-dioxo-1λ⁶-thiochroman-8-yl)-pyrimidine-2,4-diamine,
5-Chloro-N⁴-(1,1-dioxo-1λ⁶-thiochroman-8-yl)-N²-{2-methoxy-4-[4-(4-methyl-piperazin-1-yl)-piperidin-1-yl]-phenyl}-pyrimidine-2,4-diamine,
2-{5-Bromo-2-[2-methoxy-5-(4-morpholin-4-yl-piperidin-1-yl)-phenylamino]-pyrimidin-4-ylamino}-N-methyl-benzenesulfonamide,
2-[5-Bromo-2-(4-fluoro-2-methoxy-5-morpholin-4-yl-phenylamino)-pyrimidin-4-ylamino]-N-methyl-benzenesulfonamide,
4-[5-Chloro-4-(1,1-dioxo-1λ⁶-thiochroman-8-ylamino)-pyrimidin-2-ylamino]-3-methoxy-N-methyl-benzamide,
2-{5-Bromo-2-[2-methoxy-5-((S)-1-methyl-pyrrolidin-2-ylmethoxy)-phenylamino]-pyrimidin-4-ylamino}-N-methyl-benzenesulfonamide,
2-{5-Bromo-2-[2-methoxy-5-((R)-1-methyl-pyrrolidin-2-ylmethoxy)-phenylamino]-pyrimidin-4-ylamino}-N-methyl-benzenesulfonamide,
2-{5-Bromo-2-[2,4-dimethoxy-5-(2-morpholin-4-yl-ethoxy)-phenylamino]-pyrimidin-4-ylamino}-N-methyl-benzenesulfonamide,
2-[5-Chloro-2-(2-methoxy-4-morpholin-4-yl-phenylamino)-pyrimidin-4-ylamino]-N-isopropyl-N-methyl-benzenesulfonamide,
2-[5-Chloro-2-(2-methoxy-4-morpholin-4-yl-phenylamino)-pyrimidin-4-ylamino]-N-methyl-N-propyl-benzenesulfonamide,
7-(5-Chloro-2-{4-[2-(4-isopropyl-piperazin-1-yl)-ethoxy]-2-methoxy-phenylamino}-pyrimidin-4-ylamino)-2-methyl-2,3-dihydro-isoindol-1-one,
2-{5-Bromo-2-[2-methoxy-5-(2-morpholin-4-yl-ethoxy)-phenylamino]-pyrimidin-4-ylamino}-N,N-dimethyl-benzenesulfonamide,
2-[5-Bromo-2-(2,4-dimethoxy-5-morpholin-4-yl-phenylamino)-pyrimidin-4-ylamino]-N-isopropyl-benzenesulfonamide,
2-[5-Chloro-2-(2-methoxy-4-morpholin-4-yl-phenylamino)-pyrimidin-4-ylamino]-N-(2-dimethylamino-ethyl)-benzenesulfonamide,
5-Chloro-N²-(2-methoxy-4-morpholin-4-yl-phenyl)-N⁴-[2-(4-methyl-piperazine-1-sulfonyl)-phenyl]-pyrimidine-2,4-diamine,
2-[5-Chloro-2-(2-methoxy-4-morpholin-4-yl-phenylamino)-pyrimidin-4-ylamino]-N-(2-ethoxy-ethyl)-benzenesulfonamide,
2-[5-Bromo-2-(7-methoxy-4-methyl-3-oxo-3,4-dihydro-2H-benzo[1,4]oxazin-6-ylamino)-pyrimidin-4-ylamino]-N-methyl-benzenesulfonamide,
2-[5-Bromo-2-(2-methoxy-5-morpholin-yl-phenylamino)-pyrimidin-4-ylamino]-N,N-dimethyl-benzenesulfonamide,
2-[5-Bromo-2-(2-methoxy-5-morpholin-4-yl-phenylamino)-pyrimidin-4-ylamino]-N,N-dimethyl-benzenesulfonamide,
2-{5-Chloro-2-[2-methoxy-4-(4-methyl-piperazin-1-yl)-phenylamino]-pyrimidin-4-ylamino}-N-methyl-N-propyl-benzenesulfonamide,
2-[5-Bromo-2-(2-methoxy-5-piperidin-1-yl-phenylamino)-pyrimidin-4-ylamino]-N-methyl-benzenesulfonamide,
2-[5-Bromo-2-(2-methoxy-5-piperidin-1-yl-phenylamino)-pyrimidin-4-ylamino]-N-isopropyl benzenesulfonamide,
7-{5-Chloro-2-[4-((R)-3-dimethylamino-pyrrolidin-1-yl)-2-methoxy-phenylamino]-pyrimidin-4-ylamino}-2-methyl-2,3-dihydro-isoindol-1-one,
5-Chloro-N²-(2-methoxy-4-morpholin-4-yl-phenyl)-N⁴-[2-(piperazine-1-sulfonyl)-phenyl]-pyrimidine-2,4-diamine,
2-[5-Chloro-2-(2-methoxy-4-morpholin-4-yl-phenylamino)-pyrimidin-4-ylamino]-N-isobutyl-N-methyl-benzenesulfonamide,
2-[5-Chloro-2-(2-methoxy-4-morpholin-4-yl-phenylamino)-pyrimidin-4-ylamino]-N-ethyl-N-methyl-benzenesulfonamide,
2-{5-Chloro-2-[2-methoxy-4-(4-methyl-piperazin-1-yl)-phenylamino]-pyrimidin-4-ylamino}-N-isobutyl-N-methyl-benzenesulfonamide,
2-{5-Chloro-2-[2-methoxy-4-(4-methyl-piperazin-1-yl)-phenylamino]-pyrimidin-4-ylamino}-N-ethyl-N-methyl-benzenesulfonamide,
7-(5-Chloro-2-{4-[2-(4-hydroxy-piperidin-1-yl)-ethoxy]-2-methoxy-phenylamino}-pyrimidin-4-ylamino)-2-methyl-2,3-dihydro-isoindol-1-one,
2-(5-Bromo-2-{2-methoxy-5-[4-(4-methyl-piperazin-1-yl)-piperidin-1-yl]-phenylamino}-pyrimidin-4-ylamino)-N,N-dimethyl-benzenesulfonamide,
8-{5-Chloro-2-[2-methoxy-4-(4-methyl-piperazin-1-yl)-phenylamino]-pyrimidin-4-ylamino}-2-methyl-3,4-dihydro-2H-isoquinolin-1-one,
8-[5-Chloro-2-(2-methoxy-4-morpholin-4-yl-phenylamino)-pyrimidin-4-ylamino]-2-methyl-3,4-dihydro-2H-isoquinolin-1-one,
8-(5-Chloro-2-{2-methoxy-4-[4-(4-methyl-piperazin-1-yl)-piperidin-1-yl]-phenylamino}-pyrimidin-4-ylamino)-2-methyl-3,4-dihydro-2H-isoquinolin-1-one,
8-[2-(4-[1,4′]Bipiperidinyl-1′-yl-2-methoxy-phenylamino)-5-chloro-pyrimidin-4-ylamino]-2-methyl-3,4-dihydro-2H-isoquinolin-1-one,
8-{5-Chloro-2-[4-(4-hydroxy-piperidin-1-yl)-2-methoxy-phenylamino]-pyrimidin-4-ylamino}-2-methyl-3,4-dihydro-2H-isoquinolin-1-one,
8-{5-Chloro-2-[4-(4-isopropyl-piperazin-1-yl)-2-methoxy-phenylamino]-pyrimidin-4-ylamino}-2-methyl-3,4-dihydro-2H-isoquinolin-1-one,
7-(5-Chloro-2-{2-methoxy-4-[3-(4-methyl-piperazin-1-yl)-propoxy]-phenylamino}-pyrimidin-4-ylamino)-2-methyl-2,3-dihydro-isoindol-1-one,
8-{5-Chloro-2-[4-((S)-3-dimethylamino-pyrrolidin-1-yl)-2-methoxy-phenylamino]-pyrimidin-4-ylamino}-2-methyl-3,4-dihydro-2H-isoquinolin-1-one,
8-{5-Chloro-2-[4-((R)-3-dimethylamino-pyrrolidin-1-yl)-2-methoxy-phenylamino]-pyrimidin-4-ylamino}-2-methyl-3,4-dihydro-2H-isoquinolin-1-one,
8-[5-Chloro-2-((S)-4-hexahydro-pyrazino[2,1-c][1,4]oxazin-8-yl-2-methoxy-phenylamino)-pyrimidin-4-ylamino]-2-methyl-3,4-dihydro-2H-isoquinolin-1-one,
8-[5-Chloro-2-((R)-4-hexahydro-pyrazino[2,1-c][1,4]oxazin-8-yl-2-methoxy-phenylamino)-pyrimidin-4-ylamino]-2-methyl-3,4-dihydro-2H-isoquinolin-1-one,
8-[5-Chloro-2-(2-methoxy-4-morpholin-4-yl-phenylamino)-pyrimidin-4-ylamino]-2-ethyl-3,4-dihydro-2H-isoquinolin-1-one,
8-(5-Chloro-2-{2-methoxy-4-[4-(4-methyl-piperazin-1-yl)-piperidin-1-yl]-phenylamino}-pyrimidin-4-ylamino)-2-ethyl-3,4-dihydro-2H-isoquinolin-1-one,
2-[5-Chloro-2-(2-methoxy-5-morpholin-4-yl-phenylamino)-pyrimidin-4-ylamino]-N-methyl-5-(4-methyl-piperazin-1-yl)-benzamide,
5-[1,4′]Bipiperidinyl-1′-yl-2-[5-chloro-2-(2-methoxy-4-morpholin-4-yl-phenylamino)-pyrimidin-4-ylamino]-N-methyl-benzamide,
2-[5-Bromo-2-(2-methoxy-5-morpholin-4-yl-phenylamino)-pyrimidin-4-ylamino]-N-isopropyl-benzenesulfonamide,
5-Chloro-N²-{4-[4-(4-ethyl-piperazin-1-yl)-piperidin-1-yl]-2-methoxy-phenyl}-N⁴-[2-(propane-2-sulfonyl)-phenyl]-pyrimidine-2,4-diamine,
2-{5-Chloro-2-[4-((S)-3-ethylamino-pyrrolidin-1-yl)-2-methoxy-phenylamino]-pyrimidin-4-ylamino}-N-isopropyl-benzenesulfonamide,
2-{5-Chloro-2-[4-((R)-3-ethylamino-pyrrolidin-1-yl)-2-methoxy-phenylamino]-pyrimidin-4-ylamino}-N-isopropyl-benzenesulfonamide,
2-{5-Chloro-2-[2-methoxy-4-((S)-3-methylamino-pyrrolidin-1-yl)-phenylamino]-pyrimidin-4-ylamino}-N-isopropyl-benzenesulfonamide,
2-{5-Chloro-2-[2-methoxy-4-((R)-3-methylamino-pyrrolidin-1-yl)-phenylamino]-pyrimidin-4-ylamino}-N-isopropyl-benzenesulfonamide,
2-{5-Chloro-2-[4-((R)-3-dimethylamino-pyrrolidin-1-yl)-2-methoxy-phenylamino]-pyrimidin-4-ylamino}-N-isopropyl-benzenesulfonamide,
2-{5-Chloro-2-[4-((S)-3-dimethylamino-pyrrolidin-1-yl)-2-methoxy-phenylamino]-pyrimidin-4-ylamino}-N-isopropyl-benzenesulfonamide,
2-{5-Chloro-2-[2-ethoxy-4-(4-methyl-piperazin-1-yl)-phenylamino]-pyrimidin-4-ylamino}-N-isopropyl-benzenesulfonamide,
2-{5-Chloro-2-[2-isopropoxy-4-(4-methyl-piperazin-1-yl)-phenylamino]-pyrimidin-4-ylamino}-N-isopropyl-benzenesulfonamide,
2-{5-Chloro-2-[2-cyclopropylmethoxy-4-(4-methyl-piperazin-1-yl)-phenylamino]-pyrimidin-4-ylamino}-N-isopropyl-benzenesulfonamide
and salts thereof.

5. A process for the production of a compound of formula I according to claim 1, comprising reacting a compound of formula II 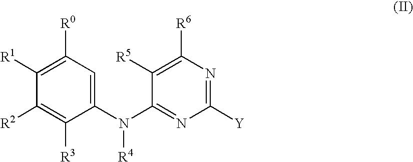
wherein R⁰, R¹, R², R³, R⁴, R⁵, and R⁶ are as defined in claim 1, and Y is a leaving group, with a compound of formula III 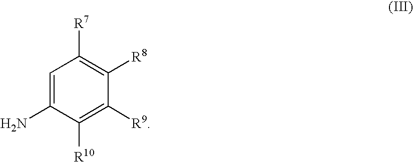
wherein R⁷, R⁸, R⁹ and R¹⁰ are as defined in claim 1;
and, if desired, converting a compound of formula I, wherein the substituents have the meaning as defined in claim 1, into another compound of formula I as defined in claim 1;
and recovering the resulting compound of formula I in free from or as a salt, and, when required, converting the compound of formula I obtained in free form into the desired salt, or an obtained salt into the free form.

6. A pharmaceutical composition comprising a compound of formula I according to claim 1, as active ingredient together with one or more pharmaceutically acceptable diluents or carriers.

7. (canceled)

8. A combination comprising a therapeutically effective amount a compound of formula I according to claim 1 and one or more further drug substances, said further drug substance being useful in the treatment of neoplastic diseases or immune system disorders.

9. A method for the treatment of neoplastic diseases and immune system disorders in a subject in need thereof which comprises administering an effective amount of a compound of formula I, according to claim 1, or a pharmaceutical composition comprising same.

10. A method for the treatment or prevention of a disease which responds to inhibition of the FAK, ALK and/or IGF-1 Receptor comprising administering a compound of formula I, according to claim 1, or a pharmaceutically acceptable salt thereof.

11. The method according to claim 10, wherein the disease to be treated is selected from proliferative disease.

12. The method according to claim 11, wherein the proliferative disease to be treated is selected from a tumor of, breast, renal, prostate, colorectal, thyroid, ovarian, pancreas, neuronal, lung, uterine and gastrointestinal tumours as well as osteosarcomas and melanomas.
